(19) United States
(12) Patent Application Publication
(10) Pub. No.: US 2024/0002902 A1
(43) Pub. Date: Jan. 4, 2024

(54) ANALYSIS OF ANTIGEN AND ANTIGEN RECEPTOR INTERACTIONS

(71) Applicant: 10x Genomics, Inc., Pleasanton, CA (US)

(72) Inventors: Søren Nyboe JAKOBSEN, Charlottenlund (DK); Malte KÜHNEMUND, Stockholm (SE)

(73) Assignee: 10x Genomics, Inc., Pleasanton, CA (US)

(21) Appl. No.: 18/313,251

(22) Filed: May 5, 2023

Related U.S. Application Data
(60) Provisional application No. 63/339,378, filed on May 6, 2022.

(51) Int. Cl. C12Q 1/6804; C12Q 1/6874

(52) U.S. Cl. CPC C12Q 1/6804; C12Q 1/6874

(57) ABSTRACT

The present disclosure relates in some aspects to methods of contacting a biological sample (e.g., fresh or frozen tissue sample) with peptide-loaded detection complexes. In some aspects, the peptide-loaded detection complexes comprise antigen presenting molecule monomers or multimers that bind to antigens, reporter oligonucleotides that correspond to the antigen/antigen presenting molecule combination. In some aspects, the peptide-loaded detection complexes are tetramers. In some aspects, the tetramers bound to T cell receptors of T cells may be detected in situ. In some aspects, the tetramers bound to TCRs may be detected on an in situ platform and/or using an array comprising spatially barcoded capture agents. In some embodiments, the method comprises contacting a plurality of peptide-loaded detection complexes to clonal populations of T cells comprising TCRs of different antigen specificities.

### CROSS-REFERENCE TO RELATED APPLICATION

This application claims priority to U.S. Provisional Patent Application No. 63/339,378, filed May 6, 2022, entitled “ANALYSIS OF ANTIGEN AND ANTIGEN RECEPTOR INTERACTIONS,” which is herein incorporated by reference in its entirety for all purposes.

### FIELD

The present disclosure relates in some aspects to detection of interactions between antigens and binding partners, such as antigen receptors and/or antigen responsive cells in a biological sample.

### BACKGROUND

Immune cells express antigen receptors which mediate diverse immune responses initiated by the binding of the receptors to various antigens. For instance, T cell-mediated recognition of peptide-major histocompatibility complex (pMHC) complexes is crucial for the control of intracellular pathogens and cancer, as well as for stimulation and maintenance of efficient cytotoxic responses. However, spatial organization of an organism's TCR repertoire in various anatomic compartments is not well understood. Tools are needed to provide insights into the spatial distribution of the antigen binding specificities of various antigen receptors in tissues. Provided herein are methods, compositions, and kits that address such and other needs.

### BRIEF SUMMARY

Insights into the mechanisms of antigen and antigen receptor interactions, including the spatial organization of such interactions in tissues, are crucial to understanding disease development and establishing new treatment strategies. MHC multimers have been used for detection of antigen-responsive T cells in liquid samples using flow cytometry techniques. However, challenges persist in developing fast and reliable platforms for screening of antigen and antigen receptor (e.g., pMHC and TCR) interactions with high sensitivity and at high throughput.

The present disclosure relates in some aspects to analyzing the spatial distribution of various interactions between antigens and binding partners in a biological sample, such as the antigen binding specificities of T cell receptors (TCRs) or antigen responsive T cells. In some embodiments, disclosed herein are detection complexes comprising antigen presenting molecules (APMs) (e.g., MHCs) for high throughput antigen/epitope identification and receptor specificity determination. In some embodiments, the detection complexes are antigen-loaded prior to contacting a biological sample to be analyzed. In some embodiments, the detection complexes are used in an in situ platform where signals (e.g., optical signals) associated with the detection complexes are detected in cell or tissue samples. In some embodiments, the detection complexes are used in a spatial array platform where spatially barcoded oligonucleotides are sequenced to reveal the spatial information of the antigen receptors in cell or tissue samples.

In some aspects, provided herein is a method for analyzing a biological sample, comprising: a) contacting the biological sample with an antigen and a detection complex simultaneously or in any order, wherein: the detection complex can comprise an antigen presenting molecule (APM) monomer or multimer that binds to the antigen to form an antigen-loaded detection complex, wherein the antigen-loaded detection complex is conjugated to a reporter oligonucleotide, the reporter oligonucleotide corresponds to the antigen or a portion thereof or the antigen/APM combination in the antigen-loaded detection complex, and the antigen-loaded detection complex binds to a receptor on and/or in a cell at a location in the biological sample; b) contacting the biological sample with a probe or probe set that hybridizes to the reporter oligonucleotide; and c) detecting a signal associated with the probe or probe set or a product thereof at the location in the biological sample.

In some embodiments, the detection complex can be bound to the antigen to form the antigen-loaded detection complex prior to the contacting of the biological sample with the antigen and the detection complex. In some embodiments, through the contacting of the biological sample with the antigen and the detection complex, the detection complex becomes loaded by binding to the antigen to form the antigen-loaded detection complex.

In any of the embodiments herein, the antigen presenting molecule (APM) multimer can comprise 2, 3, 4, 5, 6, 7, 8, 9, 10, or more antigen presenting molecules (e.g., MHC molecules). In any of the embodiments herein, the antigen-loaded detection complex can comprise 2, 3, 4, 5, 6, 7, 8, 9, 10, or more molecules of the antigen. In any of the embodiments herein, the antigen and the APM can form an antigen-APM monomer, e.g., an antigen peptide and an MHC molecule can form a peptide-MHC monomer. In any of the embodiments herein, the antigen-loaded detection complex can comprise one or more dimers, tetramers, pentamers, octamers, streptamers, or dodecamers of an antigen-APM monomer. In any of the embodiments herein, the antigen-loaded detection complex can comprise a dimer, tetramer, pentamer, octamer, streptamer, or dodecamer of a peptide-MHC (pMHC) complex.

In any of the embodiments herein, the antigen can comprise a peptide, a protein, a nucleic acid, a lipid, a carbohydrate, a metabolite, or a small molecule, or a combination thereof. In any of the embodiments herein, the antigen can comprise a fatty acid, a phospholipid, or a glycolipid, or a combination thereof.

In any of the embodiments herein, the APM can comprises an MHC molecule, an MHC-like molecule, or a combination thereof. In any of the embodiments herein, the APM can be a Class I MHC molecule or a Class II MHC molecule. In any of the embodiments herein, the APM can be a Class I MHC molecule comprising an α subunit and a β2 microglobulin subunit. In any of the embodiments herein, the APM can be a Class II MHC molecule comprising an α subunit and a β subunit.

In any of the embodiments herein, the antigen-loaded detection complex can comprise a scaffold covalently or non-covalently conjugated to the APM monomer or multimer. In any of the embodiments herein, the conjugation may be via one or more linkers. In any of the embodiments herein, the scaffold can comprise a polysaccharide, a streptavidin, an avidin, or mutant, variant, or analog thereof. In any of the embodiments herein, the polysaccharide can be a glucan. In any of the embodiments herein, the polysaccharide can comprise dextran. In any of the embodiments herein, the streptavidin, avidin, or mutant, variant, or analog thereof may be a tetramer comprising four subunits. In the tetramer, any two or more of the subunits can be covalently or non-covalently linked. In any of the embodiments herein, the scaffold can comprise a homotetramer or heterotetramer of the streptavidin, avidin, or mutant, variant, or analog thereof.

In any of the embodiments herein, the APM can be an MHC molecule and the antigen-loaded detection complex can be a peptide-loaded detection complex. In any of the embodiments herein, the peptide-loaded detection complex can comprise four MHC molecules each linked to a biotin or mutant, variant, or analog thereof that binds to one of the four subunits of a tetramer of a streptavidin, avidin, or mutant, variant, or analog thereof. In any of the embodiments herein, each MHC molecule can be bound to one molecule of an antigen peptide. In any of the embodiments herein, the antigen peptide may be between 5 and 40 amino acid residues in length, inclusive. In any of the embodiments herein, the antigen peptide may be between about 7 and about 15 amino acid residues in length, inclusive. In any of the embodiments herein, the antigen peptide may be between about 10 and about 25 amino acid residues in length, inclusive.

In any of the embodiments herein, the cell can be an immune cell. In any of the embodiments herein, the cell can be a T cell, B cell, or an NKT cell. In any of the embodiments herein, the cell can be a cell comprising a T cell receptor (TCR). In any of the embodiments herein, the receptor on and/or in the cell can be a pre-TCR or a mature TCR. In any of the embodiments herein, the receptor on and/or in the cell can be an αβ TCR or a γδ TCR. In any of the embodiments herein, the antigen-loaded detection complex can comprise four APM molecules (e.g., MHC molecules), where one or more or all four of the APM molecules can be bound to an antigen, such as an antigen peptide. In any of the embodiments herein, the antigen-loaded detection complex can comprise one or more pMHC complexes, each capable of binding to a TCR on and/or in the cell in the biological sample.

In any of the embodiments herein, the reporter oligonucleotide can be covalently or non-covalently conjugated to the antigen. In any of the embodiments herein, the conjugation can be via one or more linkers. In any of the embodiments herein, the reporter oligonucleotide can be covalently or non-covalently conjugated to the APM monomer or multimer (e.g., an MHC monomer or multimer). In any of the embodiments herein, the conjugation can be via one or more linkers. In any of the embodiments herein, the reporter oligonucleotide can be covalently or non-covalently conjugated to a scaffold of the antigen-loaded detection complex. In any of the embodiments herein, the conjugation can be via one or more linkers. In some embodiments, the scaffold comprises a streptavidin, a avidin, or mutant, variant, or analog thereof. In some embodiments, the scaffold comprises a polysaccharide, e.g., a glucan such as dextran.

In any of the embodiments herein, the reporter oligonucleotide can be used to identify the antigen or portion thereof. In any of the embodiments herein, the reporter oligonucleotide can be used to identify the antigen/APM combination, such as an antigen peptide/MHC combination, in the antigen-loaded detection complex. In any of the embodiments herein, the reporter oligonucleotide and/or a product thereof can be detected in situ in the biological sample, e.g., using a probe or probe set. In any of the embodiments herein, the reporter oligonucleotide and/or a product thereof can be captured on a substrate and detected. In any of the embodiments herein, the reporter oligonucleotide may but does not need to comprise a sequence encoding the antigen (e.g., a peptide sequence in the antigen).

In any of the embodiments herein, the reporter oligonucleotide can comprise a barcode region for identifying the antigen or portion thereof. In any of the embodiments herein, the barcode region can be a contiguous region or a non-contiguous region. In any of the embodiments herein, the barcode region can be a split region. In any of the embodiments herein, the barcode region can comprise one or more barcode sequences. In any of the embodiments herein, the barcode sequence can be a contiguous barcode sequence or a non-contiguous barcode sequence. In any of the embodiments herein, the barcode sequence can be a split barcode sequence. In any of the embodiments herein, the reporter oligonucleotide can comprise a capture region, a unique molecular identifier (UMI) region, a primer binding region, and/or an adapter region. In any of the embodiments herein, the capture region or a complement thereof can be captured by a capture agent on a substrate.

In any of the embodiments herein, the biological sample may not be fixed and/or crosslinked prior to the contacting of the biological sample with the antigen and/or the detection complex. In any of the embodiments herein, the biological sample can be a freshly isolated cell or tissue sample. In any of the embodiments herein, the biological sample can be a sectioned (e.g., cryosectioned) tissue sample. In any of the embodiments herein, the biological sample can be a fresh frozen tissue sample, such as a sectioned (e.g., cryosectioned) freshly isolated tissue sample.

In any of the embodiments herein, the biological sample can be contacted with a first matrix-forming material prior to the contacting of the biological sample with the antigen and/or the detection complex. In any of the embodiments herein, the method can comprise forming a first polymerized matrix from the first matrix-forming material, thereby embedding the biological sample in the first polymerized matrix. In some embodiments, the first matrix-forming material does not crosslink biological molecules in the biological sample. In some embodiments, the first matrix-forming material does not react with biological molecules in the biological sample. In some embodiments, protein molecules in the biological sample are not crosslinked by the first matrix-forming material or to the first polymerized matrix. In some embodiments, molecules of the receptor (e.g., TCRs) in the biological sample remain capable of binding to the APM and the antigen. In some embodiments, TCRs in the biological sample remain capable of binding to pMHC complexes of peptide-loaded detection complexes. In any of the embodiments herein, the first polymerized matrix can be a hydrogel. In any of the embodiments herein, the first matrix-forming material can comprise polyethylene glycol (PEG) or a derivative or analog thereof comprising a functional group for crosslinking. In any of the embodiments herein, the functional group can be a click functional group. In any of the embodiments herein, the method can further comprise washing the biological sample embedded in the first polymerized matrix.

In any of the embodiments herein, prior to and/or during the contacting of the biological sample with the antigen and the detection complex, the receptor (e.g., TCRs) in the biological sample can specifically bind to the one or more antigen-APM monomers (e.g., pMHC complexes) in the antigen-loaded detection complex, and sample processing prior to and/or during the contacting of the biological sample with the antigen and/or the detection complex does not disrupt or reduce the ability of the receptors to specifically bind to the antigen-APM complex in the antigen-loaded detection complex.

In any of the embodiments herein, the method can further comprise fixing and/or crosslinking the biological sample after the contacting of the biological sample with the antigen and the detection complex and prior to the contacting of the biological sample with the probe or probe set. In some embodiments, a sample can be fixed and/or crosslinked after pMHC/TCR interaction. In any of the embodiments herein, the antigen-loaded detection complex (e.g., a pMHC tetramer) and the receptor (e.g., a TCR) bound thereto in the biological sample can be fixed and/or crosslinked after the contacting of the biological sample with the antigen and/or the detection complex. In some embodiments, the biological sample can be contacted with a second matrix-forming material after the contacting of the biological sample with the antigen and the detection complex. In some embodiments, the biological sample can be contacted with the second matrix-forming material after fixing the biological sample. In some embodiments, the method can comprise forming a second polymerized matrix from the second matrix-forming material, thereby embedding the biological sample in the second polymerized matrix. In some embodiments, the second matrix-forming material can crosslink the antigen-loaded detection complex (e.g., a pMHC tetramer) and the receptor (e.g., a TCR) bound thereto to each other, to other molecules in the biological sample, and/or to the second polymerized matrix.

In any of the embodiments herein, the antigen-loaded detection complex may bind to one, two, three, four, or more molecules of the receptor. In any of the embodiments herein, the antigen-loaded detection complex may not be fluorescent. In any of the embodiments herein, the antigen-loaded detection complex may not be fluorescently labeled. In some embodiments, the antigen-loaded detection complex is not covalently linked to a fluorescent label.

In any of the embodiments herein, the method can further comprise removing antigen-loaded detection complexes that are unbound or nonspecifically bound to the receptor from the biological sample, prior to the contacting of the biological sample with the probe or probe set. In any of the embodiments herein, the removing can be performed prior to and/or after fixing and/or crosslinking the biological sample. In any of the embodiments herein, the removing can comprise washing the biological sample, e.g., under a condition that disrupts nonspecific binding of antigen-loaded detection complexes to the biological sample while preserving specific binding between the antigen-APM complex(es) in an antigen-loaded detection complex and a receptor in the biological sample.

In any of the embodiments herein, the probe or one or more probe molecules in the probe set can be fluorescent or fluorescently labeled. In any of the embodiments herein, the probe or probe set can comprise a covalently linked fluorophore. In any of the embodiments herein, the probe or probe set may not be fluorescent or fluorescently labeled. In any of the embodiments herein, the probe or probe set can directly or indirectly bind to a fluorescent or fluorescently labeled probe. In any of the embodiments herein, the probe or one or more probe molecules in the probe set can each hybridize to one or more fluorescent or fluorescently labeled probes. In any of the embodiments herein, the probe or probe set may be linear, circular, or circularizable.

In any of the embodiments herein, upon hybridization to the reporter oligonucleotide, the probe or probe set can be ligatable using the reporter oligonucleotide and/or a splint as template. In any of the embodiments herein, the probe or probe set can be ligatable with or without gap filling prior to ligation. In some embodiments, the splint may but does not need to be configured to hybridize to the reporter oligonucleotide. In some embodiments, the method can comprise ligating the probe or probe set using the reporter oligonucleotide and/or the splint as template, thereby circularizing the probe or probe set. In some embodiments, the probe or probe set hybridized to the reporter oligonucleotide can be ligated using one or more ligases and/or using chemical ligation.

In any of the embodiments herein, the product of the probe or probe set can be a rolling circle amplification (RCA) product. In any of the embodiments herein, the RCA can comprise a linear RCA, a branched RCA, a dendritic RCA, or any combination thereof. In any of the embodiments herein, the probe or probe set can comprise a region that can hybridize to a complementary barcode region in the reporter oligonucleotide. In some embodiments, the reporter oligonucleotide comprises a barcode region complementary to a sequence of the probe. In any of the embodiments herein, the probe or probe set can comprise a probe barcode region that is not configured to hybridize to the reporter oligonucleotide. In some embodiments, the reporter oligonucleotide does not comprise a complement of the probe barcode region. In some embodiments, the RCA product can comprise multiple copies of a sequence in the barcode region of the reporter oligonucleotide and/or multiple copies of the complement of the probe barcode region. In some embodiments, the detecting of signal(s) associated with the probe or probe set or a product thereof can comprise contacting the RCA product with detectable probes configured to hybridize to the complements or sequences thereof in sequential hybridization cycles. In some embodiments, the detectable probes can be fluorescent or fluorescently labeled. In some embodiments, the detectable probes each can hybridize to one or more fluorescent or fluorescently labeled probes.

In any of the embodiments herein, the reporter oligonucleotide and/or the probe or probe set can comprise a region which is an initiator for hybridization chain reaction (HCR) or which can hybridize to an initiator for HCR. In any of the embodiments herein, the reporter oligonucleotide and/or the probe or probe set can comprise a region which is an initiator for linear oligonucleotide hybridization chain reaction (LO-HCR) or which can hybridize to an initiator for LO-HCR. In any of the embodiments herein, the reporter oligonucleotide and/or the probe or probe set can comprise a region which is a primer for primer exchange reaction (PER) or which can hybridize to a primer for PER. In any of the embodiments herein, the reporter oligonucleotide and/or the probe or probe set can comprise a region which is a pre-amplifier for branched DNA (bDNA) or which can hybridize to a pre-amplifier for bDNA.

In any of the embodiments herein, the detecting of signal(s) associated with the probe or probe set or a product thereof can comprise imaging the biological sample to detect the signal. In any of the embodiments herein, the imaging can comprise fluorescent microscopy. In any of the embodiments herein, the detecting of signal(s) associated with the probe or probe set or a product thereof can comprise sequential hybridization, sequencing by hybridization, sequencing by ligation, sequencing by synthesis, sequencing by binding, or a combination thereof. In any of the embodiments herein, the detecting of signal(s) associated with the probe or probe set or a product thereof may but does not need to comprise contacting the biological sample with an antibody that binds to the antigen-loaded detection complex. In any of the embodiments herein, the method does not comprise contacting the biological sample with an antibody that binds to the antigen-loaded detection complex. In some embodiments, the method does not comprise detecting the antibody.

In any of the embodiments herein, cells or nuclei in the biological sample do not need to be partitioned and are preferably not partitioned in individual partitions, such as emulsion droplets and/or microwells. In any of the embodiments herein, the biological sample can be immobilized on a substrate prior to the contacting with the antigen, detection complex, or antigen-loaded detection complex. In any of the embodiments herein, the substrate can be a planar substrate, such as a slide or slip, e.g., a glass or plastic slide or a glass or plastic coverslip.

In some aspects, provided herein is a method for analyzing a biological sample, comprising: a) contacting the biological sample with a plurality of peptide-loaded detection complexes, wherein: each peptide-loaded detection complex can comprise a major histocompatibility complex (MHC) multimer and molecules of an antigen peptide bound to the MHC molecules of the multimer, wherein the MHC multimer can comprise a scaffold conjugated to a reporter oligonucleotide comprising a barcode region corresponding to the antigen peptide or a portion thereof or the antigen peptide/MHC combination in the peptide-loaded detection complex; b) allowing the plurality of peptide-loaded detection complexes to bind to T cell receptors (TCRs) of T cells at multiple locations in the biological sample; c) fixing and/or crosslinking the biological sample; d) contacting the fixed and/or crosslinked biological sample with a plurality of probes or probe sets that each hybridizes to the barcode region or a portion thereof; and e) detecting a signal associated with the probe or probe set or a product thereof at a particular location in the biological sample, thereby detecting a particular reporter oligonucleotide and the corresponding antigen peptide or antigen peptide/MHC combination at the particular location.

In any of the embodiments herein, the corresponding antigen peptide or antigen peptide/MHC combination can indicate an antigen binding specificity of one or more TCRs at the particular location. In some embodiments, the antigen binding specificity of one or more TCRs at the particular location in the biological sample is detected by detecting the antigen peptide or antigen peptide/MHC combination at the particular location.

In any of the embodiments herein, the method can further comprise detecting a TCR transcript encoding a component of the TCR specific to the antigen peptide or antigen peptide/MHC combination at the particular location. In any of the embodiments herein, the TCR transcript can comprise a TCRα V-J join, a TCRβ V-D-J join, a TCRγ V-J join, or a TCRδ V-D-J join. In any of the embodiments herein, a signal associated with the TCR transcript can be detected at the particular location in the biological sample. In some embodiments, a spatially labeled polynucleotide can be sequenced, wherein the spatially labeled polynucleotide can comprise (i) a sequence of the TCR transcript or complement thereof and (ii) a sequence of a spatial barcode or complement thereof, and the spatial barcode can correspond to a location on a substrate corresponding to the particular location in the biological sample.

In any of the embodiments herein, the plurality of peptide-loaded detection complexes can comprise antigen peptides or antigen peptide/MHC combinations for binding to TCRs of different antigen specificities. In any of the embodiments herein, the biological sample can comprise clonal populations of T cells expressing TCRs of different antigen specificities.

In any of the embodiments herein, cells or nuclei in the biological sample do not need to be partitioned and are preferably not partitioned in individual partitions, such as emulsion droplets and/or microwells. In any of the embodiments herein, the biological sample can be immobilized on a substrate prior to the contacting with the antigen, detection complex, or antigen-loaded detection complex. In any of the embodiments herein, the substrate can be a planar substrate, such as a slide or slip, e.g., a glass or plastic slide or a glass or plastic coverslip.

Also provided herein is a method for analyzing a biological sample, comprising: a) contacting the biological sample with an antigen and a detection complex simultaneously or in any order, wherein: the detection complex comprises an antigen-presenting molecule (APM) monomer or multimer that binds to the antigen to form an antigen-loaded detection complex, wherein the antigen-loaded detection complex is conjugated to a reporter oligonucleotide, the reporter oligonucleotide corresponds to the antigen or a portion thereof or the antigen/APM combination in the antigen-loaded detection complex and comprises a capture region, and the antigen-loaded detection complex binds to a receptor on and/or in a cell at a location in the biological sample; b) capturing the reporter oligonucleotide or a portion thereof by a capture agent, where in the capture agent is at a location on a substrate and comprises: i) a capture domain that binds to the capture region, and ii) a spatial barcode corresponding to the location of the capture agent on the substrate and the corresponding location in the biological sample; c) generating a spatially labeled polynucleotide comprising (i) a sequence of the reporter oligonucleotide or complement thereof and (ii) a sequence of the spatial barcode or complement thereof; and d) determining a sequence of the spatially labeled polynucleotide to detect the spatial barcode and the reporter oligonucleotide, thereby detecting the corresponding antigen or antigen/APM combination at the location in the biological sample.

In any of the embodiments herein, the APM can be an MHC molecule, the antigen can be an antigen peptide, and the receptor can be a TCR. In any of the embodiments herein, the same or a different capture agent can capture a sequence of a TCR transcript or a portion thereof. In any of the embodiments herein, the TCR transcript can comprise a TCRα V-J join, a TCRβ V-D-J join, a TCRγ V-J join, and/or a TCRδ V-D-J join. In any of the embodiments herein, the TCR transcript can encode a component of the TCR specific to the antigen peptide or antigen peptide/MHC combination at the location in the biological sample. In any of the embodiments herein, the spatially labeled polynucleotide can be a first spatially labeled polynucleotide, the method can further comprise generating a second spatially labeled polynucleotide comprising (i) a sequence of the TCR transcript or complement thereof and (ii) a sequence of the spatial barcode or complement thereof.

In any of the embodiments herein, the biological sample can be on the substrate, wherein the reporter oligonucleotide, the TCR transcript, or portion thereof is not captured prior to the capturing of the reporter oligonucleotide or portion thereof by the capture agent. In any of the embodiments herein, the substrate can be a planar substrate, such as a slide or slip, e.g., a glass or plastic slide or a glass or plastic coverslip. In any of the embodiments herein, the substrate can be a first substrate, the biological sample can be on a second substrate, and the biological sample can be sandwiched between the first and second substrates to allow capture of the reporter oligonucleotide, the TCR transcript, or portion thereof. In any of the embodiments herein, each of the first and second substrates can be a planar substrate, such as a slide or slip, e.g., a glass or plastic slide or a glass or plastic coverslip.

In any of the embodiments herein, the biological sample can be processed to release the reporter oligonucleotide, the TCR transcript, or portion thereof prior to the capturing of the reporter oligonucleotide or portion thereof by the capture agent. In any of the embodiments herein, the processing can comprise permeabilizing and/or lysing the biological sample. In any of the embodiments herein, the spatially labeled polynucleotide or a portion thereof can be removed from the substrate for determining the sequence of the spatially labeled polynucleotide. In any of the embodiments herein, the sequence of the spatially labeled polynucleotide can be determined using nucleic acid sequencing.

In any of the embodiments herein, the reporter oligonucleotide and/or the capture agent can comprise a unique molecular identifier (UMI) region, a primer binding region, and/or an adapter region.

In any of the embodiments herein, the biological sample may not be fixed and/or crosslinked prior to the contacting of the biological sample with the antigen and/or the detection complex. In any of the embodiments herein, the biological sample can be a freshly isolated cell or tissue sample. In any of the embodiments herein, the biological sample can be a sectioned (e.g., cryosectioned) tissue sample. In any of the embodiments herein, the method may comprise but does not require imaging the biological sample using fluorescent microscopy to detect the sequence of the reporter oligonucleotide or complement thereof and/or the sequence of the spatial barcode or complement thereof.

In some embodiments, disclosed herein is a kit comprising a detection complex comprising an antigen presenting molecule (APM) monomer or multimer capable of binding to an antigen to form an antigen-APM complex, wherein: the detection complex is conjugated to a reporter oligonucleotide, and the reporter oligonucleotide corresponds to the antigen or a portion thereof or the antigen/APM combination in the antigen-APM complex. In some embodiments, the detection complex comprises a scaffold conjugated to multiple molecules of the APM. In some embodiments, the scaffold is conjugated to the reporter oligonucleotide. In any of the embodiments herein, the kit can further comprise a probe or probe set configured to hybridize to the reporter oligonucleotide. In any of the embodiments herein, the kit can further comprise the antigen.

In some embodiments, disclosed herein is a kit comprising a plurality of detection complexes, wherein: each detection complex comprises a multimer of an antigen presenting molecule (APM) capable of binding to an antigen to form an antigen-APM complex, and each detection complex is conjugated to a reporter oligonucleotide corresponding to the antigen or a portion thereof or the antigen-APM complex. In some embodiments, two or more or all of the plurality of detection complexes comprise molecules of the same APM. In some embodiments, two or more or all of the plurality of detection complexes comprise molecules of different APM types. In any of the embodiments herein, the kit can further comprise a plurality of antigens, wherein each antigen comprises an antigen peptide capable of binding to a molecule of the APM in one of the plurality of detection complexes. In some embodiments, the plurality of antigens are selected from the group consisting of tumor associated antigens, tumor specific antigens, neoantigens, autoantigens, infectious agents, toxins, allergens, haptens, or a combination thereof.

In some aspects, provided herein is a kit, comprising: a) an antigen; b) a detection complex comprising an antigen presenting molecule (APM) monomer or multimer capable of binding to the antigen to form an antigen-loaded detection complex, wherein: the antigen and/or the detection complex is conjugated to a reporter oligonucleotide, the reporter oligonucleotide corresponds to the antigen or a portion thereof or the antigen/APM combination in the antigen-loaded detection complex, and the antigen-loaded detection complex is capable of binding to a cell receptor; c) a probe or probe set capable of hybridizing to the reporter oligonucleotide. In some embodiments, the probe or probe set is not immobilized on a planar substrate.

In some aspects, provided herein is a kit, comprising: a) a plurality of peptide-loaded detection complexes, wherein: each peptide-loaded detection complex can comprise an MHC multimer and molecules of an antigen peptide bound to the MHC molecules of the multimer, wherein the MHC multimer can comprise a scaffold conjugated to a reporter oligonucleotide comprising a barcode region corresponding to the antigen peptide or a portion thereof or the antigen peptide/MHC combination in the peptide-loaded detection complex, and the plurality of peptide-loaded detection complexes comprise antigen peptide/MHC complexes capable of binding to T cell receptors of different antigen specificities; and b) a plurality of probes or probe sets each capable of hybridizing to the barcode region or a portion thereof in a particular reporter oligonucleotide of the plurality of peptide-loaded detection complexes. In some embodiments, the plurality of probes or probe sets are circular or circularizable upon hybridization to the reporter oligonucleotides of the plurality of peptide-loaded detection complexes. In some embodiments, the plurality of probes or probe sets are not immobilized on a planar substrate.

In some aspects, provided herein is a kit, comprising: a) a plurality of peptide-loaded detection complexes, wherein: each peptide-loaded detection complex comprises an MHC multimer and molecules of an antigen peptide bound to the MHC molecules of the multimer, wherein the MHC multimer comprises a scaffold conjugated to a reporter oligonucleotide, the reporter oligonucleotide comprises i) a barcode region corresponding to the antigen peptide or a portion thereof or the antigen peptide/MHC combination in the peptide-loaded detection complex, and ii) a capture region, and the plurality of peptide-loaded detection complexes comprise antigen peptide/MHC complexes capable of binding to T cell receptors of different antigen specificities; and b) a substrate comprising a plurality of capture agents each comprising: i) a capture domain configured to bind to the capture region, and ii) a spatial barcode corresponding to the location of the capture agent on the substrate. In some embodiments, the kit further comprises: c) one or more reagents for generating a spatially labeled polynucleotide comprising (i) a sequence of the reporter oligonucleotide or complement thereof and (ii) a sequence of the spatial barcode or complement thereof. In some embodiments, the substrate is a planar substrate.

### BRIEF DESCRIPTION OF THE DRAWINGS

The following drawings illustrate certain embodiments of the features and advantages of this disclosure. These embodiments are not intended to limit the scope of the appended claims in any manner.

FIG. 1 depicts an exemplary antigen-loaded detection complex.

FIG. 2 illustrates an exemplary method using MHC tetramers (tetramer 1 and tetramer 2) loaded with different antigen peptides and conjugated to different corresponding reporter oligonucleotides (reporter oligonucleotide 1 and reporter oligonucleotide 2, respectively).

FIGS. 3A-3B depict exemplary methods of detecting antigen-loaded detection complexes bound to receptors (e.g., TCRs) in a biological sample in situ.

FIG. 4 depicts an exemplary array comprising spatially barcoded capture agents for detecting antigen-loaded detection complexes bound to receptors (e.g., TCRs) in a biological sample.

### DETAILED DESCRIPTION

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Detecting antigen and receptor interactions in a biological sample remains challenging. For instance, due to the weak binding of antigens and/or antigen presenting molecules to their receptors inside tissue sections, detecting these interactions can suffer from low sensitivity, which is often exacerbated by high background or noise. In particular, sensitive and multiplex methods for detecting different populations of T cell receptors (TCRs) are still needed. To address the weak binding of MHC molecules to the TCRs, concatemerized MHC complexes can be used to bind TCRs in vitro with multiple MHC complexes, which enhances the avidity to TCRs. The TCRs must be intact and functional for the binding between MHC and TCR to occur. However, most in situ assays require tissue fixation with formaldehyde or other crosslinkers that would crosslink lipids and proteins including TCRs inside a cell or tissue sample, obscuring TCRs such that they cannot bind with MHCs or pMHC complexes. Furthermore, methods that rely on antibodies for detecting pMHC/TCR binding are generally low in plexity and are of limited use due to the potentially very large diversity of antigen receptors in a biological sample (e.g., a tissue section).

In some embodiments, provided herein is a method for in situ detection of a receptor on and/or in a cell at a location in a biological sample using a ligand-loaded detection complex, such as detection of TCRs in situ in a cell or tissue sample using pMHC detection complexes. In some embodiments, the biological sample is a cell or tissue sample on a substrate (e.g., a slide or slip), such as a tissue section. In some embodiments, provided herein is a method comprising contacting a cell or tissue sample (e.g., a tissue section) on a slide or slip with an antigen and a detection complex simultaneously or in any order, wherein: cells or nuclei in the biological sample are not partitioned in individual partitions (e.g., emulsion droplets or microwells), the detection complex comprises an antigen-presenting molecule (APM) monomer or multimer that binds to the antigen to form an antigen-loaded detection complex, wherein the antigen-loaded detection complex is conjugated to a reporter oligonucleotide, the reporter oligonucleotide comprises a barcode region for identifying the antigen or a portion thereof or the antigen/APM combination in the antigen-loaded detection complex, and the antigen-loaded detection complex binds to a receptor on and/or in a cell at a location in the cell or tissue sample; optionally fixing and/or crosslinking the cell or tissue sample, e.g., to fix and/or crosslink the antigen-loaded detection complex with the bound receptor; contacting the cell or tissue sample with a probe or probe set that hybridizes to the reporter oligonucleotide; and imaging the slide or slip having the cell or tissue sample thereon to detect a signal associated with the probe or probe set or a product thereof at the location in the cell or tissue sample. In some embodiments, the biological sample is fixed, crosslinked, de-crosslinked, and/or cleared, and then contacted with an antigen and a detection complex, e.g., with a detection complex that is pre-loaded with an antigen. In some embodiments, the biological sample is not fixed, crosslinked, de-crosslinked, or cleared prior to contacting with an antigen, a detection complex, or a detection complex pre-loaded with an antigen.

In some embodiments, provided herein is a method for detection of a receptor on and/or in a cell at a location in a biological sample, comprising contacting the sample with a ligand-loaded detection complex that binds to the receptor, followed by capturing a reporter oligonucleotide of the ligand-loaded detection complex on a substrate (e.g., a slide or slip). In some embodiments, the biological sample is a cell or tissue sample, such as a tissue section. In some embodiments, provided herein is a method comprising contacting a cell or tissue sample with an antigen and a detection complex simultaneously or in any order, wherein: the detection complex comprises an antigen-presenting molecule (APM) monomer or multimer that binds to the antigen to form an antigen-loaded detection complex, wherein the antigen-loaded detection complex is conjugated to a reporter oligonucleotide, the reporter oligonucleotide comprises a barcode region for identifying the antigen or a portion thereof or the antigen/APM combination in the antigen-loaded detection complex and comprises a capture region, and the antigen-loaded detection complex binds to a receptor on and/or in a cell at a location in the cell or tissue sample; capturing the reporter oligonucleotide or a portion thereof by a capture agent, where in the capture agent is at a location on a slide or slip and comprises: i) a capture domain that binds to the capture region, and ii) a spatial barcode corresponding to the location of the capture agent on the slide or slip and the corresponding location in the cell or tissue sample; generating a spatially labeled polynucleotide comprising (i) a sequence of the reporter oligonucleotide or complement thereof and (ii) a sequence of the spatial barcode or complement thereof; and determining a sequence of the spatially labeled polynucleotide to detect the spatial barcode and the reporter oligonucleotide. In some embodiments, the determined sequences of the spatial barcode and the reporter oligonucleotide are used to identify the corresponding antigen or antigen/APM combination at the location in the cell or tissue sample. In some embodiments, the method does not comprise imaging the biological sample (e.g., using fluorescent microscopy) to detect the sequence of the reporter oligonucleotide or complement thereof and/or the sequence of the spatial barcode or complement thereof.

Provided herein in some aspects are in situ platforms and spatial arrays for analyzing clonal T cell populations, TCRs, and antigen binding specificities of TCRs of cells in a cell or tissue sample to cognate MHC molecules using antigen-loaded detection complexes (e.g., pMHC tetramers as labelling agents). The antigen-loaded detection complexes disclosed herein can be used to detect the locations of T cells in a biological sample, for example, in tissues such as tumors comprising infiltrating immune cells. In some embodiments, the T cells comprise known antigen-responsive T cells. In some embodiments, the TCR profile of T cells in the biological sample can be detected and their spatial distribution revealed. The methods disclosed herein may be applied at high-throughput to detect T cells, their TCR transcripts, and/or their antigen binding specificities, and reveal their spatial relationship with respect to one or more features and/or other molecules in the biological sample. The TCR repertoire or TCR profile as well as the spatial distribution of the antigen binding specificities can be analyzed in situ in the biological sample and/or using an array comprising spatially barcoded capture agents.

In some embodiments, the biological sample, such as a tissue section with T cells, can be mildly crosslinked or embedded with matrix. In some aspects, the matrix crosslinks to itself but not to biological components. In some embodiments, the antigen-loaded detection complexes comprising reporter oligonucleotides (e.g., loaded tetramers with DNA barcodes) are contacted to T cells in the tissue section. In some embodiments, the antigen-loaded detection complexes (e.g., loaded tetramers with detectable barcodes) are allowed to bind to T cells with different T cell receptors (TCRs).

In some embodiments, tissue embedding is performed. In some embodiments, tissue embedding is performed without crosslinking proteins. In some aspects, fresh cut tissue sections with T cells comprising different active T cell receptors are contacted with antigen-loaded detection complexes (e.g., loaded-tetramers). T cell receptors in unfixed tissues are active, flexible, and able to move around/rotate freely. In some aspects, synthetic crosslinking agents that do not crosslink the biological material are applied. In some embodiments, the biological sample is contacted with a first matrix-forming material. In some embodiments, a first polymerized matrix is formed from the first matrix-forming material, thereby embedding the biological sample in the first polymerized matrix. In some aspects, the crosslinkers are crosslinked in the tissue section forming a matrix without crosslinking to the TCRs. The TCRs are thus left intact and active. In some embodiments, the first matrix-forming material does not react with biological molecules in the biological sample. In some embodiments, protein molecules in the biological sample are not crosslinked by the first matrix-forming material or to the first polymerized matrix, and wherein the TCR in the biological sample embedded in the first polymerized matrix remains capable of binding to the MHC monomer or multimer of the peptide-loaded detection complex. The TCRs in the crosslinked tissue are still active as they are not crosslinked.

In some aspects, provided herein are detection complexes (e.g., barcoded MHC tetramers loaded with peptides) for screening T cell populations and to identify cells with pMHC-reactive T cell receptors. In some embodiments, the methods provided herein comprise contacting a plurality of peptide-loaded detection complexes with a biological sample, thereby allowing binding of the peptide-loaded detection complexes to TCRs at one or more locations in the biological sample. In some embodiments, a detection complex disclosed herein comprises a reporter oligonucleotide that corresponds to an antigen (e.g., a peptide) or an antigen/MHC complex (e.g., a pMHC complex), and signals associated with the reporter oligonucleotide detected at a particular location (e.g., in sequential probe hybridization cycles) can indicate the presence/absence or a level of the detection complex and TCR(s) with the corresponding antigen binding specificity at the particular location in the biological sample. In some embodiments, a probe or probe set that directly or indirectly binds to the reporter oligonucleotide can be used to detect the reporter oligonucleotide and the corresponding antigen binding specificity in situ. In some embodiments, a capture agent on a substrate can be used to capture the reporter oligonucleotide, a product of the reporter oligonucleotide, a probe that binds to the reporter oligonucleotide, and/or a product of the probe, in order to generate a spatially barcoded polynucleotide which can be analyzed (e.g., using nucleic acid sequencing) for revealing spatial information of antigen binding specificities in the biological sample.

In some embodiments, a reporter oligonucleotide disclosed herein can comprise one or more barcode sequences. In some embodiments, a reporter oligonucleotide disclosed herein can comprise a region that directly or indirectly binds to a probe comprising one or more barcode sequences (e.g., the probe is a barcoded probe), and the reporter oligonucleotide can be detected by targeting the barcode sequence(s) in the probe or a product thereof.

In some embodiments, the detection complexes can be applied to a tissue section, such as a tumor section with infiltrated immune cells. The tissue section may but does not need to be permeabilized to allow detection complexes binding to cell surface receptors. In some embodiments, the detection complexes are allowed to interact with the T cell receptors of T cell populations in the tissue sections. In some aspects, excess detection complexes (e.g., unbound or non-specifically bound pMHC tetramers) are removed from the tissue sample, such as that the detection complexes which remain in the sample are specifically bound to TCRs.

In some aspects, the tissue sample can be permeabilized and/or lysed, such that the detection complexes specifically bound to TCRs can be released and/or migrated onto an array surface for capture and spatial barcoding. In some aspects, the reporter oligonucleotide, a product of the reporter oligonucleotide, a barcoded probe targeting the reporter oligonucleotide, and/or a product of the barcoded probe can be released and/or migrated onto the spatial array for capture and spatial barcoding. In some aspects, the reporter oligonucleotide and/or a barcoded probe targeting the reporter oligonucleotide can comprise features (e.g., capture regions) that can be captured on a spatial array, such that a spatial barcode can be assigned to the reporter oligonucleotide, the barcoded probe, and/or a product thereof for next generation sequencing (NGS) read outs. In some embodiments, a spatially barcoded oligonucleotide comprising a barcode sequence (e.g., for identifying an antigen peptide or a pMHC complex) and a spatial barcode can be generated on the array, pooled, and sequenced.

In some aspects, the reporter oligonucleotide and/or a barcoded probe targeting the reporter oligonucleotide can be detected by detectable probes that directly or indirectly generate detectable signals in situ. In some aspects, the tissue sample can be subjected to an in situ read out using detectable probes or probe sets added to the sample. The probes or probe sets can target one or more barcode sequences in the detection complexes. In some embodiments, the reporter oligonucleotide comprises one or more barcode regions comprising one or more barcode sequences. In some embodiments, a barcode sequence in the reporter oligonucleotide corresponds to the antigen loaded onto the detection complex or to the antigen/antigen presenting molecule combination. In some embodiments, the probes or probe sets hybridize to the barcode regions or complements thereof. The probes or probe sets can be circular probes or circularizable probes probe sets. In some embodiments, the probes or probe sets can be ligated. In some aspects, the probes or probe sets can be used to generate detectable signals that can be amplified via any suitable signal amplification technique. The signal amplification can comprise enzymatic amplification (e.g., generating a nucleic acid amplicon) and/or non-enzymatic amplification (e.g., forming a complex comprising multiple detectably labeled probes). The enzymatic amplification can include rolling circle amplification (RCA) and/or primer exchange reaction (PER). The non-enzymatic amplification can include chain reactions or branched hybridization reactions, such as hybridization chain reaction (HCR), branched DNA (bDNA), or a combination thereof. The barcode sequences or complements in the probes or products thereof (e.g., RCA products or probe hybridization complexes) can be read by applying combinatorial or sequential decoding techniques to determine the identity of the antigen-loaded detection complex (e.g., pMHC tetramer) at a given location.

An in situ platform and/or a spatial array platform can be used to screen a pool of antigen-loaded detection complexes (e.g., pMHC tetramers) in high multiplex.

In some embodiments, a population of dissociated cells can be contacted with an antigen and a detection complex simultaneously or in any order, wherein the detection complex comprises an antigen-presenting molecule (APM) monomer or multimer that binds to the antigen to form an antigen-loaded detection complex, wherein the antigen-loaded detection complex is conjugated to a reporter oligonucleotide; the reporter oligonucleotide corresponds to the antigen or a portion thereof or the antigen/APM combination in the antigen-loaded detection complex; and the antigen-loaded detection complex binds to a receptor on and/or in a cell of the population of dissociated cells to label the cell with the antigen-loaded detection complex. In some embodiments, the population of dissociated cells comprising the labeled cell can be deposited on a substrate to provide a deposited labeled cell, which can be contacted with a probe or probe set that hybridizes to the reporter oligonucleotide. In some embodiments, the method comprises detecting a signal associated with the probe or probe set or a product thereof at a location on the substrate, using an in situ platform. In some embodiments, the antigen-loaded detection complex comprises a sample-specific barcode sequence. In some embodiments, the reporter oligonucleotide conjugated to the antigen-loaded detection complex comprises a sample-specific barcode sequence. In some embodiments, the method comprises using an in situ platform to detect: i) a signal associated with the sample-specific barcode sequence (e.g., using a probe or probe set that hybridizes to the sample-specific barcode sequence) in a cell, thereby identifying the sample origin of the cell, as well as ii) a signal associated with the antigen-loaded detection complex in and/or on the cell, thereby identifying one or more antigen binding characteristics of the cell.

In some embodiments, a method disclosed herein may be used to locate known T cell populations in a sample, e.g., in a tumor. For instance, a multiplex pMHC tetramer panel can be designed based on the TCR sequences identified in cells of a sample, e.g., through single-cell RNA sequencing of TCR transcripts. The multiplex pMHC tetramer panel can be applied to a tissue section of the sample to locate T cells having the corresponding antigen binding specificities in the sample. In these examples, only those pMHC tetramers for TCRs that have been identified (e.g., in a single-cell in vitro screen) need to be applied to the tissue section of the sample, and the locations of the TCRs can be detected and identified using an in situ readout and/or a spatial array readout. In some embodiments, a method disclosed herein may be used to discover reactive T cells in a sample. In these examples, a multiplex pMHC tetramer panel can be designed without a prior knowledge of whether cells expressing a TCR having a particular antigen binding specificity exist in a sample. Again, the TCRs can be discovered and their locations in the sample can be revealed using an in situ readout and/or a spatial array readout.

Methods, compositions, kits, and systems for performing the in situ and/or spatial assays are provided. In some embodiments, the present disclosure provides methods for high-throughput profiling of a large number of clonal T cell populations comprising TCRs with varying antigenic specificities. The methods and compositions disclosed herein may be used in research, diagnostics, and drug target discovery. Identification and pairing of antigens with their receptors (e.g., pMHC with cognate TCRs of T cells) in various tissues could be used for development of therapeutic and/or prophylactic agents, e.g., TCR therapeutic treatment modalities and/or anti-disease vaccination.

### II. Detection Complexes

Provided herein in some aspects are detection complexes for detecting interactions between antigens and binding partners such as T cell receptors and antigen responsive T cells. In some embodiments, a biological sample (e.g., fresh or frozen tissue samples) is contacted with detection complexes each comprising an antigen presenting molecule monomer or multimer configured to bind to one or more molecules of an antigen to form an antigen-loaded detection complex. In some embodiments, the detection complex is conjugated to a reporter oligonucleotide, and the reporter oligonucleotide corresponds to the antigen or a portion thereof or the antigen/antigen presenting molecule combination in the antigen-loaded detection complex. In some embodiments, the antigen-loaded detection complex binds to a receptor on and/or in a cell (e.g., T cell receptor of a T cell) at one or more locations in the biological sample. In some embodiments, a probe or probe set that hybridizes to the reporter oligonucleotide can be contacted with the biological sample, and a signal associated with the probe or probe set or a product thereof can be detected at the one or more locations in the biological sample. In some embodiments, an array comprising spatially barcoded capture agents can be used to analyze the antigen-loaded detection complexes bound to receptors in the biological sample.

Disclosed herein in some aspects are detection complexes that comprise an antigen presenting molecule (APM) monomer or multimer. In some embodiments, the APM monomer or multimer binds to an antigen to form an antigen/APM complex, such that the detection complex becomes loaded with the antigen to form an antigen-loaded detection complex. In some embodiments, the antigen may be a peptide, lipid, small molecule, metabolite, phospholipid, or a fatty acid. An antigen presenting molecule (e.g., an MHC molecule) may be loaded with a peptide to form a peptide-MHC (pMHC) complex. In some embodiments, the antigen-loaded detection complex is conjugated to a reporter oligonucleotide. In some embodiments, the reporter oligonucleotide corresponds to the antigen or a portion thereof or the antigen/APM combination in the antigen-loaded detection complex. In some embodiments, the reporter oligonucleotide allows the detection of an interaction between the antigen or the antigen/APM complex and a cell receptor (e.g., T cell receptor) or an antigen responsive cell (e.g., an antigen peptide responsive T cell). In some embodiments, the antigen-loaded detection complex comprises a scaffold covalently or non-covalently conjugated to the APM monomer or multimer. In some embodiments, the antigen-loaded detection complex binds to a receptor on and/or in a cell at a location in the biological sample.

A detection complex disclosed herein may be provided in a loadable form which comprises one or more APM (e.g., MHC molecules) without a bound antigen, that is, the detection complex may comprise an empty APM (e.g., a peptide-free or “empty” loadable MHC molecule). In some embodiments, a detection complex disclosed herein is provided in a loaded form which comprises one or more bound molecules of the same antigen or different antigens. In some embodiments, a detection complex disclosed herein comprises one or more peptide-MHC (pMHC) complexes, such as one or more class I peptide-MHC complexes and/or one or more class II peptide-MHC complexes. In some embodiments, a multimer of peptide-free MHC molecules or “empty MHC molecules” can be contacted with one or more antigen peptides of the same or different sequences to generate an antigen loaded multimer, which can be used as a detection complex disclosed herein. In some embodiments, all of the MHC molecules of the multimer are loaded with antigen peptides of the same sequence. In some embodiments, two or more of the MHC molecules of the multimer are loaded with antigen peptides of different sequences.

In some embodiments, provided herein is a plurality (e.g., a panel) of loadable detection complexes (e.g., comprising peptide-free MHC molecules) for high throughput epitope identification and/or TCR specificity determination. In some embodiments, the plurality of loadable detection complexes are configured such that each detection complex is capable of binding to one or more molecules of an antigen, such an antigenic peptide, and collectively, the plurality of loadable detection complexes are configured to bind a plurality (e.g., a panel) of different antigenic peptides. In some embodiments, each of the different antigenic peptides is represented by or corresponds to one or more detectable labels such as one or more reporter oligonucleotide molecules coupled to each antigenic peptide/antigen presenting molecule via a scaffold. In some embodiments, the interactions between multiple TCRs (or antigenic peptide responsive T cells expressing the TCRs) and a library of antigenic peptides can be screened by detecting signals associated with the reporter oligonucleotides.

In some embodiments, a detection complex disclosed herein comprises a peptide-free MHC class I molecule comprising a heavy chain comprising an alpha-1 domain and an alpha-2 domain connected by a disulfide bridge, with one or more cysteine residues artificially introduced in the alpha-1 domain and/or one or more cysteine residues artificially introduced in the alpha-2 domain. In some embodiments, the MHC class I molecule comprises a mutant cysteine residue positioned at amino acid residue 84 or 85 in the alpha-1 domain and/or a mutant cysteine residue positioned at amino acid residue 139 in the alpha-2 domain. In some embodiments, a detection complex disclosed herein comprises a disulfide stabilized version of a MHC class I molecule that can be prepared without antigenic peptide bound to the peptide binding cleft of the MHC molecule. Exemplary peptide-free MHC class I molecules that can be used in the present disclosure include those described in WO 2020/064915 (U.S. Ser. No. 17/279,025), which is incorporated herein by reference.

In some embodiments, a detection complex disclosed herein allows detection of interaction between an antigen (e.g., an antigenic peptide loaded to antigen presenting molecule(s) of the detection complex) and a receptor (e.g., TCR) or a cell expressing the receptor (e.g., an antigenic peptide responsive T cell). In some embodiments, a detection complex disclosed herein can be utilized for (i) identifying one or more cells expressing the receptor in a population of cells in a tissue sample, and/or (ii) TCR fingerprinting/profiling by screening of TCR-pMHC interactions using large libraries of antigen-specific detection complexes.

In some embodiments, a detection complex disclosed herein comprises a linker and/or a scaffold to which is attached one or more antigen presenting molecules and one or more reporter oligonucleotide molecules. In some embodiments, the detection complex comprises more than one antigen presenting molecule and is designated as a multimer. In some embodiments, the scaffold is conjugated with a plurality of streptavidin molecules and a plurality of reporter oligonucleotide molecules. In some embodiments, the scaffold comprises a streptavidin tetramer configured to bind four biotin moieties each coupled to an MHC molecule (e.g., via a linker), thereby forming an MHC tetramer. In some embodiments, the scaffold comprises a polysaccharide (e.g., dextran) to which are attached MHC molecules either directly or via linkers.

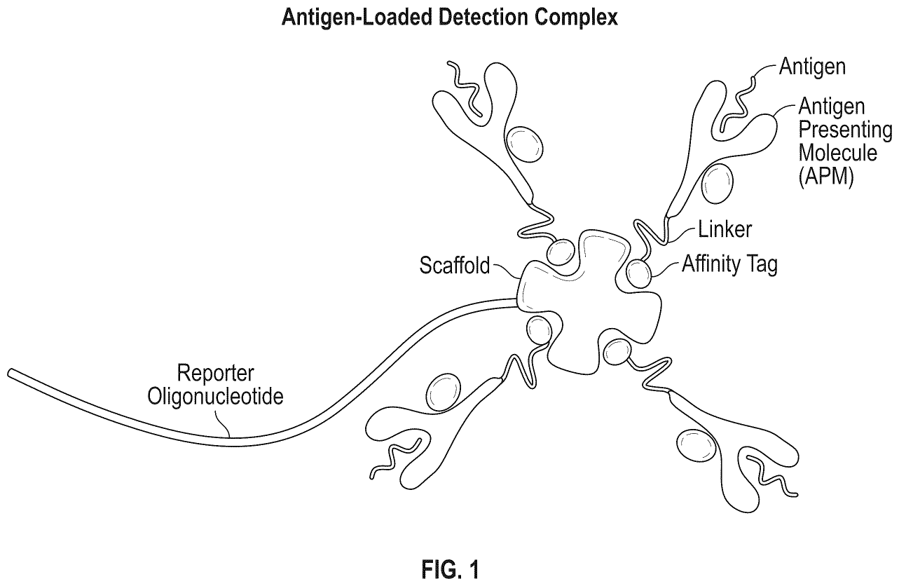

FIG. 1 depicts an exemplary antigen-loaded detection complex comprising: i) an antigen presenting molecule (e.g., an MHC Class I molecule) bound to an antigen (e.g., a peptide), ii) a reporter oligonucleotide, and iii) a scaffold coupling the antigen presenting molecule and the reporter oligonucleotide. In some cases, the antigen presenting molecule can be coupled to the scaffold via a linker comprising an affinity tag that binds to the scaffold. An exemplary antigen-loaded detection complex can be a tetramer comprising four antigen/antigen presenting molecule complexes (e.g., four pMHC complexes).

Any suitable techniques of loading antigens (e.g., peptides) onto the antigen presenting molecules (e.g., MHC molecules) may be utilized, for instance, as described in WO 2020/064915 (U.S. Ser. No. 17/279,025) and US 2021/0139985, which are incorporated herein by reference.

### A) Antigen Presenting Molecules

Disclosed herein are antigen presenting molecule (APM) monomers and multimers that bind to antigens to form antigen-loaded antigen presenting complexes. An APM may be any molecule on the surface or inside of antigen presenting cells that can be bound to an antigen or portion thereof and further present the antigen to cells. Presentation of antigens or portions thereof to cells (e.g., T cells) may stimulate an immune response. Exemplary antigen presenting molecules include but are not limited to major histocompatibility complexes (e.g., MHC Class I or MHC Class II), non-classical MHCs, MHC-like molecules.

In some aspects, the antigen presenting molecules are major histocompatibility complex (MHC) molecules (e.g., MHC class I or class II), MHC-like molecules, non-classical MHC molecules (e.g., MR1), or a combination thereof.

In some embodiments, the detection complex comprises an antigen presenting molecule (APM) monomer. For instance, the detection complex comprises one molecule of an MHC. In some aspects, the one molecule of MHC is pre-loaded with an antigen (e.g., antigen peptide). In some aspects, the one molecule of MHC is not pre-loaded with an antigen (e.g., antigen-free antigen presenting molecule, such as antigen-free MHC). In some aspects, the antigen-free MHC molecule is contacted with an antigen to form an antigen-loaded detection complex. In some embodiments, an “empty MHC molecules” that can be contacted with one or more antigen peptides is conjugated to a reporter oligonucleotide.

In some embodiments, the antigen presenting molecule comprises an antigen presenting molecule (APM) multimer. In some embodiments, the antigen presenting molecule multimer comprises 2, 3, 4, 5, 6, 7, 8, 9, 10, or more APM monomers (e.g., MHC molecules). In some embodiments, the antigen-loaded detection complex comprises 2, 3, 4, 5, 6, 7, 8, 9, 10, or more molecules of the antigen. In some embodiments, the antigen-loaded detection complex comprises a dimer, trimer, tetramer, pentamer, hexamer, heptamer, octamer, streptamer, or dodecamer of peptide-MHC monomers. In some embodiments, the antigen-loaded detection complex comprises a dimer, tetramer, pentamer, octamer, streptamer, or dodecamer of peptide-MHC monomers. In some aspects, the antigen presenting molecule multimer is not pre-loaded with antigens (e.g., antigen-free MHC or antigen-free multimer). The antigen presenting molecule multimer may be contacted with antigens to form an antigen-loaded detection complex. In some aspects, the antigen presenting molecule multimer is pre-loaded with antigens. In some embodiments, each monomer of the antigen presenting molecule multimer may be loaded with an antigen. In some aspects, one or more monomers of the antigen presenting molecule multimer is not loaded with an antigen.

The MHC molecules can bind antigenic peptides derived from pathogens and display them on the cell surface for recognition by the appropriate cells (e.g., T cells). In some embodiments, the MHC molecules may bind peptides in an intracellular processing compartment and present these peptides on the surface of antigen presenting cells to T cells. In cells, class I MHC proteins typically present antigenic peptides derived from proteins actively synthesized in the cytoplasm of the cell. In contrast, class II MHC proteins typically present antigenic peptides derived either from exogenous proteins that enter a cell's endocytic pathway or from proteins synthesized in the ER. Intracellular trafficking permits an antigenic peptide to become associated with an MHC protein. The resulting MHC-peptide complex then travels to the surface of the cell where it is available for interaction with a TCR.

In some embodiments, an MHC molecule disclosed herein can bind to a peptide comprising an epitope, which is an antigenic determinant recognized by a receptor such as a TCR of a T cell. In some embodiments, an MHC molecule can bind to a peptide comprising a T cell epitope. In some embodiments, the interaction of the peptide-MHC with a TCR leads to expansion and functional stimulation of the specific T cells in a peptide-MHC-directed fashion.

In some embodiments, a plurality (e.g., a panel) of loadable detection complexes disclosed herein comprises MHC molecules of the same type. In some embodiments, a plurality (e.g., a panel) of loadable detection complexes disclosed herein comprises MHC molecules of one or more different types.

MHC-restricted antigen recognition, or MHC restriction, refers to the fact that a T cell can interact with a self-MHC molecule and a foreign peptide bound to it, but will only respond to the antigen when it is bound to a particular type of MHC molecule. In humans, the MHC is encoded by the human leukocyte antigen (HLA) gene complex. Thus, in the present context, the term “MHC” also encompasses “HLA”. There exist three major types of HLA and therefore MHC in the present context include, but are not limited to, HLA alleles that are coded in the gene loci for HLA-A, HLA-B, and HLA-C. Similarly, MHC include, but are not limited to, MHC class I-like molecules such as HLA-DP, HLA-DM, MHC Class II molecules such as HLA-DR, HLA-DP, and HLA-DQ, or HLA-DOA, HLA-DOB, HLA-E, HLA-F, HLA-G, HLA-H, MIC A, MIC B, CD1d, ULBP-1, ULBP-2, and ULBP-3. In some embodiments, an MHC disclosed herein is of the HLA-A1, HLA-A2, HLA-A3, HLA-A23, HLA-A24, HLA-A26, HLA-A29, HLA-A30, HLA-B7, HLA-B8, HLA-B27, HLA-B35, HLA-B41, HLA-B44, HLA-B57, or HLA-B58 type. In some embodiments, an MHC disclosed herein is of the H-2Kb, HLA-A*01:01, HLA-A*02:01, HLA-A*03:01, HLA-A*11:01, HLA-A*24:02, HLA-A*29:02, HLA-A*32:01, HLA-B*07:02, HLA-B*08:01, HLA-B*13:02, HLA-B*14:02, HLA-B*15:01, HLA-B*18:01, HLA-B*27:05, HLA-B*35:01, HLA-B*40:01, HLA-B*44:02, HLA-B*44:03, or HLA-B*57:01 type. In some embodiments, a plurality (e.g., a panel) of loadable detection complexes disclosed herein comprises MHC molecules of the H-2Kb, HLA-A*01:01, HLA-A*02:01, HLA-A*03:01, HLA-B*07:02, HLA-B*08:01, HLA-A*11:01, or HLA-A*24:02 type. In some embodiments, loadable detection complexes disclosed herein may comprise MHC molecules of the HLA types disclosed in U.S. Pat. No. 7,524,503 and US 2020/0055918, which are incorporated herein by reference.

An MEW class I protein is an integral membrane protein comprising a glycoprotein heavy chain (α chain), which has three extracellular domains (α1, α2 and α3), a transmembrane domain, and a cytoplasmic domain. An MEW class I α chain (or class I heavy chain) can be any naturally occurring polypeptide, or one encoded by an artificially mutated a chain gene, essentially corresponding to at least the α1 and α2 domains of one of the gene products of an MEW class I α gene (e.g. HLA-A, HLA-B or HLA-C gene). The transmembrane and cytoplasmic domains may be omitted while an MEW class I α chain retains biological activity. An MHC class I α chain can be any variant with and without the usual glycosylation of the α2 domain, or any allelic variant of a class I α gene, as well as any equivalents, including those which may be produced synthetically or recombinantly by, for example, site-directed mutagenesis of a naturally occurring variant. An MEW class I molecule can be a covalently or non-covalently joined complex of an MEW class I α chain and a soluble subunit called the β2-microglobulin chain (also known as the class I light chain, or the class I β chain). A class I β chain can be any naturally occurring polypeptide, or one encoded by an artificially mutated β2-microglobulin gene, essentially corresponding to the gene product of a β2-microglobulin gene. A class I β chain can be any allelic variants of β2-microglobulin, as well as any equivalents, including those which may be produced synthetically or recombinantly by, for example, site-directed mutagenesis of a naturally occurring variant.

An MEW class II protein is a heterodimeric integral membrane protein comprising one α chain and one β chain. The α chain has two extracellular domains (α1 and α2), a transmembrane domain, and a cytoplasmic domain. The (3 chain contains two extracellular domains (β1 and β2), a transmembrane domain, and a cytoplasmic domain. An MEW class II α chain (or class II heavy chain) can be any naturally occurring polypeptide, or one encoded by an artificially mutated a gene, essentially corresponding to at least the α1 and α2 extracellular domains of one of the gene products of an MEW class II α gene. The transmembrane and cytoplasmic domains may be omitted while an MEW class II α chain retains biological activity. An MEW class II α chain can be any variant with and without the usual glycosylation of the α1 and α2 domains, or any allelic variant of a class II α gene, as well as any equivalents, including those which may be produced synthetically or recombinantly by, for example, site-directed mutagenesis of a naturally occurring variant. An MEW class II molecule can be a covalently or non-covalently joined complex of an MEW class II α chain and an MHC class II β chain (also known as the class II light chain, or the class II β chain). A class II β chain can be any naturally occurring polypeptide, or one encoded by an artificially mutated class II β gene, essentially corresponding to at least the β1 and β2 extracellular domain of one of the gene products of an MHC class II β gene. The transmembrane and cytoplasmic domains may be omitted while an MHC class II β chain retains biological activity. An WIC class II β chain can be any variant with and without the usual glycosylation of the β1 domain, or any allelic variant of a class II β gene, as well as any equivalents, including those which may be produced synthetically or recombinantly by, for example, site-directed mutagenesis of a naturally occurring variant.

The terms “MHC-peptide complex,” “MHC-peptide molecule,” “peptide-MHC complex,” “peptide-MHC molecule,” and “pMHC” are used interchangeably. Any portion of an MHC protein that forms a functional peptide binding groove and that has a peptide bound to the peptide binding groove can be referred to as the peptide-MHC complex. The terms “binding site,” “binding groove” and “binding domain” of an MHC molecule are used interchangeably unless specified otherwise. The domain organization of class I and class II molecules forms the antigen binding site, or peptide binding groove. A peptide binding groove refers to a portion of an MHC protein that forms a cavity in which a peptide can bind. According to the present disclosure, “a portion” of an MHC chain refers to any portion of an MHC chain that is sufficient to form a peptide binding groove upon association with a sufficient portion of another chain of an MHC protein. The conformation of a peptide binding groove is capable of being altered upon binding of an antigenic peptide to enable proper alignment of amino acid residues important for T cell receptor (TCR) binding to the MHC protein and/or peptide.

An MHC class I binding domain (or groove) is formed primarily by the α1 and α2 domains of an MHC class I α chain. In a preferred embodiment, an MHC class I binding domain includes the α3 domain of an α chain and β2-microglobulin, which may function to stabilize the overall structure of the MHC class I molecule. An MHC class I binding domain may also be essentially defined as the extracellular domain of an MHC class I molecule. In certain aspects, a portion of the extracellular domain may be omitted while retaining biological activity. For most MHC class I molecules, interaction of the α and β chains can occur in the absence of a peptide. However, the two chain complex of MHC class I is inherently unstable until the binding groove is filled with a peptide.

A peptide binding groove of a class II MHC protein can comprise portions of the α1 and β1 domains. In some embodiments, an MHC class II binding domain minimally includes the α1 and β1 domains. In some embodiments, an MHC class II binding domain includes the α2 and β2 domains, which are believed to stabilize the overall structure of the MHC binding cleft. An MHC class II binding domain may also be essentially defined as the extracellular domain of an MHC class II molecule. In certain aspects, a portion of the extracellular domain of an MHC class II molecule may be omitted while retaining biological activity, including antigen presentation.

Major Histocompatibility Complex Class III (MHC Class III) molecules are encoded by numerous genes, some of which are related to the immune system. MHC Class III molecules include complement proteins (C2, C4a, C4b, and Bf), cytokines (TNFα, TNFβ, and lymphotoxin), enzymes required for steroid synthesis, heat shock proteins and many unidentified proteins. Some MHC Class III molecules are important in immune regulation and inflammation. In some embodiments, MHC Class III molecules, including those that do not participate in binding antigenic peptides, may be used to detect an interacting partner by binding to the interacting partner (e.g., a specific binder) in a sample. For example, a probe or probe set can hybridize to a reporter oligonucleotide directly or indirectly conjugated to the MHC Class III molecule, and a signal associated with the probe or probe set or a product thereof can be detected at one or more locations in the sample, thereby detecting the interacting partner in situ. Alternatively, the reporter oligonucleotide can be captured on a spatial array and analyzed (e.g., using nucleic acid sequencing) to detect the spatial location of the MHC Class III molecule and its interacting partner in the sample.

In some embodiments, the antigen presenting molecule comprises a non-classical MHC molecule. Non-classical major histocompatibility complex (MHC) molecules are structurally similar to the classical MHC Class I molecules. Non-classical MHC molecules generally display limited or no polymorphism and their expression may be more tissue-restricted. Non-classical MHC molecules generate inhibitory or activating stimuli in natural killer (NK) cells. Non-classical class I molecules include but are not limited to HLA-E, F, G, HFE, CD1, MR1, Qa-1b, zinc-α2-glycoprotein (ZAG), Endothelial Protein C Receptor (EPCR). Non-classical class II molecules, include HLA-DM and HLA-DO, are non-peptide binding class II MHC-II homologs. In some embodiments, the non-classical MHC molecule disclosed herein interacts with either one or more T cells or NK cells. In some embodiments, the non-classical MHC molecule disclosed herein form a heterodimer with β2-microgloubulin (β2m).

In some aspects, the non-classical MHCs comprise CD1 proteins. CD1 proteins are a family of evolutionarily conserved class I molecules that present lipids for immune surveillance. In some aspects, the CD1 proteins include CD1a, CD1b, CD1c, CD1d. In some embodiments disclosed herein, the antigen comprises a lipid antigen. In some aspects, lipids include, but are not limited to mycolyl lipids, sulfolipids, mycoketides, and phospholipids.

In some embodiments, the antigen presenting molecule binds to a lipid to form a lipid-loaded detection complex. In some embodiments, the lipid loaded detection complex can bind to a receptor on and/or in a cell at a location in the biological sample. In some aspects, the non-classical MHCs comprise MHC class related protein (MR1) molecules. In some aspects, MR1 molecules can bind to a small molecule, metabolite, phospholipid, or a fatty acid, including but not limited to small organic compounds, biosynthetic intermediates, and chemical metabolites of vitamins.

In some embodiments, the antigen presenting molecule comprises an MHC-like molecule. MHC-like molecules are a class of cell surface receptors which interact with CD8 T cells, NK cells, or γδ T cells. Exemplary MHC-like molecules include T10, T22, FcRn, as well as stress-induced molecules including, MHC I chain-related protein A (MICA), MHC I chain-related protein B (MICAB), ULBP, Rae1⁺, and H60 molecules.

### B) Reporter Oligonucleotides

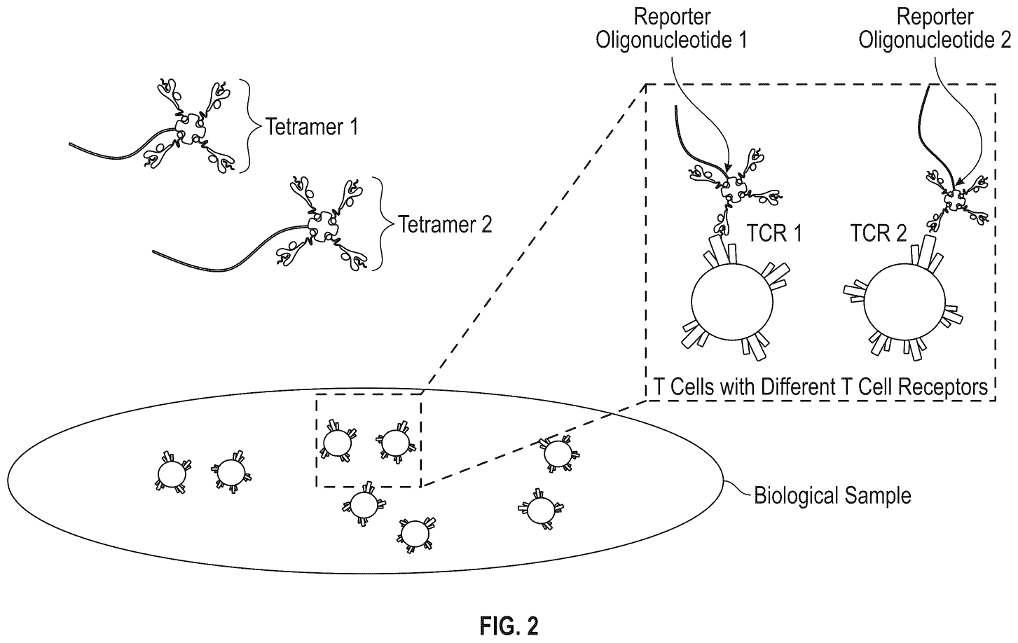

In the present disclosure, a detection complex comprises an antigen presenting molecule monomer or multimer and a reporter oligonucleotide. In some embodiments, the reporter oligonucleotide corresponds to the antigen or portion thereof bound to the antigen presenting molecule (e.g., MHC Class I) of the detection complex. For example, FIG. 2 illustrates an exemplary method comprising contacting a biological sample with two detection complexes e.g., MHC tetramers (tetramer 1 and tetramer 2), loaded with different antigen peptides and conjugated to different corresponding reporter oligonucleotides (reporter oligonucleotide 1 and reporter oligonucleotide 2, respectively). The biological sample may comprise clonal populations of T cells expressing T cell receptors (TCRs) of varying antigen specificities (e.g., TCR 1 and TCR 2, respectively). Contacting the antigen peptide-loaded tetramers to the TCRs facilitates engagement of pMFIC complexes to their cognate TCRs.

The reporter oligonucleotide may be single-stranded DNA (ssDNA) or single-stranded RNA (ssRNA). The reporter oligonucleotide may be linearly or circular, or may comprise both linear and circular segments.

In some embodiments, the reporter oligonucleotide is covalently or non-covalently conjugated to the antigen. Methods of conjugating the reporter oligonucleotide to the antigen include but are not limited to those described in U.S. Pat. Nos. 11,231,419 and 11,092,601, which are incorporated herein by reference. In some embodiments, the reporter oligonucleotide is covalently or non-covalently conjugated to the MEW, optionally wherein the conjugation is via one or more linkers.

In some embodiments, the reporter oligonucleotide is covalently or non-covalently conjugated to a scaffold of the antigen-loaded detection complex (e.g., peptide-loaded detection complex). In some embodiments, the scaffold is in turn covalently or non-covalently conjugated to one or more antigen presenting molecules such as MEW molecules. In some embodiments, the conjugation between the reporter oligonucleotide and the scaffold and/or the conjugation between the scaffold and the one or more antigen presenting molecules are via one or more linkers.

In some embodiments, the scaffold comprises a polysaccharide such as dextran. Methods of conjugating the reporter oligonucleotide to the antigen include but are not limited to those described in US 2017/0343545 and US 2018/0180601, which are incorporated herein by reference.

The reporter oligonucleotide, the scaffold, and/or the linker optionally further comprise binding pairs, such as streptavidin or avidin and biotin, for coupling the reporter oligonucleotide to the scaffold, optionally via the linker. In some embodiments, the scaffold comprises a streptavidin, a avidin, or mutant, variant, or analog thereof.

In some aspects, the detection complex may comprise at least one reporter oligonucleotide, such as two, three, four, five, six, seven or eight reporter oligonucleotides. The reporter oligonucleotides may be the same or different in each detection complex. Thus, the antigen peptide may be represented by one multiple-conjugated detection complex (e.g., two reporter oligonucleotides comprising two unique barcode sequences). Thus, in some aspects, one antigen peptide is represented by at least two different reporter oligonucleotides, and binding of the detection complex to one or more antigen peptide responsive T cells is detected by detecting the presence of the at least two different reporter oligonucleotides bound to an antigen responsive T cell through the detection complexes. In some embodiments, the peptide-loaded detection complex may but does not need to be fluorescent or fluorescently labeled, and labeling may be achieved via the reporter oligonucleotide which can be detected on an in situ platform (e.g., by detecting one or more barcode sequences present in the reporter oligonucleotide or present in a probe that binds to the reporter oligonucleotide) and/or using a spatially barcoded capture probe array and subsequent sequencing of spatially barcoded molecules comprising the reporter oligonucleotide sequence (or a complement thereof). In some embodiments, detection and identification of the antigen of the detection complex is achieved via detection of the reporter oligonucleotide and not a signal associated with the scaffold of the detection complex itself, and the scaffold may but does not need to be or comprise an optically detectable moiety, such as a fluorophore (e.g., a fluorochrome such as PE or FITC or any fluorophore disclosed in Section III-iv).

In some embodiments, a biological sample may be contacted with a detection complex comprising an antigen presenting molecule bound to an antigen peptide specific to the TCR of a target T cell. In this context, the reporter oligonucleotide bound to the detection complex may but does not need to comprise a sequence encoding the antigen peptide or a portion thereof. In some aspects, the reporter oligonucleotide comprises a sequence corresponding to the antigen peptide or a portion thereof. The reporter oligonucleotide may comprise a barcode region for identifying the antigen peptide or portion thereof. In some embodiments, the barcode region comprises one or more barcode sequences that can be detected in order to identify the antigen (e.g., antigen peptide) of the detection complex, thereby identifying the antigen specificity of the cell receptor (e.g., T cell receptor) that is bound to the antigen and identifying the target cell (e.g., peptide-responsive T cell). Each of the one or more barcode sequences in the barcode region may comprise consecutive nucleic acids or nucleic acids that are not consecutive in the sequence of the reporter oligonucleotide. The barcode region may also serve as a code for identifying the detection complex to which the reporter oligonucleotide is attached after amplification (e.g., in situ RCA of a circular or circularizable probe bound to the reporter oligonucleotide) and/or sequencing (e.g., sequencing of a spatially barcoded oligonucleotide comprising a sequence of the reporter oligonucleotide or complement thereof).

In some embodiments, provided herein are a plurality (e.g., a panel) of detection complexes each comprising one or more reporter oligonucleotides. In some embodiments, the plurality of detection complexes collectively comprise a library of reporter oligonucleotides for identifying each detection complex to which one or more of the reporter oligonucleotides are attached, thereby identifying the antigen loaded onto the detection complex. In some embodiments, each detection complex comprises an antigen loaded thereon and a reporter oligonucleotide, and each reporter oligonucleotide comprises a unique barcode region with a distinct nucleotide sequence that allows identification of a particular reporter oligonucleotide among reporter oligonucleotides in the plurality of detection complexes. As such, in some embodiments, a reporter oligonucleotide can be uniquely associated with the identity of the antigen (e.g., an amino acid sequence of a peptide antigen) which is loaded onto the detection complex comprising the reporter oligonucleotide. In some embodiments, the unique barcode region in a reporter oligonucleotide enables monitoring of interactions between the detection complex and the targets to which it binds, e.g., TCRs or antigenic peptide-responsive T cells. The barcode region may vary in length depending on the size of the reporter oligonucleotide. Thus, the barcode region is not limited to any specific length, but may comprise between about 5 and about 100 nucleotides, for instance, between about 6 and about 20 nucleotides, or between about 8 and about 16 nucleotides.

In some embodiments, a reporter oligonucleotide uniquely identifies the antigen loaded on the detection complex comprising the reporter oligonucleotide, and the antigen presenting molecule (e.g., MHC) in each of the plurality of detection complexes may be of the same type (e.g., allotype), such as an MHC molecule of the H-2Kb, HLA-A*02:01, HLA-B*07:02, HLA-A*11:01, or HLA-A*24:02 type. In some embodiments, the antigen presenting molecules (e.g., MHCs) in the plurality of detection complexes may be of different types. For example, as a consequence of MHC restriction, a T cell that responds to a peptide presented by one MHC allotype does not respond to another peptide bound by that same MHC allotype or to the same peptide bound to another MHC allotype. As such, in some embodiments, a reporter oligonucleotide may uniquely identify the antigen/antigen presenting molecule combination in the detection complex comprising the reporter oligonucleotide, from among other antigen/antigen presenting molecule combinations. For example, reporter oligonucleotides 1 to 3 can be used to uniquely identify three different pMHC complexes from one another: peptide 1 loaded on MHC of allotype 1, peptide 2 loaded on MHC of allotype 1, and peptide 1 loaded on MHC of allotype 2, where peptides 1 and 2 are of different sequences and MHC allotypes 1 and 2 are different.

In some embodiments, the barcode region can be amplified. In some embodiments, the barcode region is amplified and detected in situ. For example, the biological sample is contacted with a probe or probe set comprising a sequence complementary to the barcode region of the reporter oligonucleotide. The probe or probe set thus hybridizes with the barcode region or the barcode sequence of the reporter oligonucleotide. In some embodiments, upon hybridization to the reporter oligonucleotide, the probe or probe set is ligatable using the reporter oligonucleotide as template. In some aspects, a splint is used as a template, with or without gap filling prior to ligation. The splint may hybridize to the reporter oligonucleotide. In some aspects, the splint does not hybridize to the reporter oligonucleotide. In some embodiments, the probe or probe set is ligated using the reporter oligonucleotide and/or the splint as template, thereby circularizing the probe or probe set hybridized to the reporter oligonucleotide. Amplification (e.g., rolling circle amplification) and hybridization of a detectable probe to the rolling circle product may enable in situ detection of the reporter oligonucleotide of the detection complex bound (e.g., via the antigen/antigen presenting molecule complex) to a receptor in the sample.

In some embodiments, the reporter oligonucleotide may additionally comprise a capture region, a unique molecular identifier (UMI) region, a primer binding region, and/or an adapter region. The reporter oligonucleotide or a portion thereof may be captured by a capture agent, wherein the capture agent is at a location on a substrate, generating a spatially labeled polynucleotide. The spatially labeled polynucleotide may comprise (i) a sequence of the reporter oligonucleotide or complement thereof and (ii) a sequence of the spatial barcode or complement thereof. This allows for determining a sequence of the spatially labeled polynucleotide to detect the spatial barcode and the reporter oligonucleotide, thereby detecting the corresponding antigen or antigen/antigen presenting molecule combination at the location in the biological sample. Exemplary methods for using spatially barcoded capture agent to capture the reporter oligonucleotide (or a portion or product thereof) include but are not limited to those described in US 2021/0262018, which is incorporated herein by reference.

### C) Scaffolds

In some embodiments, a scaffold disclosed herein includes a molecule or molecular complex that is part of the detection complex and to which is attached one or more antigen presenting molecules (APMs) (e.g., MHC molecules) and/or one or more reporter oligonucleotides. In some embodiments, the antigen-loaded detection complex comprises a scaffold conjugated to one or more MHC molecules. In some aspects, the scaffold is covalently or non-covalently conjugated to the one or more MHC molecules. In some embodiments, the scaffold comprises a protein, a fluorochrome (e.g., PE, FITC, etc.), a polysaccharide, a streptavidin, a avidin, or mutant, variant, or analog thereof. In some aspects, the polysaccharide is a glucan. In some aspects, the polysaccharide comprises dextran.

In some embodiments, the scaffold may be conjugated to the antigen presenting molecules (e.g., MHC molecules) via one or more linkers. A linker may be connected to an antigen presenting molecule (e.g., an MHC molecule) or a complex thereof covalently or non-covalently. The linker may comprise or be linked to an affinity tag that binds to the scaffold. In some embodiments, an affinity tag may be a moiety located on or linked to an antigen presenting molecule (e.g., an MHC molecule). In some embodiments, the scaffold comprises a binding partner of an affinity tag. Examples of affinity tags include, but are not limited to, biotin, antibody epitopes, His-tags, streptavidin, avidin, strep-tactin, polyhistidine, peptides, haptens and metal ion chelates etc. Exemplary pairs of affinity tags and binding partners that may be used include, but are not limited to, biotin/streptavidin, biotin/avidin, biotin/neutravidin, biotin/strep-tactin, poly-His/metal ion chelate, peptide/antibody, glutathione-S-transferase/glutathione, epitope/antibody, maltose binding protein/amylase, and maltose binding protein/maltose. In some embodiments, the scaffold comprises multimerized streptavidin.

In some embodiments, the scaffold can be of a shape and dimension that prevents, reduces, and/or minimizes steric hindrance among two or more molecules directly or indirectly conjugated thereto, including, for example, molecules of a reporter oligonucleotide, molecules of an affinity tag or binding partner, molecules of a linker, molecules of an antigen presenting molecule, and/or molecules of an antigen. In some embodiments, the scaffold can be linear, elongated, or spherical. In some embodiments, the scaffold can be a flexible molecule or complex. In some embodiments, the scaffold can have a molecular weight of at least or about 15 kDa, at least or about 30 kDa, at least or about 60 kDa, at least or about 90 kDa, at least or about 120 kDa, at least or about 150 kDa, at least or about 180 kDa, at least or about 210 kDa, at least or about 240 kDa, at least or about 270 kDa, at least or about 300 kDa, at least or about 330 kDa, at least or about 360 kDa, at least or about 390 kDa, at least or about 420 kDa, at least or about 450 KDa, or greater. In some embodiments, the scaffold can comprise a protein of a molecular weight of any of the aforementioned values or in a range between any two of the aforementioned values. For instance, in some embodiments, the scaffold can be a protein that is between about 150 kDa and about 360 kDa, e.g., about 250 kDa, in molecular weight.

In some embodiments, the scaffold comprises an affinity tag and/or a binding partner thereof. In some embodiments, the scaffold comprises the affinity tag and/or binding partner thereof and a fluorochrome. In some embodiments, the scaffold comprises a streptavidin conjugated to a protein such as a flourochrome. In some embodiments, the detection complex can comprise a flourochrome (e.g., PE) as a scaffold, which scaffold is conjugated to multiple molecules of a streptavidin or avidin or analog or variant thereof, thereby oligomerizing the streptavidin or avidin or analog or variant thereof. In some embodiments, the detection complex can comprise a flourochrome (e.g., PE) conjugated to four streptavidin molecules, thus forming a tetrameric complex. In some embodiments, a reporter oligonucleotide is conjugated to the scaffold (e.g., flourochrome such as PE). In some embodiments, a reporter oligonucleotide is not conjugated to the affinity tag or the binding partner such that the reporter oligonucleotide does not interfere with the interaction between the affinity tag and the binding partner.

In some embodiments, the scaffold is configured to accommodate at least two, four, six, eight, or more molecules of an affinity tag or a binding partner thereof. In some embodiments, the scaffold is a protein (e.g., a fluorochrome such as PE) conjugated with four or more streptavidin molecules and one or more molecules of a reporter oligonucleotide. In some embodiments, the scaffold is optically detectable and/or comprises an optically detectable label. In some embodiments, an optical signal associated with the scaffold may but does not need to be detected in a method disclosed herein, whereas optical signals associated with the reporter oligonucleotides or probes targeting the reporter oligonucleotides (or optical signals associated with a product (e.g., RCA product) of the reporter oligonucleotides or of the probes targeting the reporter oligonucleotides) are detected in order to identify the reporter oligonucleotides and the corresponding antigen or antigen/APM combination.

The detection complex may be provided with more than one antigen-free or antigen peptide-bound MHC molecule (e.g., MHC Class I) in order to ensure sufficient binding of antigens and/or antigen presenting molecules to cells (e.g., antigen peptide-responsive T cells) in the sample. Thus, in some aspects, the detection complex is a multimer. In some embodiments, the detection complex comprises at least two antigen-free or antigen peptide-bound MHC class I molecules, such as at least three antigen-free or antigen peptide-bound MHC class I molecules (trimer), preferably four antigen-free or antigen peptide-bound MHC class I molecules (tetramer). In some embodiments, the detection complex comprises four peptide-bound MHC class I molecules attached to a streptavidin scaffold via non-covalent interactions between streptavidin and a biotin tag on each antigen-free or antigen peptide-bound MHC class I molecule. In some embodiments, the peptide-loaded detection complex comprises four MHC molecules each comprising a biotin or mutant, variant, or analog thereof that binds to one of the four subunits of the tetramer. In some embodiments, each of the four MHC molecules is bound to one molecule of an antigen peptide. The detection complex may comprise at least one reporter oligonucleotide, which may be attached to the scaffold, the antigen-free or antigen peptide-bound MHC class I molecule, or the antigen presenting molecule. In some embodiments, one or more reporter oligonucleotides are attached to the scaffold.

In some embodiments, the peptide-loaded detection complex binds to one, two, or more TCR molecules of the T cell. Generally, T cell receptors have a low avidity and fast off-rates for MHC-peptide complexes. In some embodiments, only one pMHC complex of a tetramer may engage with its cognate TCR. In some embodiments, the tetramer may rotate due to the fast-off rates in a manner wherein at any given time one of the four pMHC complexes of a tetramer is bound to one cognate TCR of a cell. In some embodiments, the ability to rotate enables the tetramer to bind to a TCR with increased avidity and stability. In some embodiments, multiple pMHC molecules of a tetramer (e.g., three out of four pMHC molecules) can simultaneously engage multiple TCRs of a cell.

In some aspects, the avidity of the detection complex to the TCRs can be increased using a scaffold (e.g., dextran) that is linear or linearized. Multiple pMHC molecules, such as ten pMHC molecules, may be simultaneously conjugated to the scaffold. The pMHC molecules may bind the same antigen peptide. In these examples, the reporter oligonucleotide may be directly conjugated to the scaffold.

In a detection complex or an antigen presenting molecule multimer, the scaffold may be non-covalently bound to an affinity tag located on the antigen presenting molecule or MHC molecule. The non-covalent binding may be formed by hydrophobic interactions, hydrophilic interactions, ionic interactions, van der walls forces, hydrogen bonding, and combinations thereof. Thus, a detection complex or an antigen presenting molecule multimer may comprise a scaffold conjugated to more than one affinity tag, such as at least two affinity tags, such as at least 4 affinity tags, such as at least five affinity tags, each located on or linked to an antigen presenting molecule. For instance, a detection complex may comprise a scaffold conjugated to four affinity tags located on or linked to four peptide-MHC molecules. An antigen presenting molecule and an affinity tag may be covalently or non-covalently linked. A non-covalent link may be formed by hydrophobic interactions, hydrophilic interactions, ionic interactions, van der walls forces, hydrogen bonding, and combinations thereof.

### D) Antigen Peptides

The antigen peptides suitable for use herein may essentially come from any source. The antigen source may include, but is not limited to, a human, a virus, a bacterium, a parasite, a plant, a fungus, or a tumor. In some embodiments, the antigenic peptide of the pMHC molecule is derived from a source selected from the group consisting of a human, a virus, a bacterium, a parasite, a plant, a fungus, and a tumor. Antigen peptides used herein can include peptides comprising at least a portion of an antigen selected from the group consisting of tumor associated antigens, tumor specific antigens, neoantigens, autoantigens, infectious agents, toxins, allergens, haptens, or a combination thereof.

In some embodiments, the antigen peptides can include synthetically produced peptides to identify the T cell repertoires present in the biological sample. In some embodiments, the peptides are from libraries of synthetically produced peptides, including, but not limited to, peptide libraries produced by introducing random mutations into various positions of a template peptide. In other embodiments, a peptide library includes up to about 1×10², about 2×10², about 3×10², about 4×10², about 5×10², about 6×10², about 7×10², about 8×10², about 9×10², about 1×10³, about 2×10³, about 3×10³, about 4×10³, about 5×10³, about 6×10³, about 7×10³, about 8×10³, about 9×10³, and about 1×10⁴ member peptides. In some cases, T cell recognition is dominated by only a few amino acids in the core of the peptide, and in these cases, libraries with only a few hundred to a few thousand members can be used to identify functional peptide-MHC complexes for use in the present disclosure.

Candidate MHC-binding peptides of the present disclosure can be generated or obtained by any suitable methods. In certain aspects, the candidate MHC-binding peptides can be peptides produced by hydrolysis. In other embodiments, the candidate MHC-binding peptides are synthetically produced peptides, including randomly generated peptides, specifically designed peptides, and peptides where at least some of the amino acid positions are conserved among several peptides and the remaining positions are random. In the present disclosure, the term “antigen peptide” refers to a peptide that is capable of binding to the binding groove of a major histocompatibility complex (MHC) molecule to form a peptide-MHC (pMHC) complex. The pMHC complex can present the antigen peptide to immune cells to induce for instance, a T-cell receptor dependent immune response. In some embodiments, the antigen presenting molecule (e.g., a non-classical MHC) can present antigens to immune cells such as NK-T cells (e.g., T cell receptors of Natural killer-T cells). In some embodiments, the antigen presenting molecule can present antigens to immune cells such as B cells (e.g., B cell receptors of memory B cells). In some embodiments, the antigen presenting molecule binds to an antigen peptide to form an antigen peptide-loaded detection complex.

In some embodiments, the antigen peptide is between 5 and 40 amino acid residues in length, inclusive. In some embodiments, the antigen peptide length comprises 5, 6, 7, 8, 9, 10, 11, 12, 13, 14, 15, 16, 17, 18, 19, 20, 21, 22, 23, 24, 25, 26, 26, 28, 29, 30, 31, 32, 33, 34, 35, 36, 37, 38, 39, or 40 amino acid residues. In some embodiments, the antigen peptide is between about 7 and about 15 amino acid residues in length, inclusive. In some embodiments, the antigen peptide comprises 7, 8, 9, 10, 11, 12, 13, 14, or 15 amino acid residues. In some embodiments, the antigen peptide is between about 10 and about 25 amino acid residues in length, inclusive (e.g., MHC Class I bound peptides). In some embodiments, the antigen peptide comprises 10, 11, 12, 13, 14, 15, 16, 17, 18, 19, 20, 21, 22, 23, 24, or 25 amino acid residues (e.g., MHC Class II bound peptides).

In some embodiments, the antigen peptide comprises one or more post-translational modifications (PTMs) at one or more amino acid residues. Exemplary PTMs include but are not limited to phosphorylation, methylation (e.g., mono-, di-, or tri-methylation), acylation, acetylation, hydroxylation, deamidation, eliminylation, glycosylation (e.g., N-, O-, or C-linked glycosylation, glypiation, or phosphoglycosylation), AMPylation, ADP-ribosylation, prenylation, ubiquitination (e.g., mono- or poly-ubiquitination), UBL-protein conjugation (e.g., mono- or poly-SUMOylation), arginylation, nitrosylation, lipidation, and/or disulfide bond formation. In some embodiments, one or more PTMs of an antigen peptide can be associated with a disease or condition, e.g., post-translational modifications specific to a tumor cell can include aberrant phosphorylation.

In some embodiments, the MHC-binding peptide is from a library of candidate antigen peptides, wherein the each of the peptides in the library comprises conserved amino acids in a specific sequence sufficient to enable the peptide to bind to the peptide binding groove of an MHC molecule. In some aspects, the MHC-binding peptide is from a library of candidate antigen peptides, wherein each of the peptides in the library comprises between about 4 and 5 conserved amino acids in a specific sequence sufficient to enable the peptide to bind to the peptide binding groove of an MHC molecule. A library of candidate peptides (candidate antigen peptides or MHC-binding peptides) is produced by genetically engineering the library using polymerase chain reaction (PCR) or any other suitable technique to construct a DNA fragment encoding the peptide. With PCR techniques, by using oligonucleotides that are randomly mutated within particular triplet codons, the resultant fragment pool encodes all possible combination of codons at these positions. Certain amino acid positions may be maintained constant, which may be the conserved amino acids that are required for binding to the MHC peptide binding groove and which do not contact the T cell receptor.

Provided herein is a method for analyzing a biological sample, comprising a) contacting the biological sample with a plurality of peptide-loaded detection complexes, wherein each peptide-loaded detection complex comprises a major histocompatibility complex (MHC) multimer and molecules of an antigen peptide bound to the MHC molecules of the multimer, wherein the MHC multimer comprises a scaffold conjugated to a reporter oligonucleotide comprising a barcode region corresponding to the antigen peptide or a portion thereof or the antigen peptide/MHC combination in the peptide-loaded detection complex; b) allowing the plurality of peptide-loaded detection complexes to bind to T cell receptors (TCRs) of T cells at multiple locations in the biological sample; c) fixing and/or crosslinking the biological sample; d) contacting the fixed and/or crosslinked biological sample with a plurality of probes or probe sets that each hybridizes to the barcode region or a portion thereof; and e) detecting a signal associated with the probe or probe set or a product thereof at a particular location in the biological sample, thereby detecting a particular reporter oligonucleotide and the corresponding antigen peptide or antigen peptide/MHC combination at the particular location.

For the purpose of simultaneous high-throughput screening for many different antigen peptides, it is to be understood that a plurality of different peptide-loaded detection complexes are generated and contacted with the sample. In some embodiments, a plurality of peptide-loaded detection complexes are provided, and the sample is contacted with a plurality or library of different peptide-loaded detection complexes.

The antigen peptides presented by the peptide-bound MHC (pMHC) molecules ultimately decides which type of T cells will be identified by the method described herein. The antigen peptide may be an already known immunogenic epitope (e.g., of a virus or a tumor cell), thus enabling detection of the presence of T cells responsive to this antigen and the subsequent diagnosis of a viral infection or cancer. The antigen peptide may also be an unknown epitope, with the detection of T cells responsive to this epitope being indicative for the presence of an immunogenic amino acid sequence within this peptide, thus enabling the identification of immunogenic regions or epitopes in e.g., a polypeptide. In cancer immunotherapy, there is a great interest in understanding T-cell recognition of cancer-associated epitopes as well as mutation-derived neo epitopes and the potential to explore these for therapeutic strategies. In most cases, for the comprehensive evaluation of neoepitope recognition, screening of large pMHC libraries is necessary. The ability to apply peptide-loaded detection complexes will ease the process of generating such large pMHC libraries. In some embodiments, the antigen peptides are selected from cancer-associated epitopes, virus epitopes, self-epitopes and variants thereof. In some embodiments, the antigenic peptides are neoepitopes.

The promiscuous nature of T-cell receptors (TCRs) is fundamental for the ability to recognize a large range of pathogens; however, this feature makes it challenging to understand and control T-cell recognition. Existing technologies provide limited information about the key requirements for T-cell recognition and the ability of TCRs to cross-recognize structurally related elements. The detection complexes described herein serves as a “one-pot” strategy to establish the patterns that govern TCR recognition of pMHC, and at the same time reveal spatial information of these patterns in tissues. Detection complexes as described herein may be used to determine the affinity-based hierarchy of TCR interactions with MEW loaded with peptide variants, and apply this knowledge to understand the recognition motif, herein termed the TCR fingerprint. The peptide-loaded detection complexes are a flexible solution suited for the fast preparation of large libraries of antigenic peptides necessary for high quality TCR fingerprinting. Determination of TCR fingerprints is valuable strategy for understanding T-cell interactions and assessing potential cross-recognition prior to selection of TCRs for clinical development.

An aspect of the present disclosure relates to a method for determining the interaction between a T cell receptor (TCR) or antigenic peptide responsive T cell and a library of antigen peptides, the method comprising the following steps: contacting the biological sample with a plurality of peptide-loaded detection complexes, wherein each peptide-loaded detection complex comprises a major histocompatibility complex (MHC) multimer and molecules of an antigen peptide bound to the MHC molecules of the multimer, wherein the MHC multimer comprises a scaffold conjugated to a reporter oligonucleotide comprising a barcode region corresponding to the antigen peptide or a portion thereof or the antigen peptide/MHC combination in the peptide-loaded detection complex; allowing the plurality of peptide-loaded detection complexes to bind to T cell receptors (TCRs) of T cells at multiple locations in the biological sample; fixing and/or crosslinking the biological sample; contacting the fixed and/or crosslinked biological sample with a plurality of probes or probe sets that each hybridizes to the barcode region or a portion thereof; and detecting a signal associated with the probe or probe set or a product thereof at a particular location in the biological sample, thereby detecting a particular reporter oligonucleotide and the corresponding antigen peptide or antigen peptide/MHC combination at the particular location.

In some embodiments, a method disclosed herein further comprises detecting a TCR transcript encoding a component of the TCR specific to the antigen peptide or antigen peptide/MHC combination at the particular location, wherein the TCR transcript comprises a TCRα V-J join, a TCRβ V-D-J join, a TCRγ V-J join, or a TCRδ V-D-J join. In some embodiments, a signal associated with the TCR transcript is detected at the particular location in the biological sample. In some embodiments, the spatially labeled polynucleotide is sequenced, wherein the spatially labeled polynucleotide comprises (i) a sequence of the TCR transcript or complement thereof and (ii) a sequence of a spatial barcode or complement thereof, and the spatial barcode corresponds to a location on a substrate corresponding to the particular location in the biological sample. In some embodiments, the plurality of peptide-loaded detection complexes comprise antigen peptides or antigen peptide/MHC combinations for binding to TCRs of different antigen specificities. In some embodiments, the biological sample comprises clonal populations of T cells expressing TCRs of different antigen specificities.

In some embodiments, the identity of a particular TCR in the sample can be determined by detecting a TCR transcript corresponding to the particular TCR. In some embodiments, the identity of a particular TCR in the sample can be determined by using a detectable antibody that binds to the particular TCR. In cases where the identity of one or more TCRs are determined, the spatial distribution of each TCR in the sample and its antigen binding specificity can be detected using a method disclosed herein. Thus, in some embodiments, a method disclosed herein is used for obtaining a TCR fingerprint, by revealing not only what peptide antigens are cross-recognized by a particular TCR, but also where the cross-reactive T cells are in a tissue.

In some embodiments, the library or plurality of antigen peptides for use in a method disclosed herein comprises at least 25 different antigen peptides, such as at least 50, such as at least 100, such as at least 500, such as at least 1,000, such as at least 10,000 different antigen peptides. In some embodiments, the library or plurality of antigen peptides comprises at least 100,000 different antigen peptides, such as at least 1,000,000 different antigenic peptides. Given the flexible platform for fast generation of a variety of antigen peptide libraries provided by the methods and detection complexes described herein, it is possible to establish detailed TCR fingerprints against large complex and/or diverse antigen peptide libraries in a simple on demand manner. In some embodiments, the plurality or library of antigen peptides comprises the human peptidome. In some embodiments, the plurality or library of antigen peptides is generated from a target antigen peptide and single position variations of said target antigen peptide. In some embodiments, the plurality or library of antigen peptides is selected from naturally occurring peptides. Antigenic peptides suitable for the method for obtaining a TCR fingerprint include, but is not limited to, antigen peptides known or suspected to be pathogenic. In some embodiments, the target antigen peptide is selected from a cancer-associated epitope, a virus epitope and a self-epitope. In some embodiments, detection complexes described herein are used for determining the interactions between multiple T cell receptors or antigen peptide responsive T cells and a plurality or library of antigen peptides, while also revealing spatial distribution of these interactions in a tissue sample.

### III. In Situ Assays

In some aspects, provided herein are methods comprising in situ assays using an optical readout (e.g., microscopy as a readout) for detection of any of the detection complexes as described in Section II, e.g., nucleic acid detection, hybridization, or other detection or determination methods. In some aspects, detection or determination of a sequence of one, two, three, four, five, or more nucleotides of a target nucleic acid (e.g., reporter oligonucleotide) is performed in situ in a cell in an intact tissue. In some embodiments, the assay comprises detecting the presence or absence of an amplification product (e.g., RCA product). In some embodiments, the present disclosure provides methods for high-throughput profiling of a large number of targets in situ, such as reporter oligonucleotides, e.g., for detecting and/or quantifying T cell repertoires in tissues, organs or organisms.

In some aspects, provided herein is a method comprising analyzing biological targets based on in situ hybridization of probes comprising nucleic acid sequences. In some embodiments, the method comprises sequential hybridization of barcoded probes that directly or indirectly bind to biological targets (e.g., the reporter oligonucleotides described in Section II) in a sample. In some embodiments, a detectably-labelled probe directly binds to one or more reporter oligonucleotides. In some embodiments, a detectably-labelled probe indirectly binds to one or more reporter oligonucleotides, e.g., via one or more bridging nucleic acid molecules.

Exemplary in situ detection methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594 incorporated herein by reference), hybridization chain reaction, assembly of topologically catenated DNA structures using serial rounds of chemical ligation (clampFISH), signal amplification via hairpin-mediated concatemerization (e.g., as described in US 2020/0362398 incorporated herein by reference), e.g., primer exchange reactions such as signal amplification by exchange reaction (SABER) or SABER with DNA-Exchange (Exchange-SABER). In some embodiments, a non-enzymatic signal amplification method may be used.

In some aspects, an in situ hybridization based assay is used to localize and analyze nucleic acid sequences (e.g., a DNA or RNA molecule of a reporter oligonucleotide comprising one or more specific barcode sequences of interest) within a native biological sample, e.g., a portion or section of tissue. In some embodiments, the in situ assay is used to analyze the presence, absence, an amount or level of reporter oligonucleotides (e.g., reporter oligonucleotides in detection complexes bound to TCRs of T cells) in a biological sample, while preserving spatial context. In some embodiments, the present disclosure provides compositions and methods for in situ hybridization using directly or indirectly labeled molecules, e.g., complementary DNA or RNA or modified nucleic acids, as probes that bind or hybridize to a reporter oligonucleotide within a biological sample of interest.

Nucleic acid probes or probe sets, in some examples, may be labelled with radioisotopes, epitopes, hapten, biotin, or fluorophores, to enable detection of the location of specific nucleic acid probes or probe sets in tissues.

In some embodiments, provided herein is a method comprising in situ hybridization of detectable probes to localize and/or measure abundance of the reporter oligonucleotides. In some embodiments, detectable probes are hybridized to pre-determined nucleic acid targets (e.g., reporter oligonucleotides). In some embodiments, the reporter oligonucleotides and/or detectable probes comprises one or more barcodes.

In some embodiments, one or more barcodes of a probe are targeted by detectably labeled detection oligonucleotides, such as fluorescently labeled oligonucleotides. In some embodiments, one or more decoding schemes are used to decode the signals, such as fluorescence, for sequence determination. In some embodiments, the decoding of the barcodes of a probe or probe set comprises one or more decoding rounds of hybridizing and detecting detection oligonucleotides.

In some aspects, circularizable probes (e.g., padlock probes) are used to target reporter oligonucleotides of interest in situ. In some embodiments, the circularizable probes comprise oligonucleotides with ends that are complementary to a target sequence (e.g., barcode region on the reporter oligonucleotides). Upon hybridization of circularizable probes to the target sequence, enzymes may be used to ligate the ends of the circularizable probes, and catalyze the formation of circularized DNA.

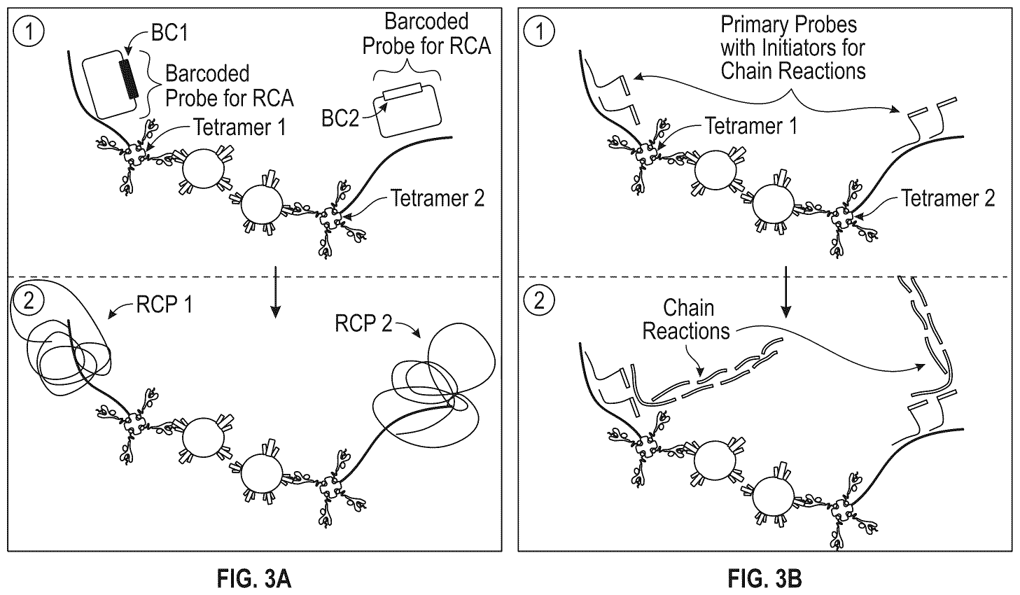

In some aspects, the ends of the circularizable probes are in close proximity upon hybridization to the target reporter oligonucleotides, to allow ligation and circularization of the circularizable probe (FIG. 3A). The circularizable probes may additionally comprise one or more barcode sequences. In alternative aspects, there may be a gap between the ends of the circularizable probes upon hybridization to the target reporter oligonucleotides, that must be filled with nucleic acids (e.g., by DNA polymerization), prior to ligation of the ends of the circularizable probes and circularization. In some aspects, the gap between to ends of the circularizable probes is of variable length, e.g., up to four base pairs, and can allow reading out the reporter oligonucleotides sequence. In some embodiments, upon hybridization to the reporter oligonucleotide, the probe or probe set is ligatable using the reporter oligonucleotide and/or a splint as template, with or without gap filling prior to ligation, optionally wherein the splint hybridizes to the reporter oligonucleotide or the splint does not hybridize to the reporter oligonucleotide. In some embodiments, the DNA polymerase has strand displacement activity. In some embodiments, the DNA polymerase may instead not have strand displacement activity, such as the polymerase used in barcode in situ target sequencing (BaristaSeq) which provides read-length of up to 15 bases using a gap-filling circularizable probe or probe set (e.g., padlock probe) approach. See, e.g., Chen et al., Nucleic Acids Res. 2018, 46, e22, incorporated herein by reference.

A method described herein may comprise DNA circularization and amplification (e.g., rolling circle amplification), at the location of circular or circularizable probes (FIG. 3A). In some embodiments, amplification results in multiple repeats of circular or circularizable probe sequences, including barcode sequences (or complements thereof) in the circular or circularizable probes. Detecting of the barcode sequences (or complements thereof) may be performed using sequencing-by-ligation, sequencing-by-synthesis, or sequential hybridization of detectably labeled probes. In some embodiments, amplicons are stabilized by crossing-linking described herein, during the sequence determination process.

### i) Probes and Hybridization Complexes

In some aspects, the methods disclosed herein involve the use of one or more probes or probe sets that hybridize to a target nucleic acid, such as a reporter oligonucleotide as described in Section II.

In some embodiments, the tissue has previously been processed, e.g., fixed, embedded, frozen, or permeabilized using any of the steps and/or protocols described in Section IX. In some embodiments, the target nucleic acid is a reporter oligonucleotide conjugated to a detection complex bound to a cell receptor (e.g., TCR of a T cell) in a biological sample.

In some embodiments, an additional target nucleic acid such as a TCR transcript encoding a component of the TCR specific to the antigen peptide or antigen peptide/WIC combination may be detected. In some embodiments, the TCR transcript comprises a TCRα V-J join, a TCRβ V-D-J join, a TCRγ V-J join, or a TCRδ V-D-J join. The TCR transcript may be detected simultaneously or after detection of the reporter oligonucleotide.

In some embodiments, the probe or probe set for hybridization to the reporter oligonucleotide of a detection complex disclosed herein is linear, branched, circular, or circularizable. Exemplary probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary. In some embodiments, a primary probe (e.g., a DNA probe that directly binds to a reporter oligonucleotide) is amplified through rolling circle amplification, e.g., using a circular probe or a circularized probe as a template (FIG. 3A). In some embodiments, the primary probes, such as a circularizable probe (e.g., padlock probe), contain one or more barcodes. In some embodiments, one or more barcodes are indicative of a sequence in the target nucleic acid, such as a reporter oligonucleotide (e.g., barcode sequences in the reporter oligonucleotide, that correspond to the antigen peptide or a portion thereof, or the antigen/antigen presenting molecule combination).

In some embodiments, the probe or one or more probe molecules in the probe set may be fluorescent or fluorescently labeled. The probe or probe set comprises a covalently linked fluorophore. The probe or probe set bound to the reporter oligonucleotide further directly or indirectly binds to a fluorescent or fluorescently labeled probe.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation, e.g., for detecting a TCR transcript encoding a component of the TCR. See, e.g., US 2020/0224244 which is hereby incorporated by reference in its entirety. In some embodiments, the probe set is a SNAIL probe set. See, e.g., US 2019/0055594, which is hereby incorporated by reference in its entirety. In some embodiments, provided herein is a probe or probe set capable of proximity ligation, for instance a proximity ligation assay for RNA (e.g., PLAYR) probe set. See, e.g., US 2016/0108458, which is hereby incorporated by reference in its entirety.

In some embodiments, a probe or probe set disclosed herein may comprise a probe comprising a 3′ or 5′ overhang upon hybridization to a nucleic acid molecule (e.g., a reporter oligonucleotide disclosed herein) or a product thereof in the sample; a probe comprising a 3′ overhang and a 5′ overhang upon hybridization to the nucleic acid molecule or product thereof; a circular probe that hybridizes to the nucleic acid molecule or product thereof; a circularizable probe or probe set that hybridizes to the nucleic acid molecule or product thereof; a probe or probe set that hybridizes to the nucleic acid molecule or product thereof and comprises a split hybridization region (e.g., a split barcode region) configured to hybridize to a splint; and a combination thereof. In some embodiments, the 3′ overhang and/or the 5′ overhang of the probe or probe set can each independently comprise one or more barcode sequences.

In some embodiments, a circular probe can be indirectly hybridized to the target nucleic acid (e.g., reporter oligonucleotide). In some embodiments, the circular construct is formed from a probe set capable of proximity ligation, for instance a proximity ligation in situ hybridization (PLISH) probe set. See, e.g., US 2020/0224243 which is hereby incorporated by reference in its entirety.

In some embodiments, a probe disclosed herein comprise 1, 2, 3, 4, 5, 6, 7, 8, 9, 10, 11, 12, 13, 14, 15 or more, 20 or more, 32 or more, 40 or more, or 50 or more barcode sequences. The barcode sequences may be positioned anywhere within the nucleic acid probe. If more than one barcode sequences are present, the barcode sequences may be positioned next to each other, and/or interspersed with other sequences. In some embodiments, two or more of the barcode sequences may also at least partially overlap. In some embodiments, two or more of the barcode sequences in the same probe do not overlap. In some embodiments, all of the barcode sequences in the same probe are separated from one another by at least a phosphodiester bond (e.g., they may be immediately adjacent to each other but do not overlap), such as 1, 2, 3, 4, 5, 6, 7, 8, 9, 10 or more nucleotides apart.

The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

In some embodiments, the number of distinct barcode sequences in a population of nucleic acid probes is less than the number of distinct targets (e.g., nucleic acid analytes and/or protein analytes) of the nucleic acid probes, and yet the distinct targets may still be uniquely identified from one another, e.g., by encoding a probe with a different combination of barcode sequences. However, not all possible combinations of a given set of barcode sequences need be used. For instance, each probe may contain 1, 2, 3, 4, 5, 6, 7, 8, 9, 10, 11, 12, 13, 14, 15, etc. or more barcode sequences. In some embodiments, a population of nucleic acid probes may each contain the same number of barcode sequences, although in other cases, there may be different numbers of barcode sequences present on the various probes. In some embodiments, the barcode sequences or any subset thereof in the population of nucleic acid probes can be independently and/or combinatorially detected and/or decoded.

An exemplary probe set and hybridization complex are shown in FIG. 3A, where a circular or circularizable probe (e.g., padlock probe) directly hybridizes to the reporter oligonucleotide of the detection complex. The circular or circularizable probe comprises a region that hybridizes to a complementary barcode region in the reporter oligonucleotide. The circular or circularizable probe may additionally comprise a probe barcode region (e.g., BC1 or BC2 shown in FIG. 3A) that does not hybridize to the reporter oligonucleotide. The region of the probe that hybridizes to a complementary barcode region and/or the probe barcode region in each circular or circularizable probe can be used to identify the circular or circularizable probe, thereby identifying the reporter oligonucleotide and the corresponding antigen or antigen/APM combination.

The circularizable probe can be ligated using the reporter oligonucleotide and/or a splint as template, thereby circularizing the probe. After probe hybridization and/or any circularization steps to provide a circular probe, in some embodiments the circular probe is amplified, e.g., in a RCA reaction, to generate an amplified molecule wherein the RCA product (RCP) comprises multiple copies of the complements of the barcode sequence of the reporter oligonucleotide and/or probe barcode regions. The RCA can comprise a linear RCA, a branched RCA, a dendritic RCA, or any combination thereof. In some embodiments, after amplification, the method further comprises a detecting step. In some embodiments, the detecting step comprises contacting the RCA product with detectable probes that hybridize to the barcode sequences or complements thereof in sequential hybridization cycles, thereby identifying the reporter oligonucleotide and the corresponding antigen or antigen/APM combination. The detectable probes can be fluorescent or fluorescently labeled (such as a fluorescently labeled detection oligo) that is capable of hybridizing to one or more of the barcode sequences or complementary sequences thereof. In some embodiments, the detectable probes each hybridizes to one or more fluorescent or fluorescently labeled probes.

In some embodiments, upon hybridization to the reporter oligonucleotide, the probe or probe set comprises a region which is an initiator for a hybridization chain reaction (HCR) or which hybridizes to an initiator for HCR (FIG. 3B). In some embodiments, upon hybridization to the reporter oligonucleotide, the probe or probe set comprises a region which is an initiator for linear oligonucleotide hybridization chain reaction (LO-HCR) or which hybridizes to an initiator for LO-HCR. In some embodiments, upon hybridization to the reporter oligonucleotide, the probe or probe set comprises a region which is a primer for primer exchange reaction (PER) or which hybridizes to a primer for PER. In some embodiments, upon hybridization to the reporter oligonucleotide, the probe or probe set comprises a region which is a pre-amplifier for branched DNA (bDNA) or which hybridizes to a pre-amplifier for bDNA.

### ii) Ligation

In some aspects, after formation of a hybridization complex comprising nucleic acid probes and/or probe sets with the reporter oligonucleotides described above, the assay further comprises one or more steps such as ligation, and/or amplification of the probe or probe set hybridized to the target nucleic acid. In some embodiments, the provided methods involve ligating one or more polynucleotides that are part of a hybridization complex that comprises a target nucleic acid (e.g., reporter oligonucleotide) for in situ analysis. In some embodiments, the ligation involves template dependent ligation. In some embodiments, upon hybridization to the reporter oligonucleotide, the probe or probe set is ligatable using the reporter oligonucleotide as a template. In some embodiments, upon hybridization to the reporter oligonucleotide, the probe or probe set is ligatable using a splint as template. In some embodiments, the splint hybridizes to the reporter oligonucleotide (e.g., SNAIL like). In some embodiments, the splint does not hybridize to the reporter oligonucleotide. In some embodiments, the ligation involves template independent ligation. In some embodiments, the ligation involves enzymatic ligation.

In FIG. 3A, the one or more reporter oligonucleotides can aid in the ligation of the circularizable probe. In some embodiments, one or more suitable probes can be used and ligated, wherein the one or more probes comprise a sequence that is complementary to the one or more reporter oligonucleotides (or portion thereof). The probe may comprise one or more barcode sequences. In some embodiments, the one or more reporter oligonucleotide may serve as a primer for rolling circle amplification (RCA) of the circularized probe. In some embodiments, a nucleic acid other than the one or more reporter oligonucleotide is used as a primer for rolling circle amplification (RCA) of the circularized probe. For example, a nucleic acid capable of hybridizing to the circularized probe at a sequence other than sequence(s) hybridizing to the one or more reporter oligonucleotide can be used as the primer for RCA. In other examples, the primer in a SNAIL probe set used as the primer for RCA.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity.

In some embodiments, the ligation herein is a direct ligation. In some embodiments, the ligation occurs with or without gap filling prior to ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, circularizable probe or probe set (e.g., padlock probes), or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tm) of the DNA strands. This selectively reduces the concentration of annealed mismatched substrates (expected to have a slightly lower Tm around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

iii) Amplification

In some embodiments, the methods described herein comprise the step of amplifying one or more polynucleotides, for instance amplification of a sequence in a circular or circularizable probe disclosed herein. In some embodiments, the amplifying is achieved by performing rolling circle amplification (RCA). In other embodiments, a primer that hybridizes to the circular or circularizable probe is added and used as such for amplification.

In some embodiments, a removing step is performed to remove molecules that are not specifically hybridized to the target nucleic acid (e.g., reporter oligonucleotide) and/or the circular probe. In some embodiments, the removing step is performed to remove unligated probes. In some embodiments, the removing step is performed after ligation and prior to amplification.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball containing multiple copies of the cDNA. Any suitable techniques for rolling circle amplification (RCA) may be used such as linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 November 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:e1 18, 2001; Dean et al. Genome Res. 11:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such Phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is Phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N⁶-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix.

Exemplary modification and polymer matrix that can be employed in accordance with the provided embodiments comprise those described in, for example, U.S. Pat. No. 10,494,662, US US 2016/0024555, US 2018/0251833, and US 2021/0215581, all of which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or non-covalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from reporter oligonucleotides corresponding to a pMHC bound to a TCR of a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

### iv) Detection and Analysis

In some aspects, after formation of a hybridization complex comprising reporter oligonucleotide and probe and/or probe sets and further processing (e.g., ligation, amplification or any combination thereof described in Section III), the method further includes detection of the probe or probe set hybridized to the target reporter oligonucleotide or any products generated therefrom or a derivative thereof.

Provided herein is a method for detecting a signal associated with the probe or probe set or a product thereof at the location in the biological sample. In some embodiments, the detecting comprises imaging the biological sample to detect the signal, optionally wherein the imaging comprises fluorescent microscopy. In some embodiments, the detecting does not comprise contacting the biological sample with an antibody that binds to the peptide-loaded detection complex. In some embodiments, the detecting comprises sequential hybridization, sequencing by hybridization, sequencing by ligation, sequencing by synthesis, sequencing by binding, or a combination thereof.

In some aspects, the provided methods involve analyzing, e.g., detecting or determining, one or more sequences present in the polynucleotides (e.g., probe or probe set) and/or in a product or derivative thereof, such as in an amplified circular or circularized probe. In some embodiments, the detection comprises providing detection probes, such as probes for performing a chain reaction that forms an amplification product, e.g., HCR. In some embodiments, the analysis comprises determining the sequence of all or a portion of the amplification product. In some embodiments, the analysis comprises detecting a sequence present in the amplification product. In some embodiments, the sequence of all or a portion of the amplification product is indicative of the identity of a region of interest in a target nucleic acid. In other embodiments, the provided methods involve analyzing, e.g., detecting or determining, one or more sequences present in the polynucleotide probes (e.g., a barcode sequence present in a circular or circularizable probe or product thereof).

For example, FIGS. 3A-3B depict exemplary methods of detecting antigen-loaded detection complexes bound to receptors (e.g., TCRs) in a biological sample in situ. FIG. 3A illustrates an exemplary method comprising the generation of rolling circle amplification products (RCPs) for in situ detection. BC1 and BC2 represent two different barcodes present on the barcoded circular or circularizable probes for RCA. FIG. 3B depicts an exemplary method comprising performing chain reactions and signal amplification for in situ detection.

In some embodiments, a method disclosed herein may also comprise one or more signal amplification components. In some embodiments, the present disclosure relates to the detection of nucleic acids sequences in situ using probe hybridization and generation of amplified signals associated with the probes, wherein background signal is reduced and sensitivity is increased. In some embodiments, a reporter oligonucleotide disclosed herein can be detected in with a method that comprises signal amplification. Exemplary signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594 incorporated herein by reference), hybridization chain reaction, assembly of topologically catenated DNA structures using serial rounds of chemical ligation (clampFISH), signal amplification via hairpin-mediated concatemerization (e.g., as described in US 2020/0362398 incorporated herein by reference), e.g., primer exchange reactions such as signal amplification by exchange reaction (SABER) or SABER with DNA-Exchange (Exchange-SABER). In some embodiments, a non-enzymatic signal amplification method may be used.

The detectable reactive molecules may comprise tyramide, such as used in tyramide signal amplification (TSA) or multiplexed catalyzed reporter deposition (CARD)-FISH. In some embodiments, the detectable reactive molecule may be releasable and/or cleavable from a detectable label such as a fluorophore. In some embodiments, a method disclosed herein comprises multiplexed analysis of a biological sample comprising consecutive cycles of probe hybridization, fluorescence imaging, and signal removal, where the signal removal comprises removing the fluorophore from a fluorophore-labeled reactive molecule (e.g., tyramide). Exemplary detectable reactive reagents and methods are described in U.S. Pat. No. 6,828,109, US 2019/0376956, US 2019/0376956, US 2022/0026433, US 2022/0128565, and US 2021/0222234, all of which are incorporated herein by reference in their entireties.

In some embodiments, hybridization chain reaction (HCR) can be used for in situ signal amplification and detection. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target reporter oligonucleotide. HCR is described in detail in Dirks and Pierce, 2004, PNAS, 101(43), 15275-15278 and in U.S. Pat. Nos. 7,632,641 and 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401, all of which are herein incorporated in their entireties). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived.

In some embodiments, similar to HCR reactions that use hairpin monomers, linear oligo hybridization chain reaction (LO-HCR) can also be used for in situ signal amplification and detection. In some embodiments, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some embodiments, the first species and/or the second species may not comprise a hairpin structure. In some embodiments, the plurality of LO-HCR monomers may not comprise a metastable secondary structure. In some embodiments, the LO-HCR polymer may not comprise a branched structure. In some embodiments, performing the linear oligo hybridization chain reaction comprises contacting the target nucleic acid molecule with the initiator to provide the initiator hybridized to the target nucleic acid molecule. In any of the embodiments herein, the target nucleic acid molecule and/or the analyte can be a reporter oligonucleotide disclosed herein.

In some embodiments, detection of nucleic acids sequences in situ includes an assembly for branched signal amplification. In some embodiments, the assembly complex comprises an amplifier hybridized directly or indirectly (via one or more oligonucleotides) to a reporter oligonucleotide disclosed herein. In some embodiments, the assembly includes one or more amplifiers each including an amplifier repeating sequence. In some aspects, the one or more amplifiers is labeled. Described herein is a method of using the aforementioned assembly, including for example, using the assembly in multiplexed error-robust fluorescent in situ hybridization (MERFISH) applications, with branched DNA amplification for signal readout. In some embodiments, the amplifier repeating sequence is about 5-30 nucleotides, and is repeated N times in the amplifier. In some embodiments, the amplifier repeating sequence is about 20 nucleotides, and is repeated at least two times in the amplifier. In some aspects, the one or more amplifier repeating sequence is labeled. For exemplary branched signal amplification, see e.g., U.S. Pat. Pub. No. US20200399689A1 and Xia et al., Multiplexed Detection of RNA using MERFISH and branched DNA amplification. Scientific Reports (2019), each of which is fully incorporated by reference herein.

In some embodiments, a reporter oligonucleotide disclosed herein can be detected in with a method that comprises signal amplification by performing a primer exchange reaction (PER). In various embodiments, a primer with domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, a plurality of concatemer primers is contacted with a reporter oligonucleotide described herein. In various embodiments, a reporter oligonucleotide disclosed herein may be contacted with a plurality of concatemer primers and a plurality of labeled probes, see e.g., U.S. Pat. Pub. No. US20190106733, which is incorporated herein by reference, for exemplary molecules and PER reaction components.

In some embodiments, the in situ detection herein can comprise sequencing performed in situ by sequencing-by-synthesis (SBS), for instance, for detecting a sequence of a reporter oligonucleotide disclosed herein or a barcode sequence of a probe that hybridizes to the reporter oligonucleotide. In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, U.S. Pat. No. 8,563,477, US 2005/0100900, WO 06/064199, U.S. Pat. No. 8,715,966, US 2012/0270305, US 2013/0260372, and US 2013/0079232, all of which are herein incorporated by reference in their entireties.

In some embodiments, the in situ detection herein can comprise sequential hybridization, e.g., sequencing by hybridization and/or sequential in situ fluorescence hybridization, for instance, for detecting a sequence of a reporter oligonucleotide disclosed herein or a barcode sequence of a probe that hybridizes to the reporter oligonucleotide. Sequential fluorescence hybridization can involve sequential hybridization of detectable probes comprising an oligonucleotide and a detectable label. In some embodiments, a method disclosed herein comprises sequential hybridization of the detectable probes disclosed herein, including detectably labeled probes (e.g., fluorophore conjugated oligonucleotides) and/or probes that are not detectably labeled per se but are capable of binding (e.g., via nucleic acid hybridization) and being detected by detectably labeled probes. Exemplary methods comprising sequential fluorescence hybridization of detectable probes are described in US 2019/0161796, US 2020/0224244, US 2022/0010358, US 2021/0340618, and US 2023/0039899, all of which are incorporated herein by reference.

In some embodiments, the method comprises generating a signal code sequence at one or more locations in the biological sample on a substrate, the signal code sequence comprising signal codes corresponding to the signals (or absence thereof) associated with detectable probes for in situ hybridization that are sequentially hybridized to the biological sample, wherein the signal code sequence corresponds to a reporter oligonucleotide or a barcode region therein (which can be used to identify the antigen or a portion thereof or the antigen/APM combination in a particular antigen-loaded detection complex that binds to a receptor in the sample), thereby detecting the reporter oligonucleotides at multiple locations in the biological sample and identifying the antigens and their receptors at the multiple locations in the biological sample.

In some embodiments, a method disclosed herein comprises generating rolling circle amplification (RCA) products associated with the reporter oligonucleotides (which are conjugated to the antigen-loaded detection complexes) and/or one or more target nucleic acids in the biological sample on a substrate. In some embodiments, the RCA products are detected in situ in the biological sample on the substrate, thereby detecting the reporter oligonucleotides and/or the one or more target nucleic acids. In some embodiments, each of the RCA products comprises multiple complementary copies of a barcode sequence, wherein the barcode sequence is associated with a reporter oligonucleotide or a target nucleic acid in the biological sample and is assigned a signal code sequence. In some embodiments, the method comprises contacting the biological sample with a first detectable probe comprising (i) a recognition sequence complementary to a sequence in the complementary copies of the barcode sequence and (ii) a reporter. In some embodiments, the method comprises detecting a first signal or absence thereof from the reporter of the first detectable probe hybridized to its corresponding sequence of the complementary copies of the barcode sequence in the RCA product, wherein the first signal or absence thereof corresponds to a first signal code in the signal code sequence. In some embodiments, the method comprises contacting the biological sample with a subsequent detectable probe comprising (i) a recognition sequence complementary to a sequence of the complementary copies of the barcode sequence and (ii) a reporter. In some embodiments, the method comprises detecting a subsequent signal or absence thereof from the reporter of the subsequent detectable probe hybridized to its corresponding sequence of the complementary copies of the barcode sequence in the RCA product, wherein the subsequent signal or absence thereof corresponds to a subsequent signal code in the signal code sequence. In some embodiments, the signal code sequence comprising the first signal code and the subsequent signal code is determined at a location in the biological sample, thereby decoding the barcode sequence and identifying the corresponding reporter oligonucleotide (conjugated to the antigen-loaded detection complex) or the target analyte (e.g., an mRNA) at the location in the biological sample. In some embodiments, multiple sequence code sequences can be determined in parallel at locations in the biological sample, and the sequence code sequences can be decoded using a code book comprising sequence code sequences each assigned to one of multiple different reporter oligonucleotides (conjugated to the antigen-loaded detection complexes) or one of multiple different target analytes (e.g., different mRNAs), thereby detecting the antigen binding characteristics as well as target analytes in cells at locations in the biological sample. As such, detection of the target analyte (e.g., a target nucleic acid) in situ in a particular cell in the biological sample can be correlated with the antigen binding characteristics (e.g., antigen binding specificity analyzed through detecting the reporter oligonucleotides conjugated to the antigen-loaded detection complexes) of the cell, since the reporter oligonucleotide detected in situ in the particular cell identifies the antigen or portion thereof or the antigen/APM combination in the antigen-loaded detection complex that binds to the cell.

In some embodiments, the barcode sequence comprises one or more barcode positions each comprising one or more barcode subunits. In some embodiments, a barcode position in the barcode sequence partially overlaps an adjacent barcode position in the barcode sequence. In some embodiments, the first detectable probe and the subsequent detectable probe are in a set of detectable probes each comprising the same recognition sequence and a reporter. In some embodiments, the reporter of each detectable probe in the set comprises a binding site for a reporter probe comprising a detectable moiety. In some embodiments, the reporter probe binding site of the first detectable probe and the reporter probe binding site of the subsequent detectable probe are the same. In some embodiments, the reporter probe binding site of the first detectable probe and the reporter probe binding site of the subsequent detectable probe are different. In some embodiments, the detectable moiety is a fluorophore and the signal code sequence is a fluorophore sequence uniquely assigned to the target analyte. In some embodiments, the detectable probes in the set are contacted with the biological sample sequentially in a pre-determined sequence which corresponds to the signal code sequence assigned to the barcode sequence. In some embodiments, the detectable probes in the set are contacted with the biological sample to determine signal codes in the signal code sequence until sufficient signal codes have been determined to decode the barcode sequence, thereby identifying the target analyte.

In some embodiments, the in situ detection herein can comprise sequencing performed in situ by sequencing-by-ligation (SBL), for instance, for detecting a sequence of a reporter oligonucleotide disclosed herein or a barcode sequence of a probe that hybridizes to the reporter oligonucleotide. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597, all of which are herein incorporated in their entireties.

In some aspects, the provided methods comprise imaging the amplification product (e.g., amplicon) and/or one or more portions of the polynucleotides, for example, via binding of the detection probe and detecting the detectable label. In some embodiments, the detection probe comprises a detectable label that can be measured and quantitated. The terms “label” and “detectable label” comprise a directly or indirectly detectable moiety that is associated with (e.g., conjugated to) a molecule to be detected, e.g., a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a detectable probe containing a detectable label can be used to detect one or more polynucleotide(s), e.g., a sequence of the probes or probe sets, and/or amplification products described herein. In some embodiments, the methods involve incubating the detectable probe containing the detectable label with the sample, washing unbound detectable probe, and detecting the label, e.g., by imaging.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991), all of which are herein incorporated by reference in their entireties. In some embodiments, exemplary techniques and methods methodologies applicable to the provided embodiments comprise those described in, for example, U.S. Pat. Nos. 4,757,141, 5,151,507 and 5,091,519, all of which are herein incorporated by reference in their entireties. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); US (xanthine dyes); and U.S. Pat. No. 5,688,648 (energy transfer dyes), all of which are herein incorporated by reference in their entireties. Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264, all of which are herein incorporated by reference in their entireties. As used herein, the term “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods are known for custom synthesis of nucleotides having other fluorophores (See, Henegariu et al. (2000) Nature Biotechnol. 18:345, both of which are herein incorporated by reference in their entireties).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62, which is herein incorporated by reference in their entireties).

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a polynucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, and 5,192,782, all of which are herein incorporated by reference in their entireties. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some aspects, the provided methods involve analyzing, e.g., detecting or determining, one or more sequences present in the reporter oligonucleotides and/or in a product or derivative thereof, such as in an amplified circular or circularizable probe that hybridizes to a reporter oligonucleotide. In some cases, the analysis is performed on one or more images captured, and may comprise processing the image(s) and/or quantifying signals observed. For example, the analysis may comprise processing information of one or more cell types, one or more types of markers (e.g., TCRs), a number or level of a marker, and/or a number or level of cells detected in a particular region of the sample. In some embodiments, the analysis comprises detecting a sequence e.g., a barcode present in the sample. In some embodiments, the analysis includes quantification of puncta (e.g., if amplification products are detected). In some cases, the analysis includes determining whether particular cells and/or signals are present that correlate with one or more markers from a particular panel. In some embodiments, the obtained information may be compared to a positive and negative control, or to a threshold of a feature to determine if the sample exhibits a certain feature or phenotype. In some cases, the information may comprise signals from a cell, a region of a tissue sample, and/or comprise readouts from multiple detectable labels. In some case, the analysis further includes displaying the information from the analysis or detection step. In some embodiments, software may be used to automate the processing, analysis, and/or display of data.

### IV. Spatial Assays

In some embodiments, after binding of an antigen-loaded detection complex to receptors in a sample, the assay may further comprise one or more steps for transferring the reporter oligonucleotide of the detection complex (or a product or derivative thereof) to an array for spatial assay (e.g., performing NGS sequencing to determine one or more sequences of the oligonucleotides captured on the array). In some embodiments, a spatial assay disclosed herein is performed without the in situ assays. In some embodiments, the spatial assay disclosed herein is performed following an in situ assay (e.g., using fluorescence microscopy as a readout). In some embodiments, a probe or probe set (e.g., complementary to the reporter oligonucleotide) can be ligated and transferred to an array. In some embodiments, a product (e.g., extension product) or derivative of the ligated probes can be transferred to an array.

In one aspect, provided herein are methods, compositions, apparatus, and systems for spatial analysis of a biological sample, for example, a spatial array-based analysis. Non-limiting aspects of spatial analysis methodologies are described in U.S. Pat. Nos. 10,308,982; 9,879,313; 9,868,979; Liu et al., bioRxiv 788992, 2020; U.S. Pat. Nos. 10,774,372; 10,774,374; WO 2018/091676; U.S. Pat. Nos. 10,030,261; 9,593,365; 10,002,316; 9,727,810; 10,640,816; Rodrigues et al., Science 363(6434):1463-1467, 2019; U.S. Pat. No. 11,447,807; Lee et al., Nat. Protoc. 10(3):442-458, 2015; U.S. Pat. Nos. 10,179,932; 11,085,072; 10,138,509; Trejo et al., PLoS ONE 14(2):e0212031, 2019; U.S. Patent Application Publication No. 2018/0245142; Chen et al., Science 348(6233):aaa6090, 2015; Gao et al., BMC Biol. 15:50, 2017; WO 2017/144338; US 2018/0372736; US 2022/0290228; U.S. Pat. No. 11,597,965; WO 2011/094669; U.S. Pat. Nos. 7,709,198; 8,604,182; 8,951,726; 9,783,841; 10,041,949; WO 2016/057552; US 2021/0238665; U.S. Pat. Nos. 10,370,698; 10,724,078; 10,364,457; 10,317,321; US 2021/0395796; US 2020/0239946; U.S. Pat. Nos. 10,059,990; 11,505,819; 11,104,936; and Gupta et al., Nature Biotechnol. 36:1197-1202, 2018, all of which are herein incorporated by reference in their entireties, and can be used herein in any combination. Further non-limiting aspects of spatial analysis methodologies are described herein.

In some embodiments, the spatial assays disclosed herein comprise capturing a targeted analyte (e.g., reporter oligonucleotide). In some embodiments, provided herein is method for analyzing a biological sample comprising contacting the biological sample with an antigen peptide and a detection complex simultaneously or in any order, wherein the detection complex (e.g., as described in Section II) comprises an antigen presenting molecule monomer or multimer that binds to the antigen to form an antigen-loaded detection complex, wherein the antigen-loaded detection complex is conjugated to a reporter oligonucleotide, the reporter oligonucleotide corresponds to the antigen or a portion thereof or the antigen/antigen presenting molecule combination in the antigen-loaded detection complex, and the antigen-loaded detection complex binds to a receptor on and/or in a cell at a location in the biological sample. In some aspects, the biological sample is on the substrate (e.g., a cover slip with sufficient strength comprising the capture array). In some embodiments, the biological sample is on a second substrate. The biological sample may be positioned between the first substrate (e.g., substrate comprising the capture array) and the second substrate (e.g., slide comprising the biological sample) such that the capture agents are allowed to capture the reporter oligonucleotides or derivatives thereof. In some embodiments, the biological sample is processed to release the reporter oligonucleotides or portion thereof. The permeabilization step (e.g., using Proteinase K) allows the reporter oligonucleotides to migrate onto the capture array substrate and be captured by the capture probes. In some aspects, the permeabilization may be combined with lysing.

In some embodiments, a method disclosed herein comprises transferring one or more analytes (e.g., reporter oligonucleotide) from a biological sample to an array of features on a substrate, each of which is associated with a unique spatial location on the array. Each feature may comprise a plurality of capture agents capable of capturing one or more nucleic acid molecules (e.g., reporter oligonucleotide and/or TCR mRNA), and each of the capture agents of the same feature may comprise a spatial barcode corresponding to a unique spatial location of the feature on the array. In some embodiments, the method comprises capturing the reporter oligonucleotide or a portion thereof by a capture agent (e.g., capture probe). In some aspects, the reporter oligonucleotide is not captured by the capture array prior to this capturing step. The capture probe comprises a capture domain that binds to a capture region on the reporter oligonucleotide or a complement thereof. One or more reactions (e.g., extension, and/or ligation) are performed to generate a spatially labeled polynucleotide sequence comprising (i) a sequence of the captured reporter oligonucleotide or a complement thereof, and (ii) a sequence of the spatial barcode (e.g., spatial barcode of the capture probe) or complement thereof. Subsequent analysis of the transferred analytes includes determining the identity of the analytes and the spatial location of each analyte within the sample. The spatially labeled polynucleotide or a portion thereof may be removed from the substrate (e.g., capture array) for sequencing using any suitable nucleic acid sequencing techniques, including next-generation sequencing (NGS). In some embodiments, the sequence of the spatially labeled polynucleotide is determined to detect the spatial barcode and the reporter oligonucleotide. All or part of the sequence of the generated spatially labeled polynucleotide may be determined. The spatial location of each analyte (e.g., reporter oligonucleotide or complement thereof) within the sample is determined based on the feature to which each analyte is bound in the array, and the feature's relative spatial location within the array. In some embodiments, the method disclosed herein comprises identifying the corresponding antigen or antigen/antigen presenting molecule (e.g., pMHC) combination and its location in the biological sample.

In some embodiments, a method disclosed herein comprises associating a spatial barcode with one or more analytes (e.g., reporter oligonucleotides disclosed herein), in one or more cells such as neighboring cells, such that the spatial barcode identifies the one or more analytes, and/or contents of the one or more cells, as associated with a particular spatial location.

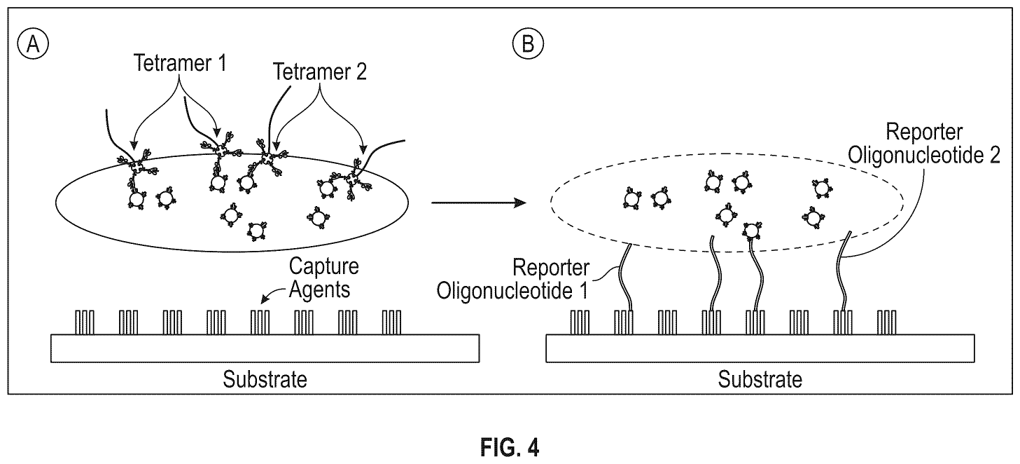

In some embodiments, a method disclosed herein comprises driving target analytes out of a cell and towards a spatially-barcoded array. FIG. 4 depicts an exemplary embodiment, where the biological sample is contacted with a spatially-barcoded array populated with capture probes (FIG. 4A). The TCRs of T cells in the biological are then contacted with detection complexes (e.g., tetramers) conjugated to reporter oligonucleotides as described in Section II. Once the detection complexes bind to the cognate TCRs, the sample is permeabilized (FIG. 4B), allowing the target analyte (e.g., reporter oligonucleotide) to migrate away from the sample and toward the array. The target analyte (e.g., reporter oligonucleotide) interacts with a capture probe on the spatially-barcoded array. Once the target analyte is bound (e.g., hybridizes) to the capture probe, the sample is optionally removed from the array and the capture probes are analyzed in order to obtain spatially-resolved analyte information.

In some embodiments, a method disclosed herein comprises delivering or driving spatially-barcoded nucleic acid molecules (e.g., capture probes) towards and/or into or onto a sample. In some embodiments, a method disclosed herein comprises cleaving spatially-barcoded nucleic acid molecules (e.g., capture probes) from an array and driving the cleaved nucleic acid molecules towards and/or into or onto a sample. Alternatively, the sample may be permeabilized and fixed/crosslinked to restrict mobility of one or more target analytes, while allowing spatially-barcoded capture probes to migrate towards and/or into or onto the sample. Once the spatially-barcoded capture probe is associated with a particular analyte (e.g., reporter oligonucleotides in one or more cells), the sample can be optionally removed for analysis. The sample can be optionally dissociated before analysis. Once the tagged analyte or cell is associated with the spatially-barcoded capture probe, the capture probes can be analyzed to obtain spatially-resolved information about the tagged analyte or cell.

Also disclosed herein are methods for an integrated in situ spatial assay comprising analyzing a first target analyte (e.g., reporter oligonucleotide and products thereof) using in situ analysis (e.g., using fluorescent microscopy or in situ sequencing as a read out) and analyzing a second target analyte (e.g., TCR mRNA) using an array of capture probes (e.g., analyzing TCR transcripts captured on the array using NGS sequencing of spatially barcoded nucleic acid molecules). The in situ analysis of the first analyte (e.g., reporter oligonucleotide and products thereof) may be performed either before, concurrently with, or after analyzing the second target analyte (e.g., TCR mRNA) with the array of capture probes. In some embodiments, the second target nucleic acid (e.g., TCR mRNA) is targeted by one or more nucleic acid probes complementary to the second target nucleic acid (e.g., a cDNA molecule generated from an mRNA molecule). After in situ detection of the first target analyte (e.g., reporter oligonucleotide and amplification products thereof), the sample may be decrosslinked to release the biological molecules (e.g., locked in hydrogel). The rolling circle amplification products may be cleaved. The permeabilization step may facilitates the release of the probes (e.g., TCR mRNA or complements thereof) from the sample to interact with the array of capture probes (e.g., after the in situ analysis). In some embodiments, the reporter oligonucleotide is additionally captured and detected on the capture array. Once the capture probes capture RNA targets (e.g., TCR mRNA transcripts from a sample that has been analyzed in an in situ assay disclosed herein), first strand cDNA created by template switching and reverse transcriptase is then denatured and the second strand is then extended. The second strand cDNA is then denatured from the first strand cDNA, neutralized, and transferred to a tube. cDNA quantification and amplification can be performed using standard techniques discussed herein. The cDNA can then be subjected to library preparation and indexing, including fragmentation, end-repair, A-tailing, and/or indexing PCR steps, followed by an optional library QC step.

In some embodiments, a method disclosed herein comprises detecting one or more other analytes in the biological sample, in addition to detecting a receptor (e.g., TCR) using an antigen-loaded detection complex disclosed herein. In some embodiments, the one or more other analytes comprise a nucleic acid analyte and/or a non-nucleic acid analyte such as a protein. The one or more other analytes can comprise any biomolecule or chemical compound, including a macromolecule such as a protein or peptide, a lipid or a nucleic acid molecule, or a small molecule, including organic or inorganic molecules. The one or more other analytes can be a cell or a microorganism, including a virus, or a fragment or product thereof. The one or more other analytes can be any substance or entity for which a specific binding partner (e.g. an affinity binding partner) can be developed. Such a specific binding partner may be a nucleic acid probe (for a nucleic acid analyte). Alternatively, the specific binding partner may be coupled to a nucleic acid. The one or more other analytes can include nucleic acid molecules, such as DNA (e.g., genomic DNA, mitochondrial DNA, plastid DNA, viral DNA, etc.) and RNA (e.g., mRNA, microRNA, rRNA, snRNA, viral RNA, etc.), and synthetic and/or modified nucleic acid molecules, (e.g., including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The one or more other analytes may include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. The one or more other analytes may include intracellular analytes, such as proteins, protein modifications (e.g., phosphorylation status or other post-translational modifications), nuclear proteins, nuclear membrane proteins, or any combination thereof. The one or more other analytes can be a single molecule or a complex that contains two or more molecular subunits, e.g., including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g., interactions between proteins and nucleic acids, e.g. regulatory factors, such as transcription factors, and DNA or RNA.

Any of the additional analytes described herein, including those exemplified in the preceding paragraph, may be analyzed using an in situ analysis (e.g., using fluorescent microscopy or in situ sequencing as a read out) and/or using an array of capture probes (e.g., using NGS sequencing of spatially barcoded nucleic acid molecules comprising analyte sequences). Optical signals and/or spatially barcoded nucleic acid molecules associated with the receptor (which is targeted by an antigen-loaded detection complex disclosed herein, e.g., a TCR) and those associated with one or more of the additional analytes (e.g., a TCR mRNA of the same or a different TCR, a non-TCR mRNA, a different TCR, or a non-TCR receptor or cellular protein) can be generated, detected, and/or analyzed simultaneously or sequentially in any suitable order.

Exemplary steps for sample preparation, permeabilization, DNA generation (e.g., first strand cDNA generation and second strand generation), DNA amplification (e.g., cDNA amplification) and quality control, and spatial gene expression library construction are disclosed for example in US 2021/0317524, US 2021/0332424. US 2021/0317524, US 2021/0324457. US 2021/0332425, all of which are incorporated herein by reference in their entireties.

### A. Capture Probes

A capture probe herein can comprise any molecule capable of capturing (directly or indirectly) and/or labelling an analyte of interest in a biological sample (e.g., a reporter oligonucleotide). In some embodiments, the capture probe is a nucleic acid. In some embodiments, the capture probe is a conjugate (e.g., an oligonucleotide-antibody conjugate). In some embodiments, the capture probe includes a barcode (e.g., a spatial barcode and/or a unique molecular identifier (UMI)) and a capture domain.

In some embodiments, analytes in a biological sample can be pre-processed prior to interaction with a capture probe. For example, prior to interaction with capture probes, polymerization reactions catalyzed by a polymerase (e.g., DNA polymerase or reverse transcriptase) are performed in the biological sample. In some embodiments, a primer for the polymerization reaction includes a functional group that enhances hybridization with the capture probe. The capture probes can include appropriate capture domains to capture biological analytes of interest (e.g., reporter oligonucleotides and/or TCR transcripts).

In some aspects, a reverse transcriptase (RT) catalyzed reaction may take place during hybridization of one or more nucleic acid probes to a nucleic acid target in a biological sample for an in situ assay. In some embodiments, the RT reaction converts one or more RNA analytes (e.g., TCR mRNA) in the biological sample to DNA for the in situ assay and/or a spatial assay. In some embodiments, the one or more nucleic acid probes comprise a probe that is ligated with another probe or to itself. For example, a circularizable probe can be ligated using RNA-templated and/or DNA-templated ligation.

In some embodiments, a reverse transcriptase (RT) catalyzed reaction takes place after ligation of a nucleic acid probe with another probe or to itself, wherein the nucleic acid probe hybridizes to a nucleic acid target (e.g., TCR mRNA) in a biological sample for an in situ assay. In some embodiments, the RT reaction converts one or more RNA analytes in the biological sample to DNA for the in situ assay and/or a spatial assay.

In some embodiments, biological analytes are pre-processed for library generation via next generation sequencing. For example, analytes can be pre-processed by addition of a modification (e.g., ligation of sequences that allow interaction with capture probes). In some embodiments, analytes are fragmented using fragmentation techniques. Fragmentation can be followed by a modification of the analyte. For example, a modification can be the addition through ligation of an adapter sequence that allows hybridization with the capture probe. In some embodiments, where the analyte of interest is RNA (e.g., TCR mRNA), poly(A) tailing is performed. Addition of a poly(A) tail to RNA that does not contain a poly(A) tail can facilitate hybridization with a capture probe that includes a capture domain with a functional amount of poly(dT) sequence.

In some embodiments, prior to interaction with capture probes, ligation reactions catalyzed by a ligase are performed in the biological sample. In some embodiments, ligation can be performed by chemical ligation. In some embodiments, the ligation can be performed using click chemistry as further below. In some embodiments, the capture domain includes a DNA sequence that has complementarity to a RNA molecule, where the RNA molecule has complementarity to a second DNA sequence, and where the RNA-DNA sequence complementarity is used to ligate the second DNA sequence to the DNA sequence in the capture domain. In these embodiments, direct detection of RNA molecules is possible.

In some embodiments, prior to interaction with capture probes, target-specific reactions are performed in the biological sample. Examples of target specific reactions include, but are not limited to, ligation of target specific adaptors, probes and/or other oligonucleotides, target specific amplification using primers specific to one or more analytes, and target-specific detection using in situ hybridization, and microscopy. In some embodiments, a capture probe includes capture domains targeted to target-specific products (e.g., amplification or ligation).

In some embodiments, the capture probes may comprise one or more cleavable capture probes, wherein the cleaved capture probe can enter into a non-permeabilized cell and bind to target analytes within the sample. The capture probe may contain a cleavage domain, a cell penetrating peptide, a reporter molecule, and a disulfide bond (—S—S—). In some cases, the capture probe may also include a spatial barcode and a capture domain.

i. Capture Domain

In some embodiments, each capture agent (e.g., a capture probe) comprises at least one capture domain, which may comprise an oligonucleotide that binds specifically to a desired analyte. In some embodiments, a capture domain can be used to capture or detect a desired analyte, such as a reporter oligonucleotide.

In some embodiments, the capture domain comprises a functional nucleic acid sequence configured to interact with one or more analytes, such as one or more different types of reporter oligonucleotides. In some embodiments, the functional nucleic acid sequence can include an N-mer sequence (e.g., a random N-mer sequence), which N-mer sequences are configured to interact with a plurality of nucleic acid molecules, including RNA and/or DNA molecules. In some embodiments, the functional sequence can include a poly(T) sequence, which poly(T) sequences are configured to interact with messenger RNA (mRNA) molecules via the poly(A) tail of an mRNA transcript (e.g., TCR mRNA).

Capture probes can include ribonucleotides and/or deoxyribonucleotides as well as synthetic nucleotide residues that are capable of participating in Watson-Crick type or analogous base pair interactions. In some embodiments, the capture domain is capable of priming a reverse transcription reaction to generate cDNA that is complementary to the captured RNA molecules (e.g., TCR mRNA). In some embodiments, the capture domain of the capture probe can prime a DNA extension (polymerase) reaction to generate DNA that is complementary to the captured DNA molecules. In some embodiments, the capture domain can template a ligation reaction between the captured DNA molecules and a surface probe that is directly or indirectly immobilized on the substrate. In some embodiments, the capture domain can be ligated to one strand of the captured DNA molecules. For example, SplintR ligase along with RNA or DNA sequences (e.g., degenerate RNA) can be used to ligate a single-stranded DNA or RNA to the capture domain. In some embodiments, ligases with RNA-templated ligase activity, e.g., SplintR ligase, T4 RNA ligase 2 or KOD ligase, can be used to ligate a single-stranded DNA or RNA to the capture domain. In some embodiments, a capture domain includes a splint oligonucleotide. In some embodiments, a capture domain captures a splint oligonucleotide.

In some embodiments, the capture domain is located at the 3′ end of the capture probe and includes a free 3′ end that can be extended, e.g. by template dependent polymerization, to form an extended capture probe as described herein. In some embodiments, the capture domain includes a nucleotide sequence that is capable of hybridizing to nucleic acid, e.g. reporter oligonucleotide or TCR mRNA, present in the cells of the tissue sample contacted with the array. In some embodiments, the capture domain can be selected or designed to bind selectively or specifically to a target nucleic acid (e.g., reporter oligonucleotide). For example, the capture domain can be selected or designed to capture mRNA (e.g., TCR mRNA) by way of hybridization to the mRNA poly(A) tail. Thus, in some embodiments, the capture domain includes a poly(T) DNA oligonucleotide, e.g., a series of consecutive deoxythymidine residues linked by phosphodiester bonds, which is capable of hybridizing to the poly(A) tail of mRNA. In some embodiments, the capture domain can include nucleotides that are functionally or structurally analogous to a poly(T) tail. For example, a poly(U) oligonucleotide or an oligonucleotide included of deoxythymidine analogues. In some embodiments, the capture domain includes at least 10, 11, 12, 13, 14, 15, 16, 17, 18, 19, or 20 nucleotides. In some embodiments, the capture domain includes at least 25, 30, or 35 nucleotides.

In some embodiments, random sequences, e.g., random hexamers or similar sequences, can be used to form all or a part of the capture domain. For example, random sequences can be used in conjunction with poly(T) (or poly(T) analogue) sequences. Thus, where a capture domain includes a poly(T) (or a “poly(T)-like”) oligonucleotide, it can also include a random oligonucleotide sequence (e.g., “poly(T)-random sequence” probe). This can, for example, be located 5′ or 3′ of the poly(T) sequence, e.g. at the 3′ end of the capture domain. The poly(T)-random sequence probe can facilitate the capture of the mRNA poly(A) tail. In some embodiments, the capture domain can be an entirely random sequence. In some embodiments, degenerate capture domains can be used.

In some embodiments, a pool of two or more capture probes form a mixture, where the capture domain of one or more capture probes includes a poly(T) sequence and the capture domain of one or more capture probes includes random sequences. In some embodiments, a pool of two or more capture probes form a mixture where the capture domain of one or more capture probes includes poly(T)-like sequence and the capture domain of one or more capture probes includes random sequences. In some embodiments, a pool of two or more capture probes form a mixture where the capture domain of one or more capture probes includes a poly(T)-random sequences and the capture domain of one or more capture probes includes random sequences. In some embodiments, probes with degenerate capture domains can be added to any of the preceding combinations listed herein. In some embodiments, probes with degenerate capture domains can be substituted for one of the probes in each of the pairs described herein.

The capture domain can be based on a particular gene sequence or particular motif sequence or common/conserved sequence, that it is designed to capture (e.g., a sequence-specific capture domain). Thus, in some embodiments, the capture domain is capable of binding selectively to a desired sub-type or subset of nucleic acid. In some embodiments, the capture domain is capable of binding selectively to one or more reporter oligonucleotides disclosed herein.

In some embodiments, a capture domain includes an “anchor” or “anchoring sequence”, which is a sequence of nucleotides that is designed to ensure that the capture domain hybridizes to the intended biological analyte. In some embodiments, an anchor sequence includes a sequence of nucleotides, including a 1-mer, 2-mer, 3-mer or longer sequence. In some embodiments, the short sequence is random. For example, a capture domain including a poly(T) sequence can be designed to capture an mRNA. In such embodiments, an anchoring sequence can include a random 3-mer (e.g., GGG) that helps ensure that the poly(T) capture domain hybridizes to an mRNA. In some embodiments, an anchoring sequence can be VN, N, or NN. Alternatively, the sequence can be designed using a specific sequence of nucleotides. In some embodiments, the anchor sequence is at the 3′ end of the capture domain. In some embodiments, the anchor sequence is at the 5′ end of the capture domain.

ii. Cleavage Domain

Each capture probe can optionally include at least one cleavage domain. The cleavage domain represents the portion of the probe that is used to reversibly attach the probe to an array feature, as will be described further below. Further, one or more segments or regions of the capture probe can optionally be released from the array feature by cleavage of the cleavage domain. As an example spatial barcodes and/or universal molecular identifiers (UMIs) can be released by cleavage of the cleavage domain.

In some embodiments, the cleavage domain linking the capture probe to a feature is a disulfide bond. A reducing agent can be added to break the disulfide bonds, resulting in release of the capture probe from the feature. As another example, heating can also result in degradation of the cleavage domain and release of the attached capture probe from the array feature. In some embodiments, laser radiation is used to heat and degrade cleavage domains of capture probes at specific locations. In some embodiments, the cleavage domain is a photo-sensitive chemical bond (e.g., a chemical bond that dissociates when exposed to light such as ultraviolet light).

Other examples of cleavage domains include labile chemical bonds such as, but not limited to, ester linkages (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)).

In some embodiments, the cleavage domain includes a sequence that is recognized by one or more enzymes capable of cleaving a nucleic acid molecule, e.g., capable of breaking the phosphodiester linkage between two or more nucleotides. A bond can be cleavable via other nucleic acid molecule targeting enzymes, such as restriction enzymes (e.g., restriction endonucleases). For example, the cleavage domain can include a restriction endonuclease (restriction enzyme) recognition sequence. Restriction enzymes cut double-stranded or single stranded DNA at specific recognition nucleotide sequences known as restriction sites. In some embodiments, a rare-cutting restriction enzyme, e.g., enzymes with a long recognition site (at least 8 base pairs in length), is used to reduce the possibility of cleaving elsewhere in the capture probe.

In some embodiments, the cleavage domain includes a poly(U) sequence which can be cleaved by a mixture of Uracil DNA glycosylase (UDG) and the DNA glycosylase-lyase Endonuclease VIII, commercially known as the USER™ enzyme. Releasable capture probes can be available for reaction once released. Thus, for example, an activatable capture probe can be activated by releasing the capture probes from a feature.

In some embodiments, where the capture probe is attached indirectly to a substrate, e.g., via a surface probe, the cleavage domain includes one or more mismatch nucleotides, so that the complementary parts of the surface probe and the capture probe are not 100% complementary (for example, the number of mismatched base pairs can one, two, or three base pairs). Such a mismatch is recognized, e.g., by the MutY and T7 endonuclease I enzymes, which results in cleavage of the nucleic acid molecule at the position of the mismatch.

In some embodiments, where the capture probe is attached to a feature indirectly, e.g., via a surface probe, the cleavage domain includes a nickase recognition site or sequence. Nickases are endonucleases which cleave only a single strand of a DNA duplex. Thus, the cleavage domain can include a nickase recognition site close to the 5′ end of the surface probe (and/or the 5′ end of the capture probe) such that cleavage of the surface probe or capture probe destabilizes the duplex between the surface probe and capture probe thereby releasing the capture probe) from the feature.

Nickase enzymes can also be used in some embodiments where the capture probe is attached to the feature directly. For example, the substrate can be contacted with a nucleic acid molecule that hybridizes to the cleavage domain of the capture probe to provide or reconstitute a nickase recognition site, e.g., a cleavage helper probe. Thus, contact with a nickase enzyme will result in cleavage of the cleavage domain thereby releasing the capture probe from the feature. Such cleavage helper probes can also be used to provide or reconstitute cleavage recognition sites for other cleavage enzymes, e.g., restriction enzymes.

Some nickases introduce single-stranded nicks only at particular sites on a DNA molecule, by binding to and recognizing a particular nucleotide recognition sequence. A number of naturally-occurring nickases have been discovered, of which at present the sequence recognition properties have been determined for at least four. Nickases are described in U.S. Pat. No. 6,867,028, which is incorporated herein by reference in its entirety. In general, any suitable nickase can be used to bind to a complementary nickase recognition site of a cleavage domain. Following use, the nickase enzyme can be removed from the assay or inactivated following release of the capture probes to prevent unwanted cleavage of the capture probes.

Examples of suitable capture domains that are not exclusively nucleic-acid based include, but are not limited to, proteins, peptides, aptamers, antigens, antibodies, and molecular analogs that mimic the functionality of any of the capture domains described herein.

In some embodiments, a cleavage domain is absent from the capture probe. Examples of substrates with attached capture probes lacking a cleavage domain are described for example in Macosko et al., (2015) Cell 161, 1202-1214, the entire contents of which are incorporated herein by reference.

In some embodiments, the region of the capture probe corresponding to the cleavage domain can be used for some other function. For example, an additional region for nucleic acid extension or amplification can be included where the cleavage domain would normally be positioned. In such embodiments, the region can supplement the functional domain or even exist as an additional functional domain. In some embodiments, the cleavage domain is present but its use is optional.

iii. Functional Domain

Each capture probe can optionally include at least one functional domain. Each functional domain typically includes a functional nucleotide sequence for a downstream analytical step in the overall analysis procedure.

In some cases, the nucleic acid molecule (e.g., capture probe) can comprise one or more functional sequences. For example, a functional sequence can comprise a sequence for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence for attachment to a sequencing flow cell for Illumina sequencing. In some cases, the functional sequence can comprise a barcode sequence or multiple barcode sequences. In some cases, the functional sequence can comprise a unique molecular identifier (UMI). In some cases, the functional sequence can comprise a primer sequence (e.g., an R1 primer sequence for Illumina sequencing, an R2 primer sequence for Illumina sequencing, etc.). In some cases, a functional sequence can comprise a partial sequence, such as a partial barcode sequence, partial anchoring sequence, partial sequencing primer sequence (e.g., partial R1 sequence, partial R2 sequence, etc.), a partial sequence configured to attach to the flow cell of a sequencer (e.g., partial P5 sequence, partial P7 sequence, etc.), or a partial sequence of any other type of sequence described elsewhere herein. A partial sequence may contain a contiguous or continuous portion or segment, but not all, of a full sequence, for example. In some cases, a downstream procedure may extend the partial sequence, or derivative thereof, to achieve a full sequence of the partial sequence, or derivative thereof. Examples of such capture probes and uses thereof are described in U.S. Patent Publication Nos. 2014/0378345 and 2015/0376609, the entire contents of each of which are incorporated herein by reference. The functional domains can be selected for compatibility with a variety of different sequencing systems, e.g., 454 Sequencing, Ion Torrent Proton or PGM, Illumina X10, etc., or other platforms from Illumina, BGI, Qiagen, Thermo-Fisher, PacBio, and Roche, and the requirements thereof.

iv. Spatial Barcode

As discussed above, the capture probe can include one or more spatial barcodes (e.g., two or more, three or more, four or more, five or more) spatial barcodes. A “spatial barcode” is a contiguous nucleic acid segment or two or more non-contiguous nucleic acid segments that function as a label or identifier that conveys or is capable of conveying spatial information. In some embodiments, a capture probe includes a spatial barcode that possesses a spatial aspect, where the barcode is associated with a particular location within an array or a particular location on a substrate. Exemplary spatial barcodes are described in U.S. Pat. No. 10,030,261, which is incorporated herein by reference.

A spatial barcode can be part of an analyte (e.g., part of the reporter oligonucleotide), or independent from an analyte (e.g., part of the capture probe). A spatial barcode can be a tag attached to an analyte (e.g., reporter oligonucleotide). A spatial barcode can be unique. In some embodiments where the spatial barcode is unique, the spatial barcode functions both as a spatial barcode and as a unique molecular identifier (UMI), associated with one particular capture probe.

Spatial barcodes can have a variety of different formats. For example, spatial barcodes can include polynucleotide spatial barcodes; random nucleic acid and/or amino acid sequences; and synthetic nucleic acid and/or amino acid sequences. In some embodiments, a spatial barcode is attached to an analyte in a reversible or irreversible manner. In some embodiments, a spatial barcode allows for identification and/or quantification of individual sequencing-reads. In some embodiments, a spatial barcode is a used as a barcode for which fluorescently labeled oligonucleotide probes hybridize to the spatial barcode.

In some embodiments, the spatial barcode is a nucleic acid sequence that does not substantially hybridize to analyte reporter oligonucleotide molecules or TCR mRNAs in a biological sample. In some embodiments, the spatial barcode has less than 80% sequence identity (e.g., less than 70%, 60%, 50%, or less than 40% sequence identity) to the reporter oligonucleotide or TCR mRNAs across a substantial part (e.g., 80% or more) of the nucleic acid molecules in the biological sample.

The spatial barcode sequences can include from about 6 to about 20 or more nucleotides within the sequence of the capture probes. In some embodiments, the length of a spatial barcode sequence can be about 6, 7, 8, 9, 10, 11, 12, 13, 14, 15, 16, 17, 18, 19, 20 nucleotides or longer. In some embodiments, the length of a spatial barcode sequence can be at least about 6, 7, 8, 9, 10, 11, 12, 13, 14, 15, 16, 17, 18, 19, 20 nucleotides or longer. In some embodiments, the length of a spatial barcode sequence is at most about 6, 7, 8, 9, 10, 11, 12, 13, 14, 15, 16, 17, 18, 19, 20 nucleotides or shorter. These nucleotides can be completely contiguous, e.g., in a single stretch of adjacent nucleotides, or they can be separated into two or more separate subsequences that are separated by 1 or more nucleotides. Separated spatial barcode subsequences can be from about 4 to about 16 nucleotides in length. In some embodiments, the spatial barcode subsequence can be about 4, 5, 6, 7, 8, 9, 10, 11, 12, 13, 14, 15, 16 nucleotides or longer. In some embodiments, the spatial barcode subsequence can be at least about 4, 5, 6, 7, 8, 9, 10, 11, 12, 13, 14, 15, 16 nucleotides or longer. In some embodiments, the spatial barcode subsequence can be at most about 4, 5, 6, 7, 8, 9, 11, 12, 13, 14, 15, 16 nucleotides or shorter.

For multiple capture probes that are attached to a common array feature, the one or more spatial barcode sequences of the multiple capture probes can include sequences that are the same for all capture probes coupled to the feature, and/or sequences that are different across all capture probes coupled to the feature. In some embodiments, a plurality of capture probes attached to a common array feature may possess the same spatial barcode, but have different capture domains designed to associate the spatial barcode of the feature with more than one target analyte. For example, a feature may be coupled to two, three, four, five, six, seven, eight, nine, ten, or more different types of spatially-barcoded capture probes, each type of spatially-barcoded capture probe possessing the spatial barcode. In some aspects, capture-probe barcoded constructs can be tailored for analyses of any given analyte associated with a nucleic acid and capable of binding with such a construct. In some embodiments, the analyte is a reporter oligonucleotide analyte capable of binding with a spatially-barcoded capture probe disclosed herein.

Capture probes attached to a single array feature can include identical (or common) spatial barcode sequences, different spatial barcode sequences, or a combination of both. Capture probes attached to a feature can include multiple sets of capture probes. Capture probes of a given set can include identical spatial barcode sequences. The identical spatial barcode sequences can be different from spatial barcode sequences of capture probes of another set.

The plurality of capture probes can include spatial barcode sequences that are associated with specific locations on a spatial array. For example, a first plurality of capture probes can be associated with a first region, based on a spatial barcode sequence common to the capture probes within the first region, and a second plurality of capture probes can be associated with a second region, based on a spatial barcode sequence common to the capture probes within the second region. The second region may or may not be associated with the first region. Additional pluralities of capture probes can be associated with spatial barcode sequences common to the capture probes within other regions. In some embodiments, the spatial barcode sequences can be the same across a plurality of capture probe molecules.

In some embodiments, multiple different spatial barcodes are incorporated into a single arrayed capture probe. For example, a mixed but known set of spatial barcode sequences can provide a stronger address or attribution of the spatial barcodes to a given spot or location, by providing duplicate or independent confirmation of the identity of the location. In some embodiments, the multiple spatial barcodes represent increasing specificity of the location of the particular array point.

v. Unique Molecular Identifier

The capture probe can include one or more (e.g., two or more, three or more, four or more, five or more) Unique Molecular Identifiers (UMIs). A unique molecular identifier is a contiguous nucleic acid segment or two or more non-contiguous nucleic acid segments that function as a label or identifier for a particular analyte, or for a capture probe that binds a particular analyte (e.g., via the capture domain).

A UMI can be unique. A UMI can include one or more specific polynucleotides sequences, one or more random nucleic acid and/or amino acid sequences, and/or one or more synthetic nucleic acid and/or amino acid sequences.

In some embodiments, the UMI is a nucleic acid sequence that does not substantially hybridize to analyte nucleic acid molecules in a biological sample. In some embodiments, the UMI has less than 80% sequence identity (e.g., less than 70%, 60%, 50%, or less than 40% sequence identity) to the nucleic acid sequences across a substantial part (e.g., 80% or more) of the nucleic acid molecules in the biological sample.

The UMI can include from about 6 to about 20 or more nucleotides within the sequence of the capture probes. In some embodiments, the length of a UMI sequence can be about 6, 7, 8, 9, 10, 11, 12, 13, 14, 15, 16, 17, 18, 19, 20 nucleotides or longer. In some embodiments, the length of a UMI sequence can be at least about 6, 7, 8, 9, 10, 11, 12, 13, 14, 15, 16, 17, 18, 19, 20 nucleotides or longer. In some embodiments, the length of a UMI sequence is at most about 6, 7, 8, 9, 10, 11, 12, 13, 14, 15, 16, 17, 18, 19, 20 nucleotides or shorter. These nucleotides can be completely contiguous, e.g., in a single stretch of adjacent nucleotides, or they can be separated into two or more separate subsequences that are separated by 1 or more nucleotides. Separated UMI subsequences can be from about 4 to about 16 nucleotides in length. In some embodiments, the UMI subsequence can be about 4, 5, 6, 7, 8, 9, 10, 11, 12, 13, 14, 15, 16 nucleotides or longer. In some embodiments, the UMI subsequence can be at least about 4, 5, 6, 7, 8, 9, 10, 11, 12, 13, 14, 15, 16 nucleotides or longer. In some embodiments, the UMI subsequence can be at most about 4, 5, 6, 7, 8, 9, 10, 11, 12, 13, 14, 15, 16 nucleotides or shorter.

In some embodiments, a UMI is attached to an analyte in a reversible or irreversible manner. In some embodiments, a UMI allows for identification and/or quantification of individual sequencing-reads. In some embodiments, a UMI is a used as a fluorescent barcode for which fluorescently labeled oligonucleotide probes hybridize to the UMI.

vi. Other Aspects of Capture Probes

For capture probes that are attached to an array feature, an individual array feature can include one or more capture probes. In some embodiments, an individual array feature includes hundreds or thousands of capture probes. In some embodiments, the capture probes are associated with a particular individual feature, where the individual feature contains a capture probe including a spatial barcode unique to a defined region or location on the array.

In some embodiments, a particular feature can contain capture probes including more than one spatial barcode (e.g., one capture probe at a particular feature can include a spatial barcode that is different than the spatial barcode included in another capture probe at the same particular feature, while both capture probes include a second, common spatial barcode), where each spatial barcode corresponds to a particular defined region or location on the array. For example, multiple spatial barcode sequences associated with one particular feature on an array can provide a stronger address or attribution to a given location by providing duplicate or independent confirmation of the location. In some embodiments, the multiple spatial barcodes represent increasing specificity of the location of the particular array point. In a non-limiting example, a particular array point can be coded with two different spatial barcodes, where each spatial barcode identifies a particular defined region within the array, and an array point possessing both spatial barcodes identifies the sub-region where two defined regions overlap, e.g., such as the overlapping portion of a Venn diagram.

In another non-limiting example, a particular array point can be coded with three different spatial barcodes, where the first spatial barcode identifies a first region within the array, the second spatial barcode identifies a second region, where the second region is a subregion entirely within the first region, and the third spatial barcode identifies a third region, where the third region is a subregion entirely within the first and second subregions.

In some embodiments, capture probes attached to array features are released from the array features for sequencing. Alternatively, in some embodiments, capture probes remain attached to the array features, and the probes are sequenced while remaining attached to the array features. Further aspects of the sequencing of capture probes are described in subsequent sections of this disclosure.

In some embodiments, an array feature can include different types of capture probes attached to the feature. For example, the array feature can include a first type of capture probe with a capture domain designed to bind to one type of analyte, and a second type of capture probe with a capture domain designed to bind to a second type of analyte. In general, array features can include one or more (e.g., two or more, three or more, four or more, five or more, six or more, eight or more, ten or more, 12 or more, 15 or more, 20 or more, 30 or more, 50 or more) different types of capture probes attached to a single array feature.

In some embodiments, the capture probe is nucleic acid. In some embodiments, the capture probe is attached to the array feature via its 5′ end. In some embodiments, the capture probe includes from the 5′ to 3′ end: one or more barcodes (e.g., a spatial barcode and/or a UMI) and one or more capture domains. In some embodiments, the capture probe includes from the 5′ to 3′ end: one barcode (e.g., a spatial barcode or a UMI) and one capture domain. In some embodiments, the capture probe includes from the 5′ to 3′ end: a cleavage domain, a functional domain, one or more barcodes (e.g., a spatial barcode and/or a UMI), and a capture domain. In some embodiments, the capture probe includes from the 5′ to 3′ end: a cleavage domain, a functional domain, one or more barcodes (e.g., a spatial barcode and/or a UMI), a second functional domain, and a capture domain. In some embodiments, the capture probe includes from the 5′ to 3′ end: a cleavage domain, a functional domain, a spatial barcode, a UMI, and a capture domain. In some embodiments, the capture probe does not include a spatial barcode. In some embodiments, the capture probe does not include a UMI. In some embodiments, the capture probe includes a sequence for initiating a sequencing reaction.

In some embodiments, the capture probe is immobilized on a feature via its 3′ end. In some instances, the capture probe comprises: an adapter sequence—a barcode (e.g., a spatial barcode)—an optional unique molecular identifier (UMI) sequence—a capture domain. In some embodiments, the capture probe includes from the 3′ to 5′ end: one or more barcodes (e.g., a spatial barcode and/or a UMI) and one or more capture domains. In some embodiments, the capture probe includes from the 3′ to 5′ end: one barcode (e.g., a spatial barcode or a UMI) and one capture domain. In some embodiments, the capture probe includes from the 3′ to 5′ end: a cleavage domain, a functional domain, one or more barcodes (e.g., a spatial barcode and/or a UMI), and a capture domain. In some embodiments, the capture probe includes from the 3′ to 5′ end: a cleavage domain, a functional domain, a spatial barcode, a UMI, and a capture domain.

In some embodiments, a capture probe includes an in situ synthesized oligonucleotide. In some embodiments, the in situ synthesized oligonucleotide includes one or more constant sequences, one or more of which serves as a priming sequence (e.g., a primer for amplifying target nucleic acids). In some embodiments, a constant sequence is a cleavable sequence. In some embodiments, the in situ synthesized oligonucleotide includes a barcode sequence, e.g., a variable barcode sequence. In some embodiments, the in situ synthesized oligonucleotide is attached to a feature of an array.

In some embodiments, a capture probe is a product of two or more oligonucleotide sequences, e.g., two or more oligonucleotide sequences that are ligated together. In some embodiments, one of the oligonucleotide sequences is an in situ synthesized oligonucleotide.

In some embodiments, the capture probe includes a splint oligonucleotide. Two or more oligonucleotides can be ligated together using a splint oligonucleotide and any variety of suitable ligases described herein (e.g., SplintR ligase).

In some embodiments, one of the oligonucleotides includes: a constant sequence (e.g., a sequence complementary to a portion of a splint oligonucleotide), a degenerate sequence, and a capture domain (e.g., as described herein). In some embodiments, the capture probe is generated by having an enzyme add polynucleotides at the end of an oligonucleotide sequence. The capture probe can include a degenerate sequence, which can function as a unique molecular identifier.

A capture probe can include a degenerate sequence, which is a sequence in which some positions of a nucleotide sequence contain a number of possible bases. A degenerate sequence can be a degenerate nucleotide sequence including about or at least 5, 6, 7, 8, 9, 10, 11, 12, 13, 14, 15, 16, 17, 18, 19, 20, 25, 30, 35, 40, 45, or 50 nucleotides. In some embodiments, a nucleotide sequence contains 1, 2, 3, 4, 5, 6, 7, 8, 9, 0, 10, 15, 20, 25, or more degenerate positions within the nucleotide sequence. In some embodiments, the degenerate sequence is used as a UMI.

In some embodiments, a capture probe includes a restriction endonuclease recognition sequence or a sequence of nucleotides cleavable by specific enzyme activities. For example, uracil sequences can be cleaved by specific enzyme activity. As another example, other modified bases (e.g., modified by methylation) can be recognized and cleaved by specific endonucleases. The capture probes can be subjected to an enzymatic cleavage, which removes the blocking domain and any of the additional nucleotides that are added to the 3′ end of the capture probe during the modification process. The removal of the blocking domain reveals and/or restores the free 3′ end of the capture domain of the capture probe. In some embodiments, additional nucleotides can be removed to reveal and/or restore the 3′ end of the capture domain of the capture probe.

In some embodiments, a blocking domain can be incorporated into the capture probe when it is synthesized, or after its synthesis. The terminal nucleotide of the capture domain is a reversible terminator nucleotide (e.g., 3′-O-blocked reversible terminator and 3′-unblocked reversible terminator), and can be included in the capture probe during or after probe synthesis.

vii. Extended Capture Probes

An “extended capture probe” is a capture probe with an enlarged nucleic acid sequence. For example, where the capture probe includes nucleic acid, an “extended 3′ end” indicates that further nucleotides were added to the most 3′ nucleotide of the capture probe to extend the length of the capture probe, for example, by standard polymerization reactions utilized to extend nucleic acid molecules including templated polymerization catalyzed by a polymerase (e.g., a DNA polymerase or reverse transcriptase).

In some embodiments, extending the capture probe includes generating cDNA from the captured (hybridized) RNA. This process involves synthesis of a complementary strand of the hybridized nucleic acid, e.g., generating cDNA based on the captured RNA template (the RNA hybridized to the capture domain of the capture probe). Thus, in an initial step of extending the capture probe, e.g., the cDNA generation, the captured (hybridized) nucleic acid, e.g., RNA, acts as a template for the extension, e.g., reverse transcription, step.

In some embodiments, the capture probe is extended using reverse transcription. For example, reverse transcription includes synthesizing cDNA (complementary or copy DNA) from RNA, e.g., (messenger RNA), using a reverse transcriptase. In some embodiments, reverse transcription is performed while the tissue is still in place, generating an analyte library, where the analyte library includes the spatial barcodes from the adjacent capture probes. In some embodiments, the capture probe is extended using one or more DNA polymerases.

In some embodiments, the capture domain of the capture probe includes a primer for producing the complementary strand of the nucleic acid hybridized to the capture probe, e.g., a primer for DNA polymerase and/or reverse transcription. The nucleic acid, e.g., DNA and/or cDNA, molecules generated by the extension reaction incorporate the sequence of the capture probe. The extension of the capture probe, e.g., a DNA polymerase and/or reverse transcription reaction, can be performed using a variety of suitable enzymes and protocols.

For example, the reporter oligonucleotides from the detection complexes bound to a biological sample (or derivatives thereof comprising a sequence of the reporter oligonucleotide or complement thereof) can be captured onto the capture array slide. In some embodiments, capture probes with capture domains can bind to capture regions on the reporter oligonucleotides. In some embodiments, one or more reactions (e.g., extension, and/or ligation) are performed to generate a spatially labeled polynucleotide sequence comprising a sequence of the reporter oligonucleotide or complement thereof and a sequence of the spatial barcode or complement thereof.

In some embodiments, a full-length DNA molecule is generated. In some embodiments, a “full-length” DNA molecule refers to the whole of the captured nucleic acid molecule (e.g., a reporter oligonucleotide disclosed herein). However, if the nucleic acid, e.g., RNA, was partially degraded in the tissue sample, then the captured nucleic acid molecules will not be the same length as the initial RNA in the tissue sample. In some embodiments, the 3′ end of the extended probes, e.g., first strand cDNA molecules, is modified. For example, a linker or adaptor can be ligated to the 3′ end of the extended probes. This can be achieved using single stranded ligation enzymes such as T4 RNA ligase or Circligase™ (available from Epicentre Biotechnologies, Madison, WI). In some embodiments, template switching oligonucleotides are used to extend cDNA in order to generate a full-length cDNA (or as close to a full-length cDNA as possible). In some embodiments, a second strand synthesis helper probe (a partially double stranded DNA molecule capable of hybridizing to the 3′ end of the extended capture probe), can be ligated to the 3′ end of the extended probe, e.g., first strand cDNA, molecule using a double stranded ligation enzyme such as T4 DNA ligase. Any suitable enzymes appropriate for the ligation step may be used and include, e.g., Tth DNA ligase, Taq DNA ligase, Thermococcus sp. (strain 9° N) DNA ligase (9° N™ DNA ligase, New England Biolabs), Ampligase™ (available from Epicentre Biotechnologies, Madison, WI), and SplintR (available from New England Biolabs, Ipswich, MA). In some embodiments, a polynucleotide tail, e.g., a poly(A) tail, is incorporated at the 3′ end of the extended probe molecules. In some embodiments, the polynucleotide tail is incorporated using a terminal transferase active enzyme.

In some embodiments, double-stranded extended capture probes are treated to remove any unextended capture probes prior to amplification and/or analysis, e.g. sequence analysis. This can be achieved by a variety of methods, e.g., using an enzyme to degrade the unextended probes, such as an exonuclease enzyme, or purification columns.

In some embodiments, extended capture probes are amplified to yield quantities that are sufficient for analysis, e.g., via DNA sequencing. In some embodiments, the first strand of the extended capture probes (e.g., DNA and/or cDNA molecules) acts as a template for the amplification reaction (e.g., a polymerase chain reaction).

In some embodiments, the amplification reaction incorporates an affinity group onto the extended capture probe (e.g., RNA-cDNA hybrid) using a primer including the affinity group. In some embodiments, the primer includes an affinity group and the extended capture probes includes the affinity group. The affinity group can correspond to any of the affinity groups described previously.

In some embodiments, the extended capture probes including the affinity group can be coupled to an array feature specific for the affinity group. In some embodiments, the array feature includes avidin or streptavidin and the affinity group includes biotin. In some embodiments, the array feature includes maltose and the affinity group includes maltose-binding protein. In some embodiments, the array feature includes maltose-binding protein and the affinity group includes maltose. In some embodiments, amplifying the extended capture probes can function to release the extended probes from the array feature, insofar as copies of the extended probes are not attached to the array feature.

In some embodiments, the extended capture probe or complement or amplicon thereof is released from an array feature. The step of releasing the extended capture probe or complement or amplicon thereof from an array feature can be achieved in a number of ways. In some embodiments, an extended capture probe or a complement thereof is released from the feature by nucleic acid cleavage and/or by denaturation (e.g., by heating to denature a double-stranded molecule).

### B. Analysis of Captured Analytes

A wide variety of different sequencing methods can be used to analyze spatially barcoded analyte constructs (e.g., described in Section IV.A.vii.). In general, sequenced polynucleotides can be, for example, nucleic acid molecules such as deoxyribonucleic acid (DNA) or ribonucleic acid (RNA), including variants or derivatives thereof (e.g., single stranded DNA or DNA/RNA hybrids, and nucleic acid molecules with a nucleotide analog).

Sequencing of polynucleotides can be performed by various commercial systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification.

Other examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods. Additional examples of sequencing methods that can be used include targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and any combinations thereof.

In some embodiments, direct sequencing of one or more captured analytes is performed by sequencing-by-synthesis (SBS). In some embodiments, a sequencing primer is complementary to a sequence in one or more of the domains of a capture probe (e.g., functional domain). In such embodiments, sequencing-by-synthesis can include reverse transcription and/or amplification in order to generate a template sequence (e.g., functional domain) from which a primer sequence can bind.

SBS can involve hybridizing an appropriate primer, sometimes referred to as a sequencing primer, with the nucleic acid template to be sequenced, extending the primer, and detecting the nucleotides used to extend the primer. Preferably, the nucleic acid used to extend the primer is detected before a further nucleotide is added to the growing nucleic acid chain, thus allowing base-by-base nucleic acid sequencing. The detection of incorporated nucleotides is facilitated by including one or more labelled nucleotides in the primer extension reaction. To allow the hybridization of an appropriate sequencing primer to the nucleic acid template to be sequenced, the nucleic acid template should normally be in a single stranded form. If the nucleic acid templates making up the nucleic acid spots are present in a double stranded form these can be processed to provide single stranded nucleic acid templates using any suitable methods, for example by denaturation, cleavage etc. The sequencing primers which are hybridized to the nucleic acid template and used for primer extension are preferably short oligonucleotides, for example, 15 to 25 nucleotides in length. The sequencing primers can be provided in solution or in an immobilized form. Once the sequencing primer has been annealed to the nucleic acid template to be sequenced by subjecting the nucleic acid template and sequencing primer to appropriate conditions, primer extension is carried out, for example using a nucleic acid polymerase and a supply of nucleotides, at least some of which are provided in a labelled form, and conditions suitable for primer extension if a suitable nucleotide is provided.

Any suitable sequencing methods can be used to analyze barcoded analyte constructs disclosed herein.

### VII. Compositions, Kits, and Systems

In some embodiments, disclosed herein is a composition that comprises a detection complex containing a reporter oligonucleotide and an antigen presenting molecule bound to antigens, e.g., any of the antigen-loaded detection complexes described herein. In some embodiments, the complex further comprises one or more barcode regions on the reporter oligonucleotide.

In some embodiments, disclosed herein is a composition comprising a peptide loaded tetramer that may engage with cognate TCR molecules in a biological sample.

In some embodiments, disclosed herein is a composition that comprises an amplification product containing monomeric units of a barcode sequence complementary to a barcode sequence of a reporter oligonucleotide. In some embodiments, the amplification product is formed using any of the target nucleic acids, probes (e.g., circular or circularizable probes) and any of the amplification techniques described herein.

Also provided herein are kits, for example comprising one or more polynucleotides and reagents for performing the methods provided herein, for example reagents required for one or more steps comprising hybridization, ligation, amplification, detection, sequencing, and/or sample preparation as described herein.

In some embodiments, the kit comprises one or more loadable detection complexes. For instance, the detection complex can comprise an antigen presenting molecule (APM) monomer or multimer capable of binding to an antigen to form an antigen-APM complex, wherein the detection complex is conjugated to a reporter oligonucleotide, and the reporter oligonucleotide corresponds to the antigen or a portion thereof or the antigen/APM combination in the antigen-APM complex. In some embodiments, the kit comprises a plurality of loadable detection complexes to which a customized panel of antigens may be loaded. In some examples, each loadable detection complex can be separately contacted with an antigen to provide an antigen-loaded detection complex, and a plurality of antigen-loaded detection complexes can be combined to contact a sample. In the plurality of detection complexes, each detection complex can: i) comprise a multimer of an antigen presenting molecule (APM) capable of binding to an antigen to form an antigen-APM complex, and ii) be conjugated to a reporter oligonucleotide corresponding to the antigen or a portion thereof or the antigen-APM complex. The APMs in the loadable plurality of detection complexes can be the same or different.

In some embodiments, the kit comprises i) an antigen; ii) a detection complex comprising antigen presenting molecule monomer or multimer capable of binding to the antigen to form an antigen-loaded detection complex, wherein the peptide and/or the detection complex is conjugated to a reporter oligonucleotide, the reporter oligonucleotide corresponds to the antigen peptide or a portion thereof or the antigen/antigen presenting molecule combination in the antigen-loaded detection complex, and the antigen-loaded detection complex is capable of binding to a cell receptor; iii) a probe or probe set capable of hybridizing to the reporter oligonucleotide.

In some embodiments, the kit comprises i) a plurality of peptide-loaded detection complexes, wherein each antigen-loaded detection complex comprises an antigen presenting molecule multimer and molecules of an antigen bound to the antigen presenting molecules of the multimer, wherein the antigen presenting molecule multimer comprises a scaffold conjugated to a reporter oligonucleotide comprising a barcode region corresponding to the antigen peptide or a portion thereof or the antigen/antigen presenting molecule combination in the antigen-loaded detection complex, and the plurality of antigen-loaded detection complexes comprise antigen/antigen presenting molecules capable of binding to cell receptors of different antigen specificities; and ii) a plurality of probes or probe sets each capable of hybridizing to the barcode region or a portion thereof in a particular reporter oligonucleotide of the plurality of antigen-loaded detection complexes. In some embodiments, the plurality of probes or probe sets are circular or circularizable upon hybridization to the reporter oligonucleotides of the plurality of antigen-loaded detection complexes.

In some embodiments, disclosed herein is a kit for analyzing a biological sample, comprising a) contacting the biological sample with an antigen peptide and a detection complex simultaneously or in any order, wherein the detection complex comprises an antigen presenting molecule monomer or multimer that binds to the antigen to form an antigen-loaded detection complex, wherein the antigen-loaded detection complex is conjugated to a reporter oligonucleotide, the reporter oligonucleotide corresponds to the antigen or a portion thereof or the antigen/antigen presenting molecule combination in the antigen-loaded detection complex, and the antigen-loaded detection complex binds to a receptor on and/or in a cell at a location in the biological sample; b) capturing the reporter oligonucleotide or a portion thereof by a capture agent, where in the capture agent is at a location on a substrate and comprises i) a capture domain that binds to the capture region, and ii) a spatial barcode corresponding to the location of the capture agent on the substrate and the corresponding location in the biological sample; c) generating a spatially labeled polynucleotide comprising (i) a sequence of the reporter oligonucleotide or complement thereof and (ii) a sequence of the spatial barcode or complement thereof and d) determining a sequence of the spatially labeled polynucleotide to detect the spatial barcode and the reporter oligonucleotide, thereby detecting the corresponding antigen or antigen/antigen presenting molecule combination at the location in the biological sample.

In some aspects, disclosed herein is a kit for detecting a TCR transcript encoding a component of the TCR specific to the antigen peptide or antigen peptide/WIC combination at the particular location. The TCR transcript comprises a TCRα V-J join, a TCRβ V-D-J join, a TCRγ V-J join, or a TCRδ V-D-J join.

In any of the embodiments herein, the antigen (e.g., antigen peptide) in a particular antigen-loaded detection complex can be selected from the group consisting of tumor associated antigens, tumor specific antigens, neoantigens, autoantigens, infectious agents, toxins, allergens, haptens, or a combination thereof. The antigens in two or more antigen-loaded detection complexes can be selected independently of one another.

In some aspects, disclosed herein is a kit for detecting a signal associated with the TCR transcript at a particular location in the biological sample.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, modified nucleotides, reagents for additional assays.

### VIII. Applications

In some aspects, the provided embodiments can be applied in an in situ method of analyzing clonal populations of T cells comprising T cell receptors (TCRs) of different antigen specificities. For example, peptide-loaded tetramers may be used for in situ detection of the locations of known T cell populations for example in an intact tumor tissue comprising infiltrating immune cells, in which the spatial information has been preserved.

In some aspects, the provided embodiments can be used to identify or detect regions of interest in target nucleic acids. In some aspects, the embodiments can be applied in an imaging or detection method for multiplexed analysis of TCR transcripts (e.g., V-D-J joins).

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug target screening and discovery. In clinical diagnostics, applications comprise, but are not limited to, identifying and pairing of pMHC with cognate TCR sequences of T cells in various tissues such as disease, immune responses for patient samples. In some aspects, the embodiments can be applied to the development of anti-disease vaccinations. In some aspects, the embodiments can be applied to development of TCR therapeutic treatment modalities.

In some aspects, the embodiments can be applied to visualize the spatial distribution of TCR transcripts in whole tissue at cellular or subcellular resolution, indicative of a predisposition towards disease or good health, likelihood of responsiveness to therapy, or in personalized medicine.

### IX. Samples and Sample Processing

A sample disclosed herein can be derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject.

The sample may not be limited to any specific source, but may be peripheral blood mononuclear cells, tumors, tissue, bone marrow, biopsies, serum, blood, plasma, saliva, lymph fluid, pleura fluid, cerebrospinal and synovial fluid. The sample may be extracted from a subject. Samples extracted from individuals may be subjected to the methods described herein to identify and evaluate immune responses during cancer and disease or subsequent to immunotherapy. In the present context, the term “sample” refers to any solution or solid fraction that comprises a population of immune cells (e.g., T cells). The T cell population may contain multiple clones of T cells with different antigen specificities.

In some embodiments, a biological sample disclosed herein can be or comprise a tissue sample comprising a solid tissue, such as brain, liver, lung, kidney, prostate, ovary, spleen, lymph node (including tonsil), thyroid, pancreas, heart, skeletal muscle, intestine, larynx, esophagus, or stomach. In some embodiments, a biological sample disclosed herein can be or comprise a thymus sample, a lymph node tissue sample, a lymphoid tissue sample, such as a gut associated lymphoid tissue sample or a mucosa associated lymphoid tissue sample, a spleen tissue sample, a bone marrow sample, peripheral blood mononuclear cells, a tissue biopsy, and/or a tumor sample. In some embodiments, a biological sample disclosed herein can be or comprise a section of a tissue sample (e.g., an FFPE tissue block), a cell pellet, or a cell block, and the section contains or is suspected of containing immune cells, e.g., T cells and/or B cells. In some embodiments, a biological sample disclosed herein can be or comprise cells or tissues infected with a virus or other intracellular pathogen.

In some embodiments, a biological sample disclosed herein can be or comprise immune cells (e.g., T cells) from cell lines (e.g., T cell lines). In some embodiments, a biological sample disclosed herein can be or comprise immune cells (e.g., T cells) from autologous or allogeneic sources. In some embodiments, a biological sample disclosed herein can be or comprise immune cells (e.g., T cells) from a single individual or multiple individuals, for example, a population of individuals who all suffer or suspected of suffering from a condition or disease. In some embodiments, the condition or disease can comprise a cancer and/or an immune condition or disease, such as inflammation, infection, or an infectious disease. In some embodiments, a biological sample disclosed herein can be or comprise cells from the circulating blood of an individual. In some embodiments, a biological sample disclosed herein can be or comprise cells obtained by apheresis or leukapheresis.

In addition to the subjects described above, a biological sample can be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample is not fixed prior to contacting the biological sample with peptide-loaded detection complexes (e.g., peptide-loaded tetramers). In some embodiments, fixing is performed after the sample is contacted with the peptide-loaded detection complexes and prior to contacting the sample with a probe or probe set for hybridization. In some embodiments, the biological sample can be prepared using formalin-fixation, which are established methods. In some embodiments, cell suspensions can be prepared using formalin-fixation.

A biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or circularizable probe (e.g., padlock probe). In some embodiments, one or more post-fixing step is performed after a hybridization complex comprising a probe and a target (e.g., reporter oligonucleotide) is formed in a sample. In some embodiments, one or more post-fixing step is performed prior to a ligation reaction disclosed herein, such as the ligation to circularize a circularizable probe or probe set (e.g., padlock probe).

A post-fixing step may be performed using any suitable fixation reagent disclosed herein, for example, 3% (w/v) paraformaldehyde in DEPC-PBS.

### (iv) Staining and Immunohistochemistry (IHC)

To facilitate visualization, biological samples (e.g., serial sections of tissues) can be stained using a wide variety of stains and staining techniques. In some aspects, one or more serial sections are stained while the other sections are used for in situ and/or spatial arrays. In some aspects, the staining and in situ and/or spatial arrays are performed using the same tissue section. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be used, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (v) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

### (vi) Crosslinking and De-Crosslinking

In some embodiments, the biological sample is reversibly cross-linked prior to or during an in situ assay. In some embodiments, the biological sample is not crosslinked prior to contacting the sample with detection complexes. In some embodiments, the biological sample is contacted with a first matrix-forming material prior to contacting the sample with detection complexes. In some embodiments, the method comprises forming a first polymerized matrix from the first matrix-forming material, thereby embedding the biological sample in the first polymerized matrix. In some embodiments, the first matrix-forming material does not react with biological molecules in the biological sample. In some embodiments, protein molecules in the biological sample are not crosslinked by the first matrix-forming material or to the first polymerized matrix, In some aspects, the TCRs in the biological sample embedded in the first polymerized matrix remains capable of binding to the WIC monomer or multimer of the peptide-loaded detection complex. In some embodiments, the first polymerized matrix is a hydrogel. The first matrix-forming material may comprise polyethylene glycol (PEG) or a derivative or analog thereof comprising a functional group for crosslinking. In some aspects, the functional group is a click functional group.

In some embodiments, the method further comprises washing the biological sample embedded in the first polymerized matrix. In some embodiments, the method further comprises fixing and/or crosslinking the biological sample after contacting the sample with the detection complexes but prior to contacting the sample with probes for hybridization. In some embodiments, the peptide-loaded detection complex and the TCR bound thereto are fixed and/or crosslinked.

In some embodiments, the biological sample is contacted with a second matrix-forming material after contacting the sample with detection complexes but prior to contacting the sample with probe or probe sets for hybridization. In some embodiments, the sample is contacted with a second matrix-forming material after fixing the biological sample. In some embodiments, the method comprises forming a second polymerized matrix (e.g., hydrogel) from the second matrix-forming material, thereby embedding the biological sample in the second polymerized matrix. The second matrix-forming material crosslinks the peptide-loaded detection complex and the TCR bound thereto to each other, to other molecules in the biological sample, and/or to the second polymerized matrix.

In some aspects, the analytes, polynucleotides and/or amplification product (e.g., amplicon) of an analyte or a probe bound thereto can be anchored to a polymer matrix. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation methods. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(vii) Tissue Permeabilization and Treatment

In some embodiments, a biological sample can be permeabilized to facilitate transfer of analytes out of the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by any suitable non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

(viii) Selective Enrichment of RNA Species

In some embodiments, where RNA is the analyte, one or more RNA analyte species of interest can be selectively enriched. For example, one or more species of RNA of interest (e.g., TCR mRNA) can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some aspects, when two or more analytes are analyzed, a first and second probe that is specific for (e.g., specifically hybridizes to) each RNA or cDNA analyte are used. For example, in some embodiments of the methods provided herein, templated ligation is used to detect gene expression in a biological sample. Probes may be hybridized to the oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

In some embodiments, an oligonucleotide with sequence complementarity to the complementary strand of captured RNA (e.g., cDNA) can bind to the cDNA. For example, biotinylated oligonucleotides with sequence complementary to one or more cDNA of interest binds to the cDNA and can be selected using biotinylation-strepavidin affinity using any of a variety of methods known to the field (e.g., streptavidin beads).

A biological sample may comprise one or a plurality of analytes of interest. Methods for performing multiplexed assays to analyze two or more different analytes (e.g., reporter oligonucleotide and TCR mRNA/V-D-J join) in a single biological sample are provided.

### X. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.”

A “hybridization complex” as used herein may comprise one, two, or more strands or separate molecules. A hybridization complex that comprises three or more strands or separate molecules does not necessarily comprise direct hybridization between every possible pairwise combination thereof, so long as at least two molecules or strands are directly hybridized to each other, or are in the process of binding to or unbinding from each other, at a given time.

“Hybridization conditions” typically include salt concentrations of approximately less than 1 M, often less than about 500 mM and may be less than about 200 mM. A “hybridization buffer” includes a buffered salt solution such as 5% SSPE, or any suitable buffers. Hybridization temperatures can be as low as 5° C., but are typically greater than 22° C., and more typically greater than about 30° C., and typically in excess of 37° C. Hybridizations are often performed under stringent conditions, e.g., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. The melting temperature Tₘ can be the temperature at which a population of double-stranded nucleic acid molecules becomes half dissociated into single strands. Any suitable equation for calculating the Tₘ of nucleic acids may be used. As indicated by standard references, a simple estimate of the Tₘ value may be calculated by the equation, Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeg™ technology by Illumina, Inc., San Diego, Calif; HeliScope™ by Helicos Biosciences Corporation, Cambridge, Ma.; and PacBio RS by Pacific Biosciences of California, Inc., Menlo Park, Calif), sequencing by ion detection technologies (such as Ion Torrent™ technology, Life Technologies, Carlsbad, Calif); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods.

“Multiplexing” or “multiplex assay” herein may refer to an assay or other analytical method in which the presence and/or amount of multiple targets, e.g., multiple nucleic acid target sequences, can be assayed simultaneously by using more than one capture probe conjugate, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

The term “adjacent” as used herein includes but is not limited to being directly linked by a phosphodiester bond. For example, “adjacent” nucleotides or regions on a nucleic acid such as a probe may be separated by a number of nucleotides. For instance, a toehold region and an interrogatory region adjacent to each other may be separated by 0, 1, 2, 3, 4, 5, 6, 7, 8, 9, 10 or more nucleotides in a probe.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

The term “each,” when used in reference to a collection of items, is intended to identify an individual item in the collection but does not necessarily refer to every item in the collection, unless expressly stated otherwise, or unless the context of the usage clearly indicates otherwise.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: Design of Antigen Peptide-Loaded Tetramers

This Example describes the design of an antigen peptide-loaded tetramer (antigen peptide-loaded detection complex). A detection complex (e.g., tetramer) comprises one or more antigen presenting molecule(s) (e.g., MHC Class I or MHC Class II) that may be bound to an antigen peptide to form an antigen peptide-loaded detection complex(es). An antigen presenting molecule (APM) of a detection complex may not be bound to an antigen peptide(s) (e.g., peptide-free detection complex or loadable detection complex). The antigen-loaded detection complex additionally comprises a reporter oligonucleotide that corresponds to the antigen or a portion thereof or the antigen/antigen presenting molecule combination. The reporter oligonucleotide comprises a barcode region for identifying the antigen peptide or portion thereof. The barcode region may comprise one or more barcode sequences and optionally may comprise a capture region, unique molecular identifier (UMI) region, a primer binding region, and/or an adapter region for detection via a spatial capture array The reporter oligonucleotide is covalently or non-covalently bound to a scaffold molecule (e.g., a protein such as a PE fluorochrome). The scaffold molecule is covalently or non-covalently bound to one or more affinity tags coupled to linkers. The linkers are bound to the antigen presenting molecules.

FIG. 1 depicts an antigen peptide-loaded detection complex tetramer comprising a scaffold (e.g., PE fluorochrome conjugated with streptavidin) bound to four peptide bound MHC class I molecules (pMHC Class I) and a reporter oligonucleotide. The reporter oligonucleotide corresponds to the antigen or the antigen/pMHC-Class I combination. Antigen peptide-loaded tetramers can be a powerful tool for screening T cell populations to identify pMHC-responsive T cells. A library of tetramers loaded with a plurality of antigen peptides and corresponding reporter oligonucleotides is applied onto a tissue section (e.g., tumor section with infiltrated immune cells) to detect the pMHC-TCR pairing in situ. The tetramer libraries can also be used for spatial capture array applications to enable sequencing and identification of transcripts of TCRs repertoires present in the tissue samples.

### Example 2: Hybridization, Amplification and In Situ Detection

A library of exemplary antigen peptide-loaded detection complexes (e.g., tetramers) as described in Example 1 above are contacted with T cells in a biological sample, (e.g., a fresh tissue section or a frozen tissue sample comprising tumor infiltrating immune cells), and detected in situ in an exemplary sample.

The library of exemplary antigen peptide-loaded tetramers are allowed to bind to TCRs of T cell populations at multiple locations in the biological sample (FIG. 2). Prior to contacting with the antigen peptide-loaded tetramers, the fresh tissue sample may be contacted with a first matrix-forming material (e.g., hydrogel) to form a first polymerized matrix. The polymerized matrix does not react with the biological molecules (e.g., TCRs of T cells). The TCRs in biological sample are embedded in the polymerized matrix such that the TCRs are locked in place but remain capable of rotating and sufficiently binding to the pMHC multimer of the peptide-loaded detection complex. In some embodiments, protein molecules in the biological sample are not crosslinked by the first matrix-forming material or to the first polymerized matrix. In some embodiments, the first polymerized matrix is a hydrogel, optionally wherein the first matrix-forming material comprises polyethylene glycol (PEG) or a derivative or analog thereof comprising a functional group for crosslinking, optionally wherein the functional group is a click functional group.

Once the library of antigen peptide-loaded tetramers is allowed to bind to TCRs, the sample is washed to remove excess unbound or non-specifically bound tetramers from the tissue sample. The sample is then contacted with a fixative (e.g., paraformaldehyde) to fix the antigen peptide-loaded tetramers and bound TCR. The tissue sample may be mildly crosslinked with a second matrix-forming material post fixation to form a second polymerized matrix. The second matrix-forming material (e.g., hydrogel) crosslinks the antigen peptide-loaded detection complex and the bound TCRs to each other, to other molecules in the tissue sample and to the second polymerized matrix.

A plurality of different probes or probe sets, such as circular or circularizable probes, targeting different target reporter oligonucleotides, are pooled together. The probe or probe set mixture is then contacted with the fixed and crosslinked tissue sample and incubated with hybridization buffer for hybridization to target reporter oligonucleotides. Barcode regions on the probe or probe sets (e.g., BC1, as shown in FIG. 3A (1)) hybridize to complementary barcode regions on the reporter oligonucleotides. The probe or probe sets additionally comprise probe barcode regions (e.g., BC2 as shown in FIG. 3A (1)) that are used as templates for hybridization of additional probes. Alternatively, the probe or probe sets may be fluorescent or fluorescently labeled to enable in situ detection of the bound probes to the reporter oligonucleotide. The probe or probe sets are then ligated to form circular probes or circularized probes. Next, the circular probes or circularized probes are amplified via techniques such as rolling circle amplification (FIG. 3A (2)) using the probe barcode regions for instance, as templates. The rolling circle amplification products (RCPs) are detected via in situ sequencing or linear decoding. Alternatively, the probe or probe sets bound to the reporter oligonucleotides may comprise initiators for chain reactions (e.g., hybridization chain reaction (HCR)) or binding sites for amplifiers for branched DNA amplification (as shown in FIG. 3B (1) and (2)). In both embodiments, combinatorial or sequential decoding can be applied to detect the barcode sequences to determine the identity of the reporter oligonucleotide and thus the tetramer at a given location in the sample. For example, a plurality of cycles of hybridizing, detecting and then removing fluorescently labeled probes enable in situ detection of the bound probes to the reporter oligonucleotide or generated amplification products associated with each reporter oligonucleotide.

### Example 3: Spatial Array-Based Analysis of Peptide-Loaded Tetramers Bound to TCRs

This Example illustrates a method of analyzing a biological sample by contacting with an antigen peptide-loaded detection complex (e.g., tetramer) using spatial array-based analysis (e.g., using NGS sequencing of reporter oligonucleotides captured on the array). The method disclosed herein may be combined with the method disclosed in Example 2, wherein the barcode regions of the reporter oligonucleotide are detected using in situ detection (e.g., using fluorescence microscopy as a readout) followed by the spatial array-based analysis on the same sample.

The biological sample (e.g., fresh tissue or frozen tissue sample) is placed on a thin array slide, such as a cover slip with sufficient strength (FIG. 4 (1)). As shown in FIG. 4 (1) the tissues are then contacted with a library of antigen peptide-loaded tetramers and allowed to bind to TCRs of T cells. Prior to contacting with the antigen peptide-loaded tetramers, the fresh tissue sample is contacted with a first matrix-forming material (e.g., hydrogel) to form a first polymerized matrix. The polymerized matrix does not react with the biological molecules (e.g., TCRs of T cells). The TCRs in biological sample are embedded in the polymerized matric such that the TCRs are locked in place but remain capable of rotating and sufficiently binding to the pMHC multimer of the peptide-loaded detection complex. In some embodiments, the first polymerized matrix is a hydrogel, optionally wherein the first matrix-forming material comprises polyethylene glycol (PEG) or a derivative or analog thereof comprising a functional group for crosslinking, optionally wherein the functional group is a click functional group. Once the library of antigen peptide-loaded tetramers is allowed to bind to TCRs, the sample is washed to remove excess unbound or non-specifically bound tetramers from the tissue sample. The sample is then contacted with a fixative (e.g., paraformaldehyde) to fix the antigen peptide-loaded tetramers and bound TCR. The tissue sample is mildly crosslinked with a second matrix-forming material post fixation to form a second polymerized matrix. The second matrix-forming material (e.g., hydrogel) crosslinks the antigen peptide-loaded detection complex and the bound TCRs to each other, to other molecules in the tissue sample and to the second polymerized matrix.

The tissue sample is then decrosslinked (e.g., using a de-crosslinking catalyst to capture oligonucleotides onto the slides, or to revert the crosslinking if a reversible crosslinker was used) such that the oligonucleotides, and other mRNAs, cDNAs, ligation or amplification products are no longer locked in place (e.g., to a hydrogel). The samples are then permeabilized (e.g., using Proteinase K) or lysed to release the reporter oligonucleotides from the tetramers onto the capture array slide. The capture array comprises immobilized capture probes with capture domains that bind to capture regions on the reporter oligonucleotides. The capture probes also comprise spatial barcodes that correspond to the location of the capture probe on the substrate and the corresponding location of the reporter oligonucleotide (and the TCR) in the biological sample. Next, one or more reactions (e.g., extension, and/or ligation) are performed to generate a spatially labeled polynucleotide sequence comprising a sequence of the reporter oligonucleotide or complement thereof and a sequence of the spatial barcode or complement thereof. The spatially labeled polynucleotide is then removed from the capture array slide and sequenced (e.g., using NGS). All or part of the sequence of the generated spatially labeled polynucleotide is determined and used to detect to detect the spatial barcode and the reporter oligonucleotide (e.g., barcodes within the reporter oligonucleotides that correspond to the antigen/MHC combination), thereby identifying the reporter oligonucleotide that corresponds to the antigen or a portion thereof or the antigen/antigen presenting molecule combination and its location in the biological sample.

As described above, the method disclosed herein may be combined with the method disclosed in Example 2, wherein the barcode regions of the reporter oligonucleotide is detected using in situ detection (e.g., using fluorescence microscopy as a readout as described in Example 2) followed by the spatial array-based analysis of the same sample. The spatial array-based analysis includes reverse transcription of TCR mRNAs (e.g., V-D-J genes) to cDNAs and capture of the TCR transcripts onto the capture array. One or more reactions (e.g., extension, and/or ligation) are performed to generate a spatially labeled polynucleotide sequence comprising a sequence of the TCR transcript or complement thereof and a sequence of a spatial barcode or complement thereof. The spatial barcode corresponds to the location on a substrate corresponding to the particular location of the TCR of a T cell bound to the tetramer.

In this manner, exemplary antigen peptide-loaded tetramers can be used to detect the location of known antigen-responsive T cells in tumor tissues. The method disclosed herein can be applied in high multiplex to discover reactive T cells and their TCR transcripts at locations in tissue sample. This method provides a fast and reliable platform for screening of pMHC-TCR interactions with greater specificities and at high-throughput. Applications of the methods disclosed herein include research and diagnosis and potential drug target discovery. Identification and pairing of pMHC with cognate TCR sequences of T cells in various tissues can be used for development of vaccinations and TCR therapeutic modalities.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1. A method for analyzing a biological sample, comprising:
a) contacting the biological sample with an antigen and a detection complex simultaneously or in any order, wherein:
the detection complex comprises an antigen-presenting molecule (APM) monomer or multimer that binds to the antigen to form an antigen-loaded detection complex, wherein the antigen-loaded detection complex is conjugated to a reporter oligonucleotide,
the reporter oligonucleotide corresponds to the antigen or a portion thereof or the antigen/APM combination in the antigen-loaded detection complex, and
the antigen-loaded detection complex binds to a receptor on and/or in a cell at a location in the biological sample;
b) contacting the biological sample with a probe or probe set that hybridizes to the reporter oligonucleotide; and
c) detecting a signal associated with the probe or probe set or a product thereof at the location in the biological sample.

2. The method of claim 1, wherein the detection complex is bound to the antigen to form the antigen-loaded detection complex prior to the contacting in a).

3-4. (canceled)

5. The method of claim 1, wherein the antigen and the APM form an antigen-APM monomer, and the antigen-loaded detection complex comprises one or more dimers, tetramers, pentamers, octamers, streptamers, or dodecamers of the antigen-APM monomer.

6. The method of claim 1, wherein the antigen comprises a peptide or a protein.

7-8. (canceled)

9. The method of claim 1, wherein the APM is:
a Class I MHC molecule comprising an α subunit and a β2 microglobulin subunit, or
is a Class II MHC molecule comprising an α subunit and a β subunit.

10. (canceled)

11. The method of claim 1, wherein the antigen-loaded detection complex comprises a scaffold covalently or non-covalently conjugated to the APM monomer.

12. The method of claim 11, wherein the scaffold comprises a fluorochrome, a streptavidin, or an avidin.

13-14. (canceled)

15. The method of claim 12, wherein the antigen-loaded detection complex comprises four MHC molecules each linked to an affinity tag that binds to the scaffold.

16. The method of claim 15, wherein each of the four MHC molecules is bound to one molecule of an antigen peptide.

17. The method of claim 16, wherein the antigen peptide is between 5 and 40 amino acid residues in length, inclusive.

18-20. (canceled)

21. The method of claim 1, wherein the cell is a T cell, a B cell, or an NKT cell.

22. The method of claim 1, wherein the receptor is a T cell receptor (TCR).

23. The method of claim 1, wherein the reporter oligonucleotide is covalently or non-covalently conjugated to the antigen.

24. (canceled)

25. The method of claim 1, wherein the reporter oligonucleotide is covalently or non-covalently conjugated to a scaffold of the antigen-loaded detection complex.

26-27. (canceled)

28. The method of claim 1, wherein the reporter oligonucleotide comprises a barcode region for identifying the antigen or portion thereof, and wherein the barcode region comprises one or more barcode sequences.

29. The method of claim 1, wherein the biological sample is not fixed and/or crosslinked prior to the contacting in a).

30-37. (canceled)

38. The method of claim 1, further comprising fixing and/or crosslinking the biological sample after the contacting in a) and prior to the contacting in b).

39-43. (canceled)

44. The method of claim 1, wherein the antigen-loaded detection complex is not fluorescent or is not fluorescently labeled.

45-65. (canceled)

66. The method of claim 1, wherein the detecting in c) comprises sequential hybridization, sequencing by hybridization, sequencing by ligation, sequencing by synthesis, sequencing by binding, or a combination thereof, and/or
wherein the detecting in c) does not comprise contacting the biological sample with an antibody that binds to the antigen-loaded detection complex.

67-105. (canceled)

106. The method of claim 1, wherein the scaffold comprises dextran.
